SEQUENCE LISTING (59 sequences)

SEQ ID NO: 1 - DNA, 627 bp, E. coli
  misc_feature (1)..(627): tetR
atgatgtctagattagataaaagtaaagtgattaacagcgcattagagctgcttaatgaggtcggaatcgaaggtttaacaacccgtaaactcgcccagaagctaggtgtagagcagcctacattgtattggcatgtaaaaaataagcgggctttgctcgacgccttagccattgagatgttagataggcaccatactcacttttgccctttagaaggggaaagctggcaagattttttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattatatgcactcagcgctgtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtcttaa

SEQ ID NO: 2 - DNA, 103 bp, E. coli
  misc_feature (1)..(103): tet promoter
taattcctaattgctagcattgtacctaggactgagctagccataaagttgacactctatcgttgatagagttattttaccactccctatcagtgatagagaa

SEQ ID NO: 3 - DNA, 35 bp, Artificial Sequence
  Synthetic RBS for Cas9 #1
aagaattcaaaagatctaaagaggacttcggatct

SEQ ID NO: 4 - DNA, 4107 bp, Artificial Sequence
  Synthetic Cas9 #1
atggacaagaagtactctatagggcttgacatagggacgaatagcgtagggtgggctgtaataacggacgagtacaaggtaccctctaagaaattcaaggtacttgggaatacggaccgacactctataaagaagaatcttataggtgctcttcttttcgactctggggaaaccgctgaggctacgcgacttaagcggacggctcggcggcggtacacgcggcgaaagaatcgaatatgttatcttcaggagatattctctaatgagatggctaaggtagacgactctttcttccaccggcttgaagagtcattccttgtagaagaggataagaagcacgagcgacaccccatattcgggaatatagtagacgaggtagcttaccacgagaagtaccccacgatataccaccttcggaagaaacttgtagactctacggacaaggctgaccttcgacttatatatcttgctcttgctcacatgataaagttccgagggcacttccttatagagggggaccttaatcccgacaattctgacgtagacaagcttttcatacaacttgtacaaacgtacaatcaacttttcgaggaaaatcccataaatgcttctggggtagacgctaaggctatacttagcgctcggctttctaagtctcggcgacttgaaaaccttatagctcaacttcccggggagaagaagaacgggcttttcggtaatcttattgctctttctcttgggcttacgcccaatttcaagtctaatttcgaccttgctgaggatgctaaacttcaactttctaaggacacgtacgacgacgaccttgacaatcttcttgctcaaataggggaccaatacgctgacctttttcttgctgctaagaatctttcagacgctatacttctttctgacatacttcgggtaaatacggagataacgaaggctcccctttctgctagcatgataaagcggtacgacgagcaccaccaagaccttacgcttcttaaagcgctcgtacgacaacaacttccggagaagtacaaagagattttcttcgaccaatctaagaatgggtacgctgggtacattgacgggggtgcttctcaagaagagttctacaagttcataaagcccatacttgaaaagatggacgggacggaggaacttctcgtaaagcttaatcgggaggaccttcttcgaaagcaacgaacgttcgacaatgggtctataccccaccaaatacaccttggtgagcttcacgctattcttcgacgacaagaagatttttacccgttccttaaggacaatcgagaaaagatagagaagatacttacgttccggataccctactacgtagggccgcttgctcgaggtaattctcggttcgcttggatgacgcggaagtctgaggaaacgataacgccctggaatttcgaggaagtagtagacaagggggcgtcagctcaatctttcatagagcgaatgacgaatttcgataagaatcttcccaatgagaaggtacttcccaagcactctcttctttacgagtacttcacggtatataatgagcttacgaaagtaaaatacgtaacggagggtatgcggaagcccgctttcctttctggggagcaaaaaaaggctatagtagaccttcttttcaagacgaatcgaaaagtaacggtaaagcaacttaaagaggactacttcaagaaaatagagtgtttcgactcagtagaaatatcaggggtagaagatcgattcaatgcttcacttgggacctaccacgatcttcttaaaattataaaggacaaggacttccttgacaacgaggaaaatgaggacattcttgaagatatagtacttacgcttaccctttttgaggaccgggagatgatagaggaacgacttaaaacgtatgctcaccttttcgacgacaaagtaatgaagcaacttaagcgacgacggtacacggggtgggggcgactttctcgaaagcttataaatgggatacgagacaagcaatcagggaagaccatacttgatttccttaagtcagacgggttcgctaatcggaatttcatgcaacttatacacgacgactctcttacgtttaaagaggacatacaaaaagctcaagtatcagggcaaggggattctcttcacgagcacattgctaaccttgctgggtctcccgctattaagaaggggatacttcaaaccgtaaaggtagtagacgagctcgtaaaagtaatggggcgacacaagcccgagaatatagtaatagaaatggctcgggagaatcaaacgacgcaaaagggtcaaaagaattctcgggagcggatgaagcgaatagaagaggggataaaagagcttgggtctcaaatacttaaagaacaccccgtagaaaatacgcaacttcaaaatgagaagctttacctttactaccttcaaaacgggcgagatatgtacgtagaccaagaacttgacataaatcgactttcagactacgatgtagaccatatagtaccgcaatcttttcttaaggacgactcaatagacaataaggtacttacgcggtctgacaagaatcgagggaagtctgacaatgtaccctcagaagaggtagtaaagaagatgaagaattactggcgacaacttcttaatgctaagcttattacgcaacggaagttcgacaaccttacgaaggctgagcggggggggctttctgaacttgataaggctgggttcataaagcggcaacttgtagaaacgcgacaaataaccaagcacgtagcacaaatacttgactcacgaatgaataccaagtacgacgagaacgacaagcttatacgagaagtaaaagtaataacgcttaagtcaaagcttgtatcagatttccgaaaggatttccaattttacaaagtacgggagataaataattaccaccacgctcacgacgcttaccttaatgctgtagtaggtacggctcttataaaaaagtacccgaagcttgaatctgagttcgtatacggggactacaaggtatacgacgtacgaaagatgatagctaagtctgagcaagaaatagggaaggcgacggctaagtacttcttctactctaatataatgaattttttcaagacggagattacgcttgctaatggggagatacgaaagcgaccgcttatagagaccaatggggaaacgggggagatagtatgggataaggggcgagattttgctacggtacgaaaagtactttctatgccccaggtaaacatagtaaaaaagacggaggtacaaaccggggggttctctaaagagagcatacttcccaagcgaaattctgataagcttatagctcggaagaaggactgggacccgaagaagtacggggggttcgactctcccacggtagcttatagcgtacttgtagtagctaaagtagaaaaggggaagtcaaagaaacttaagagcgtaaaagagcttcttgggataacgataatggaacggtcttctttcgagaagaaccccatagactttcttgaagctaaggggtacaaagaagtaaaaaaggaccttataataaagcttccgaagtactcacttttcgagcttgaaaatgggcgaaagcggatgcttgctagcgctggggaacttcaaaagggtaatgaacttgctcttccctcaaaatatgtaaatttcctttaccttgcttctcactatgagaagcttaaggggtcacccgaggataacgagcaaaaacaactttttgtagaacaacacaagcactaccttgacgagataatagagcaaatatctgagttctcaaagcgggtaatacttgctgacgcgaaccttgacaaagtactttcagcttacaataagcaccgagataagcccatacgggagcaagctgagaacataatacacctttttacgcttacgaaccttggtgctccggctgctttcaagtactttgacacgacgatagaccgaaagcgatacacgtctacgaaagaggtacttgacgctacgcttatacaccaatctataacggggctttacgagacccgaatagaccttagccaacttggtggggattaa

SEQ ID NO: 5 - DNA, 500 bp, E. coli
  misc_feature (1)..(500): Upstream homology #1
tttctcttttggggcacggatttccgtgcccatttcacaagttggctgttatgcaggaatacacgaatcattccgataccacttttgcactgcgtaatatctcctttcgtgtgcccgggcgcacgcttttgcatccgctgtcgttaacctttcctgccgggaaagtgaccggtctgattggtcacaacggttctggtaaatccactctgctcaaaatgcttggccgtcatcagccgccgtcggaaggggagattcttcttgatgcccaaccgctggaaagctggagcagcaaagcgtttgcccgcaaagtggcttatttgccgcagcagcttcctccggcagaagggatgaccgtgcgtgaactggtggcgattggtcgttacccgtggcatggcgcgctggggcgctttggggcggcagatcgcgaaaaagtcgaggaagctatctcgctggttggcttaaaaccgctggcgcatcggctggtcgatagtctctctggc

SEQ ID NO: 6 - DNA, 500 bp, E. coli
  misc_feature (1)..(500): Downstream homology #1
caacgccgctgaatcttgttccgccagaagatattgcagatatgggcgtggactacgacggcaactttgtttgcagcggtggcatgcgtatcttgccggtctggaccagcgatccgcaatcgctgtgccagcagagcgagatgcagcagcagccgtcaggcaatccgtttgatcagtcttctcagccgcagcaacagccgcaacagcaacctgctcagcaagagcagaaagacagcgacggtgtagccggttggatcaaggatatgtttggtagtaattaacatctaagcgtgaaataccggatggcgagttgccatccggtaaaataacatcccatctaagatattaaccctttcttttcatctggttgtttattaacccttcaggaacgctcagattgcgtaccgcttgcgaacccgccagcgtttcgaatattatcttatctttataataatcattctcgtttacgttatcattcactttacatcagagatatacca

SEQ ID NO: 7 - DNA, 720 bp, Artificial Sequence
  Synthetic Gene insert 1
atgcgtaaaggcgaagaactgttcacgggcgtagttccgattctggtcgagctggacggcgatgtgaacggtcataagtttagcgttcgcggtgaaggtgagggcgacgcgaccaacggcaaactgaccctgaagttcatctgcaccaccggtaaactgccggtgccttggccgaccttggtgacgacgttgacgtatggcgtgcagtgttttgcgcgttatccggaccacatgaaacaacacgatttcttcaaatctgcgatgccggagggttacgtccaggagcgtaccatttccttcaaggatgatggctactacaaaactcgcgcagaggttaagtttgaaggtgacacgctggtcaatcgtatcgaattgaagggtatcgactttaaagaggatggtaacattctgggccataaactggagtataacttcaacagccataatgtttacattacggcagacaagcaaaagaacggcatcaaggccaatttcaagattcgccacaatgttgaggacggtagcgtccaactggccgaccattaccagcagaacaccccaattggtgacggtccggttttgctgccggataatcactatctgagcacccaaagcgtgctgagcaaagatccgaacgaaaaacgtgatcacatggtcctgctggaatttgtgaccgctgcgggcatcacccacggtatggacgagctgtataagtaatga

SEQ ID NO: 8 - DNA, 795 bp, E. coli
  misc_feature (1)..(795): Gene insert 2
atgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctga

SEQ ID NO: 9 - DNA, 102 bp, Artificial Sequence
  Synthetic tonA sgRNA #1
agacatgccgctaaccgctggttttagagctagaaatagcaagttaaaataaggctagtccgttatcaacttgaaaaagtggcaccgagtcggtgctttttt

SEQ ID NO: 10 - DNA, 546 bp, Artificial Sequence
  Synthetic p15A
tttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaa

SEQ ID NO: 11 - DNA, 265 bp, Artificial Sequence
  Synthetic AcrIIA4
atgaacatcaacgatttaatccgtgagattaagaacaaagattacacagtcaagttatcagggacagactccaactccatcactcaattaattatccgtgtgaacaatgatggtaacgaatatgttattagcgaatcggaaaatgagagtattgtcgagaagttcattagtgcgtttaagaatggctggaaccaagaatacgaagatgaagaagaattttataacgacatgcaaactatcactctgaagagcgagttgaactaaa

SEQ ID NO: 12 - DNA, 12 bp, Artificial Sequence
  Synthetic RBS for AcrIIA4
ttttgctactag

SEQ ID NO: 13 - DNA, 16 bp, Artificial Sequence
  Synthetic promoter for AcrIIA4
taggtactatgctagc

SEQ ID NO: 14 - DNA, 763 bp, E. coli
  misc_feature (1)..(763): Chloram-phenicol resistance
tgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaatttcgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaa

SEQ ID NO: 15 - DNA, 20 bp, Artificial Sequence
  Synthetic Primer 1
ttcgtcgagcaacagacaac

SEQ ID NO: 16 - DNA, 24 bp, Artificial Sequence
  Synthetic Primer 2
gacgctaccgtcctcaacattgtg

SEQ ID NO: 17 - DNA, 23 bp, Artificial Sequence
  Synthetic Primer 3
aaccagccgaccaaactgtatgg

SEQ ID NO: 18 - DNA, 28 bp, Artificial Sequence
  Synthetic Primer 4
atgattgaacaagatggattgcacgcag

SEQ ID NO: 19 - DNA, 4107 bp, Artificial Sequence
  Synthetic Cas9 #2
atggacaaaaaatattctatcggcttagatatcggtaccaattctgttggttgggcagtcattactgacgagtataaagttccgagcaaaaagttcaaagtcctgggtaataccgatcgccactctattaaaaagaatctgatcggcgcgttgctgtttgattcgggtgaaaccgcagaggcaacccgtctgaaacgtacggctcgccgtcgttatacccgtcgtaaaaaccgcatttgctacttgcaagaaatcttcagcaacgaaatggcgaaagtcgatgatagcttctttcaccgcctggaagagagcttcctggttgaggaagataagaaacacgaacgtcacccgatcttcggtaacatcgttgacgaagtggcataccatgaaaagtacccaacgatttatcatctgcgtaagaaactggttgacagcaccgataaagctgatctgcgcctgatctacctggcgctggcgcacatgattaaatttcgcggccatttcctgattgagggtgacctgaatccggataactccgatgttgacaagctgtttatccaattggttcaaacctataatcagctgtttgaagagaacccgatcaatgccagcggcgtcgacgcgaaagcgattcttagcgcccgtctgtctaagagccgtcgtttagaaaatctgattgcgcaactgccgggcgagaagaaaaacggcctgtttggtaatttgattgccctgagcctgggtctgaccccaaattttaaaagcaatttcgacttggcagaggatgctaagctgcaactgtccaaagacacgtacgatgacgatctggacaatttgttggcacagattggcgatcagtacgctgacctgttcctggcggcaaaaaacttgagcgatgcgattctgttgagcgacatcctgcgcgttaacacggagatcaccaaagccccgctgtctgctagcatgatcaagcgttatgacgagcaccaccaggatctgaccctgctgaaggcattggtccgtcaacagctgccggagaaatacaaagaaattttcttcgaccaatccaaaaatggttacgcgggctatattgacggtggcgcgagccaagaggaattttataagttcattaaaccgattttggagaaaatggacggtaccgaagaactgctggttaaactgaaccgcgaggatctgctgcgcaagcaacgcacgttcgataacggctctatcccgcaccagattcacctgggcgagctgcacgctatcctgcgtcgccaagaagatttctatccgttcctgaaagacaaccgtgaaaagatcgagaaaattctgacgttccgcattccgtactacgtgggtccattggcacgtggcaacagccgcttcgcttggatgacccgcaaatccgaggaaaccatcacgccttggaactttgaggaagttgtggataagggtgcgtccgcacagagcttcatcgagcgtatgaccaacttcgataagaatctgccgaatgaaaaagtgctgccgaaacatagcctgctgtatgagtatttcaccgtttataatgaactgaccaaagttaaatacgttaccgagggtatgcgtaagccagcgtttctgagcggcgagcaaaaaaaagcaattgtggatctgttgttcaaaaccaaccgcaaggtaaccgtgaaacagctgaaagaagattacttcaaaaagattgaatgtttcgacagcgttgaaatctcgggcgttgaggaccgttttaacgcttccctgggtacctatcatgatttgctgaaaatcatcaaagataaggacttcttggataacgaagaaaacgaagatattctggaagatattgtcttgacgctgacgctgtttgaagatcgcgagatgattgaggagcgtttgaaaacctacgcgcatttgtttgacgataaagtgatgaagcaactgaaacgccgtcgttacaccggttggggtcgcttatcgcgcaagttgattaacggtattcgcgacaaacagagcggcaaaactattttggattttctgaaatcggacggctttgcgaaccgtaatttcatgcagctgattcatgatgatagcttgaccttcaaagaggacattcaaaaagcccaggtcagcggtcagggcgacagcctccacgagcatattgcgaacctggctggttctccggcgatcaagaaaggcatcctgcagaccgtgaaagttgttgatgaactggtcaaagttatgggccgtcacaagccggaaaacattgtgattgagatggcgcgcgagaaccagaccacccaaaagggtcagaagaatagccgtgagagaatgaaacgtatcgaagaaggtattaaagaattgggcagccagattctgaaagagcatccggtcgaaaatacccagctgcaaaacgaaaaactgtacctgtactacttgcaaaatggtcgtgatatgtacgtggatcaggaactggacatcaaccgcctgagcgactatgatgttgatcacatcgtgccgcaatcctttctgaaggatgacagcatcgacaataaagttttgactcgctcggataagaatcgtggtaagtccgacaacgtgccgagcgaagaagtcgtgaagaaaatgaagaattattggcgtcaactgcttaatgccaaactgatcacccaacgtaagtttgacaatctgacgaaagccgagcgcggtggtctgagcgagctggataaggccggttttatcaagcgtcagctcgtcgaaacgcgtcagatcaccaaacatgtcgcacaaatcttagattcccgcatgaatacgaaatacgacgagaacgacaagctgatccgtgaagttaaagtgattaccctgaaatctaaactggtgagcgatttccgtaaagacttccagttttacaaggttcgcgagatcaacaattatcaccatgcccacgacgcttacctgaatgcagtggttggtaccgcactgatcaagaaatatccgaagctggagagcgagtttgtgtacggtgattacaaagtctacgatgtccgtaagatgatcgcaaaatctgaacaagagatcggtaaagcgacggcgaagtactttttctatagcaacattatgaacttttttaaaaccgaaatcaccctggccaacggcgagatccgcaagcgtccgctgatcgaaacgaacggcgaaacgggcgagattgtgtgggacaagggtcgcgacttcgctactgtccgtaaagtgctgagcatgcctcaggtgaatatcgtcaaaaagaccgaagttcagaccggtggtttcagcaaagagagcatcttgccgaagcgtaacagcgacaaactgattgcccgtaaaaaagattgggacccgaagaaatacggcggcttcgattcgccgaccgttgcatattcagttctggtcgtggcaaaagttgaaaagggcaagtccaaaaagttgaagtccgttaaagagctgctgggtattactattatggaacgcagctccttcgagaagaatccgattgacttcctggaagcgaagggctataaagaggttaagaaagatctgatcatcaagctgccgaagtacagcctgtttgagctggaaaatggccgtaaacgtatgctggcgtctgcaggcgaactgcaaaagggtaacgaactggcgctgccgagcaaatatgtcaattttctctatctggccagccactacgagaaactgaagggttctcctgaagataacgaacagaaacagctgtttgtcgagcagcataaacactatctggacgaaatcatcgaacagatcagcgagttctctaagcgtgtcatcctggctgacgcgaatctggacaaagtgctgtccgcatataacaagcaccgtgacaagccgatccgtgaacaggctgaaaacatcatccacctgtttaccctgacgaacttgggtgccccggcggcgttcaaatactttgacacgaccatcgatcgtaaacgttacacgagcactaaagaggtcctggacgcgacgctgattcaccaaagcatcacgggcctgtacgaaactcgtatcgacctgtcccaactgggtggcgactaa

SEQ ID NO: 20 - DNA, 4107 bp, Artificial Sequence
  Synthetic nCas9
atggacaaaaaatattctatcggcttagccatcggtaccaattctgttggttgggcagtcattactgacgagtataaagttccgagcaaaaagttcaaagtcctgggtaataccgatcgccactctattaaaaagaatctgatcggcgcgttgctgtttgattcgggtgaaaccgcagaggcaacccgtctgaaacgtacggctcgccgtcgttatacccgtcgtaaaaaccgcatttgctacttgcaagaaatcttcagcaacgaaatggcgaaagtcgatgatagcttctttcaccgcctggaagagagcttcctggttgaggaagataagaaacacgaacgtcacccgatcttcggtaacatcgttgacgaagtggcataccatgaaaagtacccaacgatttatcatctgcgtaagaaactggttgacagcaccgataaagctgatctgcgcctgatctacctggcgctggcgcacatgattaaatttcgcggccatttcctgattgagggtgacctgaatccggataactccgatgttgacaagctgtttatccaattggttcaaacctataatcagctgtttgaagagaacccgatcaatgccagcggcgtcgacgcgaaagcgattcttagcgcccgtctgtctaagagccgtcgtttagaaaatctgattgcgcaactgccgggcgagaagaaaaacggcctgtttggtaatttgattgccctgagcctgggtctgaccccaaattttaaaagcaatttcgacttggcagaggatgctaagctgcaactgtccaaagacacgtacgatgacgatctggacaatttgttggcacagattggcgatcagtacgctgacctgttcctggcggcaaaaaacttgagcgatgcgattctgttgagcgacatcctgcgcgttaacacggagatcaccaaagccccgctgtctgctagcatgatcaagcgttatgacgagcaccaccaggatctgaccctgctgaaggcattggtccgtcaacagctgccggagaaatacaaagaaattttcttcgaccaatccaaaaatggttacgcgggctatattgacggtggcgcgagccaagaggaattttataagttcattaaaccgattttggagaaaatggacggtaccgaagaactgctggttaaactgaaccgcgaggatctgctgcgcaagcaacgcacgttcgataacggctctatcccgcaccagattcacctgggcgagctgcacgctatcctgcgtcgccaagaagatttctatccgttcctgaaagacaaccgtgaaaagatcgagaaaattctgacgttccgcattccgtactacgtgggtccattggcacgtggcaacagccgcttcgcttggatgacccgcaaatccgaggaaaccatcacgccttggaactttgaggaagttgtggataagggtgcgtccgcacagagcttcatcgagcgtatgaccaacttcgataagaatctgccgaatgaaaaagtgctgccgaaacatagcctgctgtatgagtatttcaccgtttataatgaactgaccaaagttaaatacgttaccgagggtatgcgtaagccagcgtttctgagcggcgagcaaaaaaaagcaattgtggatctgttgttcaaaaccaaccgcaaggtaaccgtgaaacagctgaaagaagattacttcaaaaagattgaatgtttcgacagcgttgaaatctcgggcgttgaggaccgttttaacgcttccctgggtacctatcatgatttgctgaaaatcatcaaagataaggacttcttggataacgaagaaaacgaagatattctggaagatattgtcttgacgctgacgctgtttgaagatcgcgagatgattgaggagcgtttgaaaacctacgcgcatttgtttgacgataaagtgatgaagcaactgaaacgccgtcgttacaccggttggggtcgcttatcgcgcaagttgattaacggtattcgcgacaaacagagcggcaaaactattttggattttctgaaatcggacggctttgcgaaccgtaatttcatgcagctgattcatgatgatagcttgaccttcaaagaggacattcaaaaagcccaggtcagcggtcagggcgacagcctccacgagcatattgcgaacctggctggttctccggcgatcaagaaaggcatcctgcagaccgtgaaagttgttgatgaactggtcaaagttatgggccgtcacaagccggaaaacattgtgattgagatggcgcgcgagaaccagaccacccaaaagggtcagaagaatagccgtgagagaatgaaacgtatcgaagaaggtattaaagaattgggcagccagattctgaaagagcatccggtcgaaaatacccagctgcaaaacgaaaaactgtacctgtactacttgcaaaatggtcgtgatatgtacgtggatcaggaactggacatcaaccgcctgagcgactatgatgttgatcacatcgtgccgcaatcctttctgaaggatgacagcatcgacaataaagttttgactcgctcggataagaatcgtggtaagtccgacaacgtgccgagcgaagaagtcgtgaagaaaatgaagaattattggcgtcaactgcttaatgccaaactgatcacccaacgtaagtttgacaatctgacgaaagccgagcgcggtggtctgagcgagctggataaggccggttttatcaagcgtcagctcgtcgaaacgcgtcagatcaccaaacatgtcgcacaaatcttagattcccgcatgaatacgaaatacgacgagaacgacaagctgatccgtgaagttaaagtgattaccctgaaatctaaactggtgagcgatttccgtaaagacttccagttttacaaggttcgcgagatcaacaattatcaccatgcccacgacgcttacctgaatgcagtggttggtaccgcactgatcaagaaatatccgaagctggagagcgagtttgtgtacggtgattacaaagtctacgatgtccgtaagatgatcgcaaaatctgaacaagagatcggtaaagcgacggcgaagtactttttctatagcaacattatgaacttttttaaaaccgaaatcaccctggccaacggcgagatccgcaagcgtccgctgatcgaaacgaacggcgaaacgggcgagattgtgtgggacaagggtcgcgacttcgctactgtccgtaaagtgctgagcatgcctcaggtgaatatcgtcaaaaagaccgaagttcagaccggtggtttcagcaaagagagcatcttgccgaagcgtaacagcgacaaactgattgcccgtaaaaaagattgggacccgaagaaatacggcggcttcgattcgccgaccgttgcatattcagttctggtcgtggcaaaagttgaaaagggcaagtccaaaaagttgaagtccgttaaagagctgctgggtattactattatggaacgcagctccttcgagaagaatccgattgacttcctggaagcgaagggctataaagaggttaagaaagatctgatcatcaagctgccgaagtacagcctgtttgagctggaaaatggccgtaaacgtatgctggcgtctgcaggcgaactgcaaaagggtaacgaactggcgctgccgagcaaatatgtcaattttctctatctggccagccactacgagaaactgaagggttctcctgaagataacgaacagaaacagctgtttgtcgagcagcataaacactatctggacgaaatcatcgaacagatcagcgagttctctaagcgtgtcatcctggctgacgcgaatctggacaaagtgctgtccgcatataacaagcaccgtgacaagccgatccgtgaacaggctgaaaacatcatccacctgtttaccctgacgaacttgggtgccccggcggcgttcaaatactttgacacgaccatcgatcgtaaacgttacacgagcactaaagaggtcctggacgcgacgctgattcaccaaagcatcacgggcctgtacgaaactcgtatcgacctgtcccaactgggtggcgactaa

SEQ ID NO: 21 - DNA, 398 bp, Artificial Sequence
  Synthetic tonA sgRNA pair #1
aggtagattatcagctttcgctaaggatgatttctggaattcttccctatcagtgatagagattgacatccctatcagtgatagagatactgagcaccgcagttgtagtagccacaggttttagagctagaaatagcaagttaaaataaggctagtccgttatcaacttgaaaaagtggcaccgagtcggtgcttttttccacagattatcagctttcgctaaggatgatttctggaattcttccctatcagtgatagagattgacatccctatcagtgatagagatactgagcacttcttgcggcgcaggtgcaggttttagagctagaaatagcaagttaaaataaggctagtccgttatcaacttgaaaaagtggcaccgagtcggtgctttttt

SEQ ID NO: 22 - DNA, 398 bp, Artificial Sequence
  Synthetic tonA sgRNA pair #2
aggtagattatcagctttcgctaaggatgatttctggaattcttccctatcagtgatagagattgacatccctatcagtgatagagatactgagcaccgttatgatctggcgcgagtgttttagagctagaaatagcaagttaaaataaggctagtccgttatcaacttgaaaaagtggcaccgagtcggtgcttttttccacagattatcagctttcgctaaggatgatttctggaattcttccctatcagtgatagagattgacatccctatcagtgatagagatactgagcacgccataagtgttaaagcagcgttttagagctagaaatagcaagttaaaataaggctagtccgttatcaacttgaaaaagtggcaccgagtcggtgctttttt

SEQ ID NO: 23 - DNA, 398 bp, Artificial Sequence
  Synthetic tonA sgRNA pair #3
aggtagattatcagctttcgctaaggatgatttctggaattcttccctatcagtgatagagattgacatccctatcagtgatagagatactgagcacagacatgccgctaaccgctggttttagagctagaaatagcaagttaaaataaggctagtccgttatcaacttgaaaaagtggcaccgagtcggtgcttttttccacagattatcagctttcgctaaggatgatttctggaattcttccctatcagtgatagagattgacatccctatcagtgatagagatactgagcaccgttatgatctggcgcgagtgttttagagctagaaatagcaagttaaaataaggctagtccgttatcaacttgaaaaagtggcaccgagtcggtgctttttt

SEQ ID NO: 24 - DNA, 22 bp, Artificial Sequence
  Synthetic Insert DNA probe
catggtcctgctggaatttgtg

SEQ ID NO: 25 - DNA, 21 bp, Artificial Sequence
  Synthetic Target DNA probe
cgggacgaccgataaacgtga

SEQ ID NO: 26 - DNA, 4107 bp, Artificial Sequence
  Synthetic dCas9
atggacaaaaaatattctatcggcttagccatcggtaccaattctgttggttgggcagtcattactgacgagtataaagttccgagcaaaaagttcaaagtcctgggtaataccgatcgccactctattaaaaagaatctgatcggcgcgttgctgtttgattcgggtgaaaccgcagaggcaacccgtctgaaacgtacggctcgccgtcgttatacccgtcgtaaaaaccgcatttgctacttgcaagaaatcttcagcaacgaaatggcgaaagtcgatgatagcttctttcaccgcctggaagagagcttcctggttgaggaagataagaaacacgaacgtcacccgatcttcggtaacatcgttgacgaagtggcataccatgaaaagtacccaacgatttatcatctgcgtaagaaactggttgacagcaccgataaagctgatctgcgcctgatctacctggcgctggcgcacatgattaaatttcgcggccatttcctgattgagggtgacctgaatccggataactccgatgttgacaagctgtttatccaattggttcaaacctataatcagctgtttgaagagaacccgatcaatgccagcggcgtcgacgcgaaagcgattcttagcgcccgtctgtctaagagccgtcgtttagaaaatctgattgcgcaactgccgggcgagaagaaaaacggcctgtttggtaatttgattgccctgagcctgggtctgaccccaaattttaaaagcaatttcgacttggcagaggatgctaagctgcaactgtccaaagacacgtacgatgacgatctggacaatttgttggcacagattggcgatcagtacgctgacctgttcctggcggcaaaaaacttgagcgatgcgattctgttgagcgacatcctgcgcgttaacacggagatcaccaaagccccgctgtctgctagcatgatcaagcgttatgacgagcaccaccaggatctgaccctgctgaaggcattggtccgtcaacagctgccggagaaatacaaagaaattttcttcgaccaatccaaaaatggttacgcgggctatattgacggtggcgcgagccaagaggaattttataagttcattaaaccgattttggagaaaatggacggtaccgaagaactgctggttaaactgaaccgcgaggatctgctgcgcaagcaacgcacgttcgataacggctctatcccgcaccagattcacctgggcgagctgcacgctatcctgcgtcgccaagaagatttctatccgttcctgaaagacaaccgtgaaaagatcgagaaaattctgacgttccgcattccgtactacgtgggtccattggcacgtggcaacagccgcttcgcttggatgacccgcaaatccgaggaaaccatcacgccttggaactttgaggaagttgtggataagggtgcgtccgcacagagcttcatcgagcgtatgaccaacttcgataagaatctgccgaatgaaaaagtgctgccgaaacatagcctgctgtatgagtatttcaccgtttataatgaactgaccaaagttaaatacgttaccgagggtatgcgtaagccagcgtttctgagcggcgagcaaaaaaaagcaattgtggatctgttgttcaaaaccaaccgcaaggtaaccgtgaaacagctgaaagaagattacttcaaaaagattgaatgtttcgacagcgttgaaatctcgggcgttgaggaccgttttaacgcttccctgggtacctatcatgatttgctgaaaatcatcaaagataaggacttcttggataacgaagaaaacgaagatattctggaagatattgtcttgacgctgacgctgtttgaagatcgcgagatgattgaggagcgtttgaaaacctacgcgcatttgtttgacgataaagtgatgaagcaactgaaacgccgtcgttacaccggttggggtcgcttatcgcgcaagttgattaacggtattcgcgacaaacagagcggcaaaactattttggattttctgaaatcggacggctttgcgaaccgtaatttcatgcagctgattcatgatgatagcttgaccttcaaagaggacattcaaaaagcccaggtcagcggtcagggcgacagcctccacgagcatattgcgaacctggctggttctccggcgatcaagaaaggcatcctgcagaccgtgaaagttgttgatgaactggtcaaagttatgggccgtcacaagccggaaaacattgtgattgagatggcgcgcgagaaccagaccacccaaaagggtcagaagaatagccgtgagagaatgaaacgtatcgaagaaggtattaaagaattgggcagccagattctgaaagagcatccggtcgaaaatacccagctgcaaaacgaaaaactgtacctgtactacttgcaaaatggtcgtgatatgtacgtggatcaggaactggacatcaaccgcctgagcgactatgatgttgatgccatcgtgccgcaatcctttctgaaggatgacagcatcgacaataaagttttgactcgctcggataagaatcgtggtaagtccgacaacgtgccgagcgaagaagtcgtgaagaaaatgaagaattattggcgtcaactgcttaatgccaaactgatcacccaacgtaagtttgacaatctgacgaaagccgagcgcggtggtctgagcgagctggataaggccggttttatcaagcgtcagctcgtcgaaacgcgtcagatcaccaaacatgtcgcacaaatcttagattcccgcatgaatacgaaatacgacgagaacgacaagctgatccgtgaagttaaagtgattaccctgaaatctaaactggtgagcgatttccgtaaagacttccagttttacaaggttcgcgagatcaacaattatcaccatgcccacgacgcttacctgaatgcagtggttggtaccgcactgatcaagaaatatccgaagctggagagcgagtttgtgtacggtgattacaaagtctacgatgtccgtaagatgatcgcaaaatctgaacaagagatcggtaaagcgacggcgaagtactttttctatagcaacattatgaacttttttaaaaccgaaatcaccctggccaacggcgagatccgcaagcgtccgctgatcgaaacgaacggcgaaacgggcgagattgtgtgggacaagggtcgcgacttcgctactgtccgtaaagtgctgagcatgcctcaggtgaatatcgtcaaaaagaccgaagttcagaccggtggtttcagcaaagagagcatcttgccgaagcgtaacagcgacaaactgattgcccgtaaaaaagattgggacccgaagaaatacggcggcttcgattcgccgaccgttgcatattcagttctggtcgtggcaaaagttgaaaagggcaagtccaaaaagttgaagtccgttaaagagctgctgggtattactattatggaacgcagctccttcgagaagaatccgattgacttcctggaagcgaagggctataaagaggttaagaaagatctgatcatcaagctgccgaagtacagcctgtttgagctggaaaatggccgtaaacgtatgctggcgtctgcaggcgaactgcaaaagggtaacgaactggcgctgccgagcaaatatgtcaattttctctatctggccagccactacgagaaactgaagggttctcctgaagataacgaacagaaacagctgtttgtcgagcagcataaacactatctggacgaaatcatcgaacagatcagcgagttctctaagcgtgtcatcctggctgacgcgaatctggacaaagtgctgtccgcatataacaagcaccgtgacaagccgatccgtgaacaggctgaaaacatcatccacctgtttaccctgacgaacttgggtgccccggcggcgttcaaatactttgacacgaccatcgatcgtaaacgttacacgagcactaaagaggtcctggacgcgacgctgattcaccaaagcatcacgggcctgtacgaaactcgtatcgacctgtcccaactgggtggcgactaa

SEQ ID NO: 27 - DNA, 796 bp, Artificial Sequence
  Synthetic gfp, lacZ, gusA, flhC sgRNA
aggtagattatcagctttcgctaaggatgatttctggaattcttccctatcagtgatagagattgacatccctatcagtgatagagatactgagcacaaaggcgaagaactgttcacgttttagagctagaaatagcaagttaaaataaggctagtccgttatcaacttgaaaaagtggcaccgagtcggtgcttttttccacagattatcagctttcgctaaggatgatttctggaattcttccctatcagtgatagagattgacatccctatcagtgatagagatactgagcacagcggataacaatttcacacgttttagagctagaaatagcaagttaaaataaggctagtccgttatcaacttgaaaaagtggcaccgagtcggtgcttttttggtaagattatcagctttcgctaaggatgatttctggaattcttccctatcagtgatagagattgacatccctatcagtgatagagatactgagcaccggcctgtgggcattcagtcgttttagagctagaaatagcaagttaaaataaggctagtccgttatcaacttgaaaaagtggcaccgagtcggtgcttttttccgaagattatcagctttcgctaaggatgatttctggaattcttccctatcagtgatagagattgacatccctatcagtgatagagatactgagcacgcatctgcaaacgagcgcccgttttagagctagaaatagcaagttaaaataaggctagtccgttatcaacttgaaaaagtggcaccgagtcggtgctttttt

SEQ ID NO: 28 - DNA, 624 bp, Artificial Sequence
  Synthetic tetR #2
atgtcgcgcctggacaaatccaaggtgatcaacagcgcactggaactgctgaacgaagtgggcatcgaaggactgaccacgcgcaaactggcccagaagctgggcgtggaacagccgacactgtactggcatgtgaaaaacaagcgcgcactgctggacgccctggcaatcgaaatgctggaccgccatcacacccacttctgcccgctggaaggagaatcctggcaggacttcctgcgcaacaacgcaaaatcgttccgctgcgccctgctgtcccatagagacggcgcaaaagtgcacctgggaacgcgcccgacagaaaagcagtacgaaacgctggaaaaccagctggccttcctgtgccagcagggcttcagcctggaaaacgccctgtatgcactgtcggccgtgggccatttcaccctgggatgcgtgctggaagaccaggaacaccaggtggcaaaggaagaacgcgaaacgccgacgacggacagcatgccgccgctgctgagacaggcaatcgaactgttcgaccatcagggcgcagaaccggccttcctgttcggcctggaactgatcatctgcggactggaaaaacagctgaagtgcgaatccggaagctaa

SEQ ID NO: 29 - DNA, 43 bp, Artificial Sequence
  Synthetic RBS for tet
cgcattttaaaataaaataaattatttatgatattaaacgaat

SEQ ID NO: 30 - DNA, 138 bp, Bacteroides thetaiotaomicron
  misc_feature (1)..(138): P2 promoter for tet
aagaaaaggcgttttgtttttcttctttaccttctttccctttcgctaagagagtctgagaaacgatagaaaaagaaaagcgaaaaaacttccgaaaacatttggtagttaaaataaaacctcttacctttgcacccg

SEQ ID NO: 31 - DNA, 200 bp, Artificial Sequence
  Synthetic P1TDP-GH023  promoter for Cas9 #3
ttttgcacccgctttccaagagaagaaagccttgttaaattgacttagtgtaaaagcgcagtactgcttgaccataagaacaaaaaaatctctatcactgatagggataaagtttggaagataaagctaaaagttcttatctttgcagtctccctatcagtgatagagacgaaataaagacatataaaagaaaagacacc

SEQ ID NO: 32 - DNA, 4107 bp, Artificial Sequence
  Synthetic Cas9 #3
atggacaaaaagtattcgatcggcctggacatcggaacaaactccgtgggctgggccgtgatcaccgacgaatacaaggtgccgtcgaaaaagttcaaagtgctgggaaacacggaccgccattccatcaaaaagaacctgatcggcgccctgctgttcgacagcggagaaacggccgaagcaacgagactgaaacgcaccgcacgccgccgctacacacgtcgcaaaaaccgcatctgctacctgcaggaaatcttctccaacgaaatggcaaaagtggacgacagcttcttccaccgcctggaagaatcgttcctggtggaagaagacaaaaagcatgaacgccacccgatcttcggcaacatcgtggacgaagtggcctatcatgaaaagtacccgacgatctatcatctgcgcaaaaaactggtggactccacagacaaggcagacctgcgcctgatctatctggccctggcacacatgatcaaattccgcggccacttcctgatcgaaggagacctgaacccggacaacagcgacgtggacaaactgttcatccagctggtgcagacatacaaccagctgttcgaagaaaacccgatcaacgccagcggcgtggacgccaaggcaatcctgtccgcaagactgtcgaaatcccgccgcctggaaaacctgatcgcccagctgccgggcgaaaagaaaaacggcctgttcggaaacctgatcgcactgtccctgggactgaccccgaacttcaaaagcaacttcgacctggccgaagacgcaaagctgcagctgtccaaagacacgtatgacgacgacctggacaacctgctggcccagatcggagaccagtacgcagacctgttcctggccgcaaagaacctgagcgacgccatcctgctgtcggacatcctgcgcgtgaacaccgaaatcacgaaggccccgctgagcgcctccatgatcaaacgctatgacgaacatcaccaggacctgaccctgctgaaagcactggtgcgccagcagctgccggaaaaatacaaggaaatcttcttcgaccagtcgaagaacggctacgccggatatatcgacggcggagcatcccaggaagaattttacaaattcatcaagccgatcctggaaaaaatggacggcacagaagaactgctggtgaagctgaaccgcgaagacctgctgcgcaaacagcgcaccttcgacaacggcagcatcccgcatcagatccacctgggagaactgcatgccatcctgcgtcgccaggaagacttctacccgttcctgaaggacaaccgcgaaaaaatcgaaaagatcctgacattccgcatcccgtactatgtgggaccgctggcccgcggaaactcgcgcttcgcatggatgacccgcaagtccgaagaaacgatcacaccgtggaacttcgaagaagtggtggacaaaggagcctccgcacagagcttcatcgaacgcatgaccaacttcgacaagaacctgccgaacgaaaaggtgctgccgaaacactcgctgctgtacgaatatttcaccgtgtataacgaactgacgaaagtgaagtacgtgacagaaggcatgcgcaaaccggccttcctgtccggagaacagaaaaaggcaatcgtggacctgctgttcaagaccaaccgcaaagtgacggtgaaacagctgaaggaagactatttcaaaaagatcgaatgcttcgactccgtggaaatcagcggcgtggaagaccgcttcaacgcctccctgggaacgtaccatgacctgctgaaaatcatcaaagacaaggacttcctggacaacgaagaaaacgaagacatcctggaagacatcgtgctgaccctgacgctgttcgaagaccgcgaaatgatcgaagaacgcctgaagacgtatgcccacctgttcgacgacaaagtgatgaagcagctgaaacgccgccgctacacaggatggggacgcctgagccgcaaactgatcaacggcatccgcgacaagcagagcggaaaaacgatcctggacttcctgaagtcggacggcttcgcaaaccgcaacttcatgcagctgatccatgacgacagcctgacattcaaggaagacatccagaaagcacaggtgtcgggacagggagactccctgcatgaacacatcgccaacctggcaggctcgccggcaatcaaaaagggaatcctgcagaccgtgaaagtggtggacgaactggtgaaggtgatgggccgccacaaaccggaaaacatcgtgatcgaaatggcccgcgaaaaccagaccacgcagaagggacagaaaaactcccgcgaacgcatgaagcgcatcgaagaaggcatcaaagaactgggaagccagatcctgaaggaacatccggtggaaaacacgcagctgcagaacgaaaaactgtatctgtactatctgcagaacggccgcgacatgtacgtggaccaggaactggacatcaaccgcctgtcggactatgacgtggaccacatcgtgccgcagtccttcctgaaggacgacagcatcgacaacaaagtgctgacacgctccgacaagaaccgcggaaaatccgacaacgtgccgagcgaagaagtggtgaaaaagatgaaaaactactggcgccagctgctgaacgccaagctgatcacccagcgcaaattcgacaacctgacgaaggccgaacgcggcggactgagcgaactggacaaggcaggcttcatcaaacgccagctggtagaaacccgccagatcacgaagcatgtggcacagatcctggactcgcgcatgaacaccaaatacgacgaaaacgacaagctgatccgcgaagtgaaagtgatcacgctgaaatcgaagctggtgtccgacttccgcaaggacttccagttctataaagtgcgcgaaatcaacaactatcatcacgcccacgacgcatacctgaacgccgtggtgggcacagcactgatcaaaaagtacccgaagctggaaagcgaatttgtgtacggagactataaagtgtacgacgtgcgcaagatgatcgccaaaagcgaacaggaaatcggaaaggccaccgcaaagtatttcttctactcgaacatcatgaacttcttcaagacagaaatcaccctggccaacggcgaaatccgcaaacgcccgctgatcgaaacaaacggcgaaaccggagaaatcgtgtgggacaagggacgcgacttcgcaacggtgcgcaaagtgctgtccatgccgcaggtgaacatcgtgaaaaagacggaagtgcagacaggcggattctcgaaagaatccatcctgccgaagcgcaacagcgacaaactgatcgcccgcaaaaaggactgggacccgaaaaagtatggcggattcgactcgccgaccgtggcctactccgtgctggtagtggcaaaagtggaaaagggcaagtcgaaaaagctgaagtccgtgaaagaactgctgggaatcacgatcatggaacgctccagcttcgaaaagaacccgatcgacttcctggaagcaaagggctataaagaagtgaaaaaggacctgatcatcaagctgccgaaatacagcctgttcgaactggaaaacggacgcaaacgcatgctggcctcggcaggcgaactgcagaagggaaacgaactggccctgccgtccaaatacgtgaacttcctgtatctggcatcccattacgaaaaactgaagggaagcccggaagacaacgaacagaagcagctgttcgtggaacagcataaacactatctggacgaaatcatcgaacagatcagcgaattttcgaagcgcgtgatcctggccgacgcaaacctggacaaagtgctgtccgcctacaacaagcatcgcgacaaaccgatccgcgaacaggcagaaaacatcatccacctgttcaccctgacgaacctgggagcacctgcagcattcaaatatttcgacacaaccatcgaccgcaagcgctacacgagcacaaaagaagtgctggacgcaaccctgatccaccagtccatcacaggactgtacgaaacgagaatcgacctgagccagctgggcggagactaa

SEQ ID NO: 33 - DNA, 110 bp, Artificial Sequence
  Synthetic oriT
ccggccagcctcgcagagcaggattcccgttgagcaccgccaggtgcgaataagggacagtgaagaaggaacacccgctcgcgggtgggcctacttcacctatcctgccc

SEQ ID NO: 34 - DNA, 389 bp, Artificial Sequence
  Synthetic R6K origin
gatctgaagatcagcagttcaacctgttgatagtacgtactaagctctcatgtttcacgtactaagctctcatgtttaacgtactaagctctcatgtttaacgaactaaaccctcatggctaacgtactaagctctcatggctaacgtactaagctctcatgtttcacgtactaagctctcatgtttgaacaataaaattaatataaatcagcaacttaaatagcctctaaggttttaagttttataagaaaaaaaagaatatataaggcttttaaagcttttaaggtttaacggttgtggacaacaagccagggatgtaacgcactgagaagcccttagagcctctcaaagcaattttgagtgacacaggaacacttaacggctgaca

SEQ ID NO: 35 - DNA, 1536 bp, Bacteroides thetaiotaomicron
  misc_feature (1)..(1536): Upstream homology #2
tcctgctaatactatcacaatctcgccccggggctcggtagcggtaaagtgttctatcaattccgccaaacttccccgaacggtttcttcgtggagtttggaaatttcgcgagacacggttgcctgacgttcagtgccgaaatattcggcaaactgtgtcaacgttttcaacaagcggtgaggcgattcataaaacaccatagtacgatgttcttctgccaatgccttcagtcgtgtctgtcgtcctttcttctgagggaggaacccttcgaagcagaacttttcattcggcagtcccgatgctaccaatgccggaacaaaagctgtcgctcccggcaaacattgcacttcgataccgttgcgcacacattcgcggactaccaaaaatcccggatcagaaatcccgggtgttccggcatccgaaatcaatgccacggtttcacctgcctttattctattaacaacactttccaccgttttatgttcattaaatttgtgatgagattgcattgcattctttatttcaaagtgtttcagcaaaataccggaagtacgtgtatcctctgccaaaatcagatcgacctctttcagcaccctgattgcccgaaaggtcatgtcctctagatttcctaccggcgtaggtactacatataactttcccataaacttttatgctgattgattaaaatacttttttagcaactccaaaaagtcgatcatcgcctcattctcttcgtttacgctgactttcgaggcaagaaatgccacggcagtcttataagaactcatgactgatatctgctcgataacacggttcatcagttcggggtctccgccgaatatctcacgcgagaaacggaaagaatcattcaggctgatggaatgacgtaatccggcagccatctttatgctttcgcccaatacggcagttttcggctcctctatcagaagcgattcgtcgtcttccgactcatcttccgcttctttctcttccatttcgtcttcgacaacaggttcctcaattactgcttcttccacaacagtctgcggttcctgtactatcacaggttcatcttctcccggtgctgtcgcttcattctcttccacgaccttctcttctatcaccggacattcaacttcctctattacaggggcttgttcttcaacaatgggggcttcactttccgcttccgctacaggagaaggcgaggcttccaccggcacagcacttatctcttccgacaactgttccaaacgctcctgcatacgtaggatgctccgcttcaacagttcagacaaagtctgagtcggctctttagaaaacgtattcatgagtagcttcagctcatgaacatccagctcaatatctgttaatagcttctgtttcatttccatccattgttattatggtgcgaatgtaggaataatttatgcaataacttaaaataatggataatttaacagttcttgtacatcgaattgtttacctttgtccactcatttatttaatcaagaaagcacaaaaatcacatggta

SEQ ID NO: 36 - DNA, 1553 bp, Bacteroides thetaiotaomicron
  misc_feature (1)..(1553): Downstream homology #2
aggacggacagaagatataaactctatttaaaacatatatggaaagaaagaagataacattcgacagttttatacgtggttccatcggatgtgtactggtggtagggatactaatgcttgtggaacggctcagcggagtgttattacctttctttatagcctggctgatcgcctacatggtttatccgttagtcaagtttttccagtataagctacggttaaagagccgtattgtttctatcttctgctccttgtttctgatcactcttgtcggagtatccttattttatctgttggtacctcccatgatttcggaaataggcaggatgaatgacttactggtaacctacctcaccaatggagccgggaataatgtgcccaagaatctttccgaattcattcatgagaatattgatcttcaggcgctcaaccgtatattaagcgaagagaatattcttgcagccatcaaagatacagtgcccagagtttgggccctgcttgcggagtcgctcaatatcctgttcagcattctcgcctcctttatcatattactatatgtaatctttatattgctggattatgaagtcatagccgaaggatggctgcatctgctgcccaacaagtatcgtaccttcgcatccaacctcgtacatgatgtacaggacggcatgaatcggtatttccgtggtcaggcactggttgccttttgtgtggggattctgttcagtataggcttccttattatcgattttccgatggctatcgctctagggcttttcattggagcgcttaatatggttccttacctgcaaatcatcggtttcctccctacggtcctattggcgatccttaaagctgccgatacgggagagaatttctggattatcattgcatgcgcactggcagtcttcgccatcgtccaaattatacaggatactttcctcgtccccaaaatcatgggaaagattacggggctgaatcctgccatcatccttttatccctttctatatggggttcattaatgggaatgctgggcatgatcattgccttgcctctgactactttgatgctttcctactatcaacgctttattatcaacaaagaaagaataaaatatgatgaagtagaaactactgataatcaagaaacaagcgataaagaggaaaaataattggaacttttttctacgaaacacttgcaaattaaataaaagtaactacctttgcacccgcaatcaggaaataactgattcgcaaataaggattgattcagtagctcagcaggtagagcacaacacttttaatgttggggtcctgggttcgagccccagctggatcacttagaagaaatgaaaatactccgacaaactaagacaaagctctttaaaccagtggtttaaggagcttttttcttacccgcctacgacagaaaaaagacctcaaaaagccaccctgtgaagattaatcgttactaattcgttacggttttccgttacgataaaaaccgtaacgaatttataggtaagtcgctcatttgtcgaatattggcagtc

SEQ ID NO: 37 - DNA, 1709 bp, Bacteroides thetaiotaomicron
  misc_feature (1)..(1709): Bacteroides origin of replication
cgaaataagcgtcaatatcctcactgcttttgagggtaacacactttccggatttgatttcttcccttgccttgtcaatccttgcttgcagctccggggttatcatcaaatcttcacgaccaacttttaccaaagcgtaaatctcgtttttcctgcgaattagtacctgctcgccattgtcagctttggtgaaagacgctgcgaggttgttacggtattctcttactgataatgcttccatatctgtttgcttttaaattcagcacaaagatagctatatttcaataaaatacaaacattttgtacacaaacgtgtacacgccataaaaacccgtttccaatcctaccgcccgttggttggttttgctttgctctttttccctatcgtttttctttttccgacagtaaatcaaccacttagctctataaattccccttgtctgttataagcatcgtctgtaatatgctctatcacttcttcgcaaacgcccataaataaagggattagaacatgtgtttccccacactttgggagtttagtccccacactttgggagtttaaaagtaaattagtccccacactttgggagttttacccctctttttttgttttaatgtggggattgtgtatatttgcagcaaatcaatcgtttatgaaaaagaaactacctattaccaagaataaagatgttgttgtttcatgggtatatacatggtcaaaacagcaagacatgtccatacacgaacaaagaatagttcttcgcatactggaagcgtgccaagctgaactaaagggagtaaaactgaaagattatgcaggcacgaaacgcaagtttgagcatggactttgggatgttgatgcacaaatgcatgtatctgacgttattttttccggacgtgattacaatgaaatcatagccgcactcgattctttggcaggacgtttttttacttacgaagatgacgaggaatggtggaagtgcggatttatatctaacccaaaatataaaaaacgcactggcattataacctttcgtgtgtctaacgacctttgggatgtgtttaccaagttcgccaaggggtacagagaatttgagttaaacaaggctcttgcgctacctactgggtattcccttaggttttacatgttaatgagtgggcaggtgtaccctttggatatatcccttgaaaacttgaaagaccgtcttggcatacctgcggacaaatacaaggacaagaacggaaaagacagaatagatcattttgaggaaagagttttgaaaccagcaaaggctgcgcttgatgagagctgcccatacactttcaactacgtgaaagtgcgtgaaaacccaaacaacaaacgtagcaaggtgaccgggtttaggttctacccggtctatcaaccacagttcagagatgaagaactggaagggaaagaattgcaagcaaaggttacagcacgatatcagatagacagccatgtgtacgagtacctccgttattcctgcggcttcacatcggaagagataaaccggaacaaagagacattcataacggcacaagaaaagataaccgaccttatcggagaactggctctcctaaacggaaaatcacgagaaaagaacaatccgaaagggtggattataaacgcccttaaagggaaaatcaaggataagtaacaagattacccggctgaaacactccgggta

SEQ ID NO: 38 - DNA, 402 bp, Artificial Sequence
  Synthetic tdk sgRNA
aagatttgtatcattcggatttggtagacgatattctgagtattaagacttattatgaacaacagtggctcgatcgtggtttaaatatcaaatatatcaagttccgtcttccgcaagagggagttttgcaggaaccggatgtggaaattgaacttgatccgtaccgtagctacaaccgtagcaagcggagcggactgcaaacgagcaaatgataagtaataagtgacaagtaataagtaacaagtaataatagtaattgggtggaactatcaattttccactctcaattctgaactaattatactagccgatgaatcgatgttttagagctagaaatagcaagttaaaataaggctagtccgttatcaacttgaaaaagtggcaccgagtcggtgctttttt

SEQ ID NO: 39 - DNA, 735 bp, Artificial Sequence
  Synthetic ErmG resistance gene
atgaacaaagtaaatataaaagatagtcaaaattttattacttcaaaatatcacatagaaaaaataatgaattgcataagtttagatgaaaaagataacatctttgaaataggtgcagggaaaggtcattttactgctggattggtaaagagatgtaattttgtaacggcgatagaaattgattctaaattatgtgaggtaactcgtaataagctcttaaattatcctaactatcaaatagtaaatgatgatatactgaaatttacatttcctagccacaatccatataaaatatttggcagcataccttacaacataagcacaaatataattcgaaaaattgtttttgaaagttcagccacaataagttatttaatagtggaatatggttttgctaaaatgttattagatacaaacagatcactagcattgctgttaatggcagaggtagatatttctatattagcaaaaattcctaggtattatttccatccaaaacctaaagtggatagcacattaattgtattaaaaagaaagccagcaaaaatggcatttaaagagagaaaaaaatatgaaacttttgtaatgaaatgggttaacaaagagtacgaaaaactgtttacaaaaaatcaatttaataaagctttaaaacatgcgagaatatatgatataaacaatattagtttcgaacaatttgtatcgctatttaatagttataaaatatttaacggctaa

SEQ ID NO: 40 - DNA, 861 bp, Artificial Sequence
  Synthetic Carb resistance gene
atgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaa

SEQ ID NO: 41 - DNA, 372 bp, Artificial Sequence
  Synthetic AcrIIA2
atgacgctgacccgcgctcagaaaaaatacgccgaggcgatgcatgagtttatcaatatggttgatgactttgaagaatcaacgcctgactttgcaaaagaggttctgcacgactccgactatgtggtcattacaaaaaacgagaaatatgccgtggcactctgtagtctctccacagatgaatgtgagtacgatactaacttgtatttggatgaaaagctcgtcgattacagcacagttgatgtcaacggagtgacatattacatcaatatagtggaaacaaatgacatagatgatcttgaaattgcgaccgacgaggacgagatgaagtctggaaaccaagagattattcttaagtccgaactgaagtaa

SEQ ID NO: 42 - DNA, 25 bp, Artificial Sequence
  Synthetic Primer 5
gaagccaacatggctgcgcacataa

SEQ ID NO: 43 - DNA, 25 bp, Artificial Sequence
  Synthetic Primer 6
gatcaccgtccaccgtgatacgtac

SEQ ID NO: 44 - DNA, 4107 bp, Streptococcus pyogenes
  misc_feature (1)..(4107): Cas9
atggataagaaatactcaataggcttagatatcggcacaaatagcgtcggatgggcggtgatcactgatgaatataaggttccgtctaaaaagttcaaggttctgggaaatacagaccgccacagtatcaaaaaaaatcttataggggctcttttatttgacagtggagagacagcggaagcgactcgtctcaaacggacagctcgtagaaggtatacacgtcggaagaatcgtatttgttatctacaggagattttttcaaatgagatggcgaaagtagatgatagtttctttcatcgacttgaagagtcttttttggtggaagaagacaagaagcatgaacgtcatcctatttttggaaatatagtagatgaagttgcttatcatgagaaatatccaactatctatcatctgcgaaaaaaattggtagattctactgataaagcggatttgcgcttaatctatttggccttagcgcatatgattaagtttcgtggtcattttttgattgagggagatttaaatcctgataatagtgatgtggacaaactatttatccagttggtacaaacctacaatcaattatttgaagaaaaccctattaacgcaagtggagtagatgctaaagcgattctttctgcacgattgagtaaatcaagacgattagaaaatctcattgctcagctccccggtgagaagaaaaatggcttatttgggaatctcattgctttgtcattgggtttgacccctaattttaaatcaaattttgatttggcagaagatgctaaattacagctttcaaaagatacttacgatgatgatttagataatttattggcgcaaattggagatcaatatgctgatttgtttttggcagctaagaatttatcagatgctattttactttcagatatcctaagagtaaatactgaaataactaaggctcccctatcagcttcaatgattaaacgctacgatgaacatcatcaagacttgactcttttaaaagctttagttcgacaacaacttccagaaaagtataaagaaatcttttttgatcaatcaaaaaacggatatgcaggttatattgatgggggagctagccaagaagaattttataaatttatcaaaccaattttagaaaaaatggatggtactgaggaattattggtgaaactaaatcgtgaagatttgctgcgcaagcaacggacctttgacaacggctctattccccatcaaattcacttgggtgagctgcatgctattttgagaagacaagaagacttttatccatttttaaaagacaatcgtgagaagattgaaaaaatcttgacttttcgaattccttattatgttggtccattggcgcgtggcaatagtcgttttgcatggatgactcggaagtctgaagaaacaattaccccatggaattttgaagaagttgtcgataaaggtgcttcagctcaatcatttattgaacgcatgacaaactttgataaaaatcttccaaatgaaaaagtactaccaaaacatagtttgctttatgagtattttacggtttataacgaattgacaaaggtcaaatatgttactgaaggaatgcgaaaaccagcatttctttcaggtgaacagaagaaagccattgttgatttactcttcaaaacaaatcgaaaagtaaccgttaagcaattaaaagaagattatttcaaaaaaatagaatgttttgatagtgttgaaatttcaggagttgaagatagatttaatgcttcattaggtacctaccatgatttgctaaaaattattaaagataaagattttttggataatgaagaaaatgaagatatcttagaggatattgttttaacattgaccttatttgaagatagggagatgattgaggaaagacttaaaacatatgctcacctctttgatgataaggtgatgaaacagcttaaacgtcgccgttatactggttggggacgtttgtctcgaaaattgattaatggtattagggataagcaatctggcaaaacaatattagattttttgaaatcagatggttttgccaatcgcaattttatgcagctgatccatgatgatagtttgacatttaaagaagacattcaaaaagcacaagtgtctggacaaggcgatagtttacatgaacatattgcaaatttagctggtagccctgctattaaaaaaggtattttacagactgtaaaagttgttgatgaattggtcaaagtaatggggcggcataagccagaaaatatcgttattgaaatggcacgtgaaaatcagacaactcaaaagggccagaaaaattcgcgagagcgtatgaaacgaatcgaagaaggtatcaaagaattaggaagtcagattcttaaagagcatcctgttgaaaatactcaattgcaaaatgaaaagctctatctctattatctccaaaatggaagagacatgtatgtggaccaagaattagatattaatcgtttaagtgattatgatgtcgatcacattgttccacaaagtttccttaaagacgattcaatagacaataaggtcttaacgcgttctgataaaaatcgtggtaaatcggataacgttccaagtgaagaagtagtcaaaaagatgaaaaactattggagacaacttctaaacgccaagttaatcactcaacgtaagtttgataatttaacgaaagctgaacgtggaggtttgagtgaacttgataaagctggttttatcaaacgccaattggttgaaactcgccaaatcactaagcatgtggcacaaattttggatagtcgcatgaatactaaatacgatgaaaatgataaacttattcgagaggttaaagtgattaccttaaaatctaaattagtttctgacttccgaaaagatttccaattctataaagtacgtgagattaacaattaccatcatgcccatgatgcgtatctaaatgccgtcgttggaactgctttgattaagaaatatccaaaacttgaatcggagtttgtctatggtgattataaagtttatgatgttcgtaaaatgattgctaagtctgagcaagaaataggcaaagcaaccgcaaaatatttcttttactctaatatcatgaacttcttcaaaacagaaattacacttgcaaatggagagattcgcaaacgccctctaatcgaaactaatggggaaactggagaaattgtctgggataaagggcgagattttgccacagtgcgcaaagtattgtccatgccccaagtcaatattgtcaagaaaacagaagtacagacaggcggattctccaaggagtcaattttaccaaaaagaaattcggacaagcttattgctcgtaaaaaagactgggatccaaaaaaatatggtggttttgatagtccaacggtagcttattcagtcctagtggttgctaaggtggaaaaagggaaatcgaagaagttaaaatccgttaaagagttactagggatcacaattatggaaagaagttcctttgaaaaaaatccgattgactttttagaagctaaaggatataaggaagttaaaaaagacttaatcattaaactacctaaatatagtctttttgagttagaaaacggtcgtaaacggatgctggctagtgccggagaattacaaaaaggaaatgagctggctctgccaagcaaatatgtgaattttttatatttagctagtcattatgaaaagttgaagggtagtccagaagataacgaacaaaaacaattgtttgtggagcagcataagcattatttagatgagattattgagcaaatcagtgaattttctaagcgtgttattttagcagatgccaatttagataaagttcttagtgcatataacaaacatagagacaaaccaatacgtgaacaagcagaaaatattattcatttatttacgttgacgaatcttggagctcccgctgcttttaaatattttgatacaacaattgatcgtaaacgatatacgtctacaaaagaagttttagatgccactcttatccatcaatccatcactggtctttatgaaacacgcattgatttgagtcagctaggaggtgactga

SEQ ID NO: 45 - DNA, 1197 bp, Lactobacillus paracasei
  misc_feature (1)..(1197): LSEI_2368 (example protein target)
atgccagatttagcacagtcaacgtttattttgccgcaagatgcgacactgacctcagaacagtcagcattggaacagcgaatttgggcctttatcaacgacaatcacaacacaacgtcaacacatctattgatcatttcgggtgatgccggtgctggtaaaagtgtggtgttggatgctgcttttgcgcagttgcaaaaagctgcccgtgcgacaagcggcgagttggctggcacagacaacaagttgttggtaaatcacaatgaaatgctgaagatttacaaagaaattgctggtacaaagtcttattttcgaaagaaagactttatgaaaccaacaccatttattaacgcgtaccgcaaagccggtaagcgggcagatgtcgtgctgatcgatgaaggccatttactactgacgatgcctgatccgtataataaattccgtgggcataatcagcttgctgatattcttcagttggctcgagtggtggtgctcgtgttcgatttccaccagctagtcaagctaaagagtttttggacacctgctttattgaaaagagtgacgcgcgattacgcagtgacacattatcatctgaccgaacagatgcgggtaggcgatgcggcggtgaatgattggattgatcattttgttcgtggaaaaatgttgcctttgccacatccgcaacattttgattttcaagttttttctgatggtcagccgatgtatgacctcattcagcggcgcgatgccgaaaccggcctttcgcgcatggtcgcgacagctgactatccattcacagtgctcggtggtaaaacctggtatgtccaagctggctctctgcggttgccttgggacaagatcaacttcaccgatcgcccgtgggcacaacgtcctgagacattgcatgaagttggttccatttatactattcaaggctttgacttgaattacgcaggtgtgattctagggccgagtctaggctatgatccctcaaaagaccgacttacggttgatctggcgcagtaccaagacaaggaagcattcaaaaagcgcccggatttggccgacaccaccgacgctaaagccgcgattatcatgaatgccattaatattctgctgaagagggcgaagcatgggctttatctttacgcagctgatcctgctttgcgccaacggttattgcaattggccaaataa

SEQ ID NO: 46 - DNA, 997 bp, Lactobacillus paracasei
  misc_feature (1)..(997): Upstream homology arm 3.1
catacgaacacctccgttaacaacaatacagcattctggcgtgcaaacgctgctgtgacttgattttgtcggtacttttaaaacattttaaaaaagaacattaaatatacacaacaattaacagcatattacatactttaacccagtaaaccggttacactagagatagtcaatttctcgaaaaagttattcaagaactaggagtattgaaatgccttcaattgctgattaccctcaacgccatagcttggcgtcatttcaacaaacaccgctcactgaacttgatgcgggtttatttgcgcaactcggttatttgaacttcaattacctgatcggccagccctatgcgcgttttgccgatttaaatgacagtacccgcctaaacagggctaccttgacaacttgggcgattccaactcatcagatcatgttagacgccatgcgtcacagtgaacgttttgcccgggttacttgggaaaactggctggaaacttgcagccatcgcaacgaagaagacttcgcggccatcacattcacattagcccccggggtttattgtgtcagttttcgggggacaaccaataaactggttggttggaaagaagatctcaatatgagcttcatgccaacgattccagcgcagcgtcgggaattaagttacttgattaaacaaatcagtcagcatcccggcacttattacctgactggtcattccaaaggcggcagtatcgccacctatgcgtttgaccacttgccacaaccattagccagtcaagttgctcatgtctatagcttcgatgggccgagcggtgtcccgcttgatccaagccatcgtgaccgtgtcaccaagctcgttccacaaagctccttaattggcgtgagtctcgatccagcgatgaattttgaggttgtcaaaagtcgcgtgaagttgttcggccaacacgatgttcttacttggaacattgccgacacgaccttcgctcatttgccaaccaca

SEQ ID NO: 47 - DNA, 1003 bp, Lactobacillus paracasei
  misc_feature (1)..(1003): Downstream homology arm 3.2
acaacggcgctgccgacctgggtcagcatcaacatcgccaacatgacgatatggatcaacttcttaggcatctgtgcgcctccttccattgtttccggcgtctctcttcttccaacaccagcttaacaagttgaccccgcgcaaaggaatgataatgttcgttatttaacaagacatttttaacagtgcatacttattgacattataggtattaatctgtacatataatgtaaaaagctctatttataggtaaataaggtcttcaaacccaaaagtagccttgaaacagccggcgtattgccagtaagtttcaagactacttataggttatttattcaactaattagtttaagctttgatgattttcccaactgcaattggtaaagtaacgtgggcatcgtagttcatgacagttccttgaacagtagccggtagcttcaaggtatccggcacaccatgaatataaatgacccttttatcgagctgcaaatcgtcactgccaaaagcggctttaaacgctgtctgcaatttcttcagatcaacgagtttgtcataagcatagtgaccttctgcatcagccactgggtaactgtcatttggcacatgcatcttggctggttcagcgtcaaatggcaccatcgttgtaccggaaaccgattctgtttctggcagatcaacgaaaccgtcaccattagcatcttgtgccgctgtggcgatttctgcaggcttgccatccgggaatccgtggaaatgttcccaatgctgcacattagcaggtgtatcaaacatatcgatatgaatcttcatttgcgcaccatcaatcgtgaacgtcgcggcaccgtgcgctgcgctgccaatcttctcagcgttcaatggcacaatttctgctgtatacttttcagccatgcaaaccactccttttagatttagactcagtttcagcatagtgctaactggaacgactcacaatctattgcatttcaaaagtgaggttgcaagatta

SEQ ID NO: 48 - DNA, 102 bp, Artificial Sequence
  Synthetic sgRNA
cagcattggaacagcgaattgttttagagctagaaatagcaagttaaaataaggctagtccgttatcaacttgaaaaagtggcaccgagtcggtgctttttt

SEQ ID NO: 49 - DNA, 3502 bp, Artificial Sequence
  Synthetic Rep origin #1
ctatcgataagcttaagtgattagtcaaagaatggtgatgacaattgtaaattctatttaatcactttgactagcaaatactaacaacaagacacacacaccaaaaatcaaaaattcactacttttagttaaaaaccacgtaaccacaagaactaatccaatccatgtaatcgggttcttcaaatatttctccaagattttcctcctctaatatgctcaacttaaatgacctattcaataaatctattatgctgctaaatagtttataggacaaataagtatactctaatgacctataaaagatagaaaattaaaaaatcaagtgttcgcttcgctctcactgcccctcgacgttttagtagcctttccctcacttcgttcagtccaagccaactaaaagttttcgggctactctctccttctccccctaataattaattaaaatcttactctgtatatttctgctaatcattcgctaaacagcaaagaaaaaacaaacacgtatcatagatataaatgtaatggcatagtgcgggttttattttcagcctgtatcatagctaaacaaatcgagttgtgtgtccgttttagggcgttctgctagcttgtttaaagtctcttgaatgaatgtatgctctaagtcaaaagaatttgtcagcgcctttatatagctttctttttcttctttttttactttaatgatcgatagcaacaatgatttaacactagcaagttgaatgccaccatttcttcctggtttaatcttaaagaaaatttcctgattcgccttcagtaccttcagcaatttatctaatgtccgttcaggaatgcctagcacttctctaatctcttttttggtcgtcactaaataaggcttgtatacatcgcttttttcgctaatataagccattaaatcttctttccattctgacaaatgaacacgttgacgttcgcttctttttttcttgaatttaaaccacccttgacggacaaataaatctttactggttaaatcacttgatacccaagctttgcaaagaatggtaatgtattccctattagccccttgatagttttctgaataggcacttctaacaattttgattacttctttttcttctaagggttgatctaatcgattattaaactcaaacatattatattcgcacgtttcgattgaatagcctgaactaaagtaggctaaagagagggtaaacatgacgttattacgccctattaaacccttttctcctgaaaatttcgtttcgtgcaataagagattaaaccagggttcatctacttgttttttgccttctgtaccgcttaaaaccgttagacttgaacgagtaaagcccttattatctgtttgtttgaaagaccaatcttgccattctttgaaagaataacggtaattaggatcaaaaaattctacattgtccgttcttggtatgcgagcaataccaaaatgattacacgttagatcaactggcaaagactttccaaaatattctcggatattttgcgaaattattttggctgctttgacagatttaaattctgattttgaagtcacatagactggcgtttctaaaacaaaatatgcttgataacctttatcagatttgataatcatagtaggcataaaacctaaatcaatagcggttgttaaaatatcgcttgctgaaatagtttcttttgccgtgtgaatatcaaaatcaataaagaaggtattgatttgtcttaaattgttttcagaatgtcctttcgtgtatgaacggttttcgtctgcatacgttccataacgataaacgttgggtgtccaatgtgtaaatgtatcttgattttcttgaatcgcttcctcggaagtcagaacaacaccacgaccgccaatcatgcttgattttgagcgatacgcaaaaatagcccctttgcttttacctggcttggtagtgattgagcgaattttactatttttaaatttgtactttaacaagccgtcatgaagcacagtttctacaacaaaagggatattcattcagctgttctcctttcctataaatcctataaaataggttgtttaattaacttggtttgctttttcattcaactgtttcaatattgcatgttttgaaaaagatttttttcctttataagtcaatttttttccactaatcgaataaattattttgttattttctattaacttatatatataatcttccccctccgaagaaaaatacttatctgattttgtttctaagtagatatttctcttttctaactctttcttaaacgtttctagtgtatagatatttgctaattttcttatctccaataaactattttttatataagttttacattcatcatgattcatacaaactccaccttctataaatgaatacaaaaaaagcaatcaaacgatttccgattgattgcttaacaattcttaaattcagtagcttagatacttgaaaactctctgatttccctatataatgatagtacggttatataccgtcttcaaacaaagttaattaaataacttcttacgagggaagagttcatctgactaactgataagcgttggtttggcaatcttatcgggctatgcatttataaaatgtcgtcaaacattttataaatgtgtcatggctcttttttcgtttctattcagttcgttgtttcgttatatctagtataccgcttttaaaaaaaataagcaacgatttcgtgcattattcacacgaagtcattgcttttttcttcttccatttctaaatccaatgttacttgttctgattctgtttctggttctggttctgttggctcatttgggattaaatccactactagcgttgagttagttaactttgcaatttgttctagtgtttttatggttggatctgattttcctgattctattcgtgaataatttgatctactcatttctaattcttggggtaccgccagcatttcggaaaaaaaccacgctaaggattttttctataaaaagagccgttatattaagaataaaacggctcttttatacgtaaaggacgtaaattcatttgcccagtgtcatgtaatccttcaaatttgtattctccaagaaaattgatatgttcccatcctaacggccacgcatatggcattaaatcttctctaaattctcctcttgcttttaattcttctacggctttttccatatatacagtgttccacacacttatagcgttaataattatgtttagtgcactagctctttgtaactggtcttggagagcacgttctctaaattctccacgttgtccaaaaaatataattctagctaatgcattgattgcttctcctttatttaaacctttttgaacccgtctccttacggctttattagatatgtaatccagcgtaaagagggttttctcgattcgtcccatttctccaagtgctgttgcgagtttattttgtcttgcatatgatccgagcttccccatgataa

SEQ ID NO: 50 - DNA, 1695 bp, Artificial Sequence
  Synthetic Rep origin #2
tcagatccttccgtatttagccagtatgttctctagtgtggttcgttgtttttgcgtgagccatgagaacgaaccattgagatcatacttactttgcatgtcactcaaaaattttgcctcaaaactggtgagctgaatttttgcagttaaagcatcgtgtagtgtttttcttagtccgttatgtaggtaggaatctgatgtaatggttgttggtattttgtcaccattcatttttatctggttgttctcaagttcggttacgagatccatttgtctatctagttcaacttggaaaatcaacgtatcagtcgggcggcctcgcttatcaaccaccaatttcatattgctgtaagtgtttaaatctttacttattggtttcaaaacccattggttaagccttttaaactcatggtagttattttcaagcattaacatgaacttaaattcatcaaggctaatctctatatttgccttgtgagttttcttttgtgttagttcttttaataaccactcataaatcctcatagagtatttgttttcaaaagacttaacatgttccagattatattttatgaatttttttaactggaaaagataaggcaatatctcttcactaaaaactaattctaatttttcgcttgagaacttggcatagtttgtccactggaaaatctcaaagcctttaaccaaaggattcctgatttccacagttctcgtcatcagctctctggttgctttagctaatacaccataagcattttccctactgatgttcatcatctgaacgtattggttataagtgaacgataccgtccgttctttccttgtagggttttcaatcgtggggttgagtagtgccacacagcataaaattagcttggtttcatgctccgttaagtcatagcgactaatcgctagttcatttgctttgaaaacaactaattcagacatacatctcaattggtctaggtgattttaatcactataccaattgagatgggctagtcaatgataattactagtccttttcctttgagttgtgggtatctgtaaattctgctagacctttgctggaaaacttgtaaattctgctagaccctctgtaaattccgctagacctttgtgtgttttttttgtttatattcaagtggttataatttatagaataaagaaagaataaaaaaagataaaaagaatagatcccagccctgtgtataactcactactttagtcagttccgcagtattacaaaaggatgtcgcaaacgctgtttgctcctctacaaaacagaccttaaaaccctaaaggcttaagtagcaccctcgcaagctcggttgcggccgcaatcgggcaaatcgctgaatattccttttgtctccgaccatcaggcacctgagtcgctgtctttttcgtgacattcagttcgctgcgctcacggctctggcagtgaatgggggtaaatggcactacaggcgccttttatggattcatgcaaggaaactacccataatacaagaaaagcccgtcacgggcttctcagggcgttttatggcgggtctgctatgtggtgctatctgactttttgctgttcagcagttcctgccctctgattttccagtctgaccacttcggattatcccgtgacaggtcattcagactggctaatgcacccagtaaggcagcggtatcatcaac

SEQ ID NO: 51 - DNA, 158 bp, Artificial Sequence
  Synthetic acrIIA4
atgacggtaacgagtatgtaattagcgaatctgaaaacgagagcattgttgagaaattcatttcagctttcaaaaacggttggaatcaagagtacgaggacgaagaagagttctacaacgatatgcaaaccatcactttgaaaagtgaattgaactaa

SEQ ID NO: 52 - DNA, 738 bp, Artificial Sequence
  Synthetic AbR#1
atgaacaaaaatataaaatattctcaaaactttttaacgagtgaaaaagtactcaaccaaataataaaacaattgaatttaaaagaaaccgataccgtttacgaaattggaacaggtaaagggcatttaacgacgaaactggctaaaataagtaaacaggtaacgtctattgaattagacagtcatctattcaacttatcgtcagaaaaattaaaactgaatactcgtgtcactttaattcaccaagatattctacagtttcaattccctaacaaacagaggtataaaattgttgggagtattccttaccatttaagcacacaaattattaaaaaagtggtttttgaaagccatgcgtctgacatctatctgattgttgaagaaggattctacaagcgtaccttggatattcaccgaacactagggttgctcttgcacactcaagtctcgattcagcaattgcttaagctgccagcggaatgctttcatcctaaaccaaaagtaaacagtgtcttaataaaacttacccgccataccacagatgttccagataaatattggaagctatatacgtactttgtttcaaaatgggtcaatcgagaatatcgtcaactgtttactaaaaatcagtttcatcaagcaatgaaacacgccaaagtaaacaatttaagtaccgttacttatgagcaagtattgtctatttttaatagttatctattatttaacgggaggaaataa

SEQ ID NO: 53 - DNA, 816 bp, E. coli
  misc_feature (1)..(816): AbR#2
atgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaa

SEQ ID NO: 54 - DNA, 2272 bp, E. coli
  misc_feature (1)..(2272): Inducible promoter system #1
cttcaatagagttcttaacgttaatccgaaaaaaactaacgttaatattaaaaaataagatccgcttgtgaattatgtataatttgattagactaaagaataggagaaagtatgatgatatttaaaaaactttctcgttaagataggttgttggtgagcatgttatatacggatgtatcggtttccttaatgcaaaattttgttgctatcttagctcaccaacaacctatcttaatttttctattatatagatatattcaaagaaagataacatttaaacggatcatattagatattttaatagcgattattttttcaatattatatctgtttatttcagatgcgtcattacttgtaatggtattaatgcgattagggtggcattttcatcaacaaaaagaaaataagataaaaacgactgatacagctaatttaattctaattatcgtgatccagttattgttagttgcggttgggactattattagtcagtttaccatatcgattatcaaaagtgatttcagccaaaatatattgaacaatagtgcaacagatataactttattaggtattttctttgctgttttatttgacggcttgttctttatattattgaagaataagcggactgaattacaacatttaaatcaagaaatcattgaattttcgttagaaaaacaatattttatatttatatttattttatttatagtaatagaaattattttagcagttgggaatcttcaaggagtaacagccacgatattattaaccattatcattattttttgtgtccttatcgggatgactttttggcaagtgatgctttttttgaaggcttattcgattcgccaagaagccaatgaccaattggtccggaatcaacaacttcaagattatctagtcaatatcgaacagcagtacaccgaattacggcgatttaagcatgattatcaaaacatcttattatcgttggagagttttgccgaaaagggcgatcagcaacagtttaaggcgtattaccaagaattattagcacaacggccaattcaaagtgaaatccaaggggcagtcattgcacaactcgactacttgaaaaatgatcctattcgaggattagtcattcaaaagtttttggcagccaaacaggctggtgttactttaaaattcgaaatgaccgaaccaatcgaattagcaaccgctaatctattaacggttattcggattatcggtattttattagacaatgcgattgaacaagccgttcaagaaaccgatcaattggtgagttgtgctttcttacaatctgatggtttaatcgaaattacgattgaaaatacggccagtcaagttaagaatctccaagcattttcagagttaggctattcaacgaaaggcgctggtcgggggactggtttagctaatgtgcaggatttgattgccaaacaaaccaatttattcttagaaacacagattgaaaatagaaagttacgacagacattgatgattacggaggaaacttaatttgtatcccgtttatttattagaggatgatttacagcaacaagcgatttatcagcaaattatcgcgaatacgattatgattaacgaatttgcaatgactttaacatgcgctgccagtgatactgagacattgttggcggcaattaaggatcagcaacgaggtttattctttttggatatggaaattgaggataaccgccaagccggtttagaagtggcaactaagattcggcagatgatgccgtttgcgcaaattgtcttcattacaacccacgaggaactgacattattaacgttagaacgaaaaatagcgcctttagattacattctcaaggaccaaacaatggctgaaatcaaaaggcaattgattgatgatctattgttagctgagaagcaaaacgaggcggcagcgtatcaccgagaaaatttatttagttataaaataggtcctcgctttttctcattaccattaaaggaagttgtttatttatatactgaaaaagaaaatccgggtcatattaatttgttagccgttaccagaaaggttacttttccaggaaatttaaatgcgctggaagcccaatatccaatgctctttcggtgtgataaaagttacttagttaacctatctaatattgccaattatgacagtaaaacacggagtttaaaatttgtagatggcagtgaggcaaaagtctcgttccggaaatcacgggaactagtggccaaattaaaacaaatgatgtag

SEQ ID NO: 55 - DNA, 416 bp, Lactobacillus sake
  misc_feature (1)..(416): PorfX promoter
acgccaaatgatcccagtaaaaagccacccgcatggcgggtggctttttattagccctagaagggcttcccacacgcatttcagcgccttagtgccttagtttgtgaatcataggtggtatagtcccgaaatacccgtctaaggaattgtcagataggcctaatgactggcttttataatatgagataatgccgactgtactttttacagtcggttttctaatgtcactaacctgccccgttagttgaagaaggtttttatattacagctccagatctaccggtgggcccatattaacgtttaaccgataaagttgaacgttaatattttttttgcgcagaaatggtaaattgaagcataatagtcttgtaaggtatttagctggctggcgtaaagtatgctttataaaataatat

SEQ ID NO: 56 - DNA, 5 bp, Artificial Sequence
  Synthetic RBS
aggag

SEQ ID NO: 57 - DNA, 2161 bp, Lactococcus lactis
  misc_feature (1)..(2161): Inducible promoter system #2
gaattccccggctttaggtatagtgtgtatctcaatccttggtatattgaaaagaaagactaaaaattgatagattatatttcttcagaatgaatggtataatgaagtaatgagtactaaacaatcggaggtaaagtggtgtataaaattttaatagttgatgatgatcaggaaattttaaaattaatgaagacagcattagaaatgagaaactatgaagttgcgacgcatcaaaacatttcacttcccttggatattactgattttcagggatttgatttgattttgttagatatcatgatgtcaaatattgaagggacagaaatttgtaaaaggattcgcagagaaatatcaactccaattatctttgttagtgcgaaagatacagaagaggatattataaacggcttaggtattggtggggatgactatattactaagccttttagccttaaacagttggttgcaaaagtggaagcaaatataaagcgagaggaacgcaataaacatgcagttcatgttttttcagagattcgtagagatttaggaccaattacattttatttagaagaaaggcgagtctgtgtcaatggtcaaacaattccactgacttgtcgtgaatacgatattcttgaattactatcacaacgaacttctaaagtttatacgagagaggatatttatgatgacgtatatgatgaatattctaatgcactttttcggtcaatctcggagtatatttatcagattaggagtaagtttgcaccatacgatattaatccgataaaaacggttcggggacttgggtatcagtggcatgggtaaaaaatattcaatgcgtcgacggatatggcaagctgtcattgaaattatcataggtacttgtctacttatcctgttgttactgggcttgactttctttctacgacaaattggacaaatcagtggttcagaaactattcgtttatctttagattcagataatttaactatttctgatatcgaacgtgatatgaaacactacccatatgattatattatttttgacaatgatacaagtaaaattttgggaggacattatgtcaagtcggatgtacctagttttgtagcttcaaaacagtcttcacataatattacagaaggagaaattacttatacttattcaagcaataagcatttttcagttgttttaagacaaaacagtatgcctgaatttacaaatcatacgcttcgttcaatttcttataatcaatttacttaccttttcttttttcttggtgaaataatactcattattttttctgtctatcatctcattagagaattttctaagaattttcaagccgttcaaaagattgcattgaagatgggggaaataactacttttcctgaacaagaggaatcaaaaattattgaatttgatcaggttctgaataacttatattcgaaaagtaaggagttagctttccttattgaagcggagcgtcatgaaaaacatgatttatccttccaggttgctgcactttcacatgatgttaagacacctttaacagtattaaaaggaaatattgaactgctagagatgactgaagtaaatgaacaacaagctgattttattgagtcaatgaaaaatagtttgactgtttttgacaagtattttaacacaatgattagttatacaaaacttttgaatgatgaaaatgattacaaagcgacaatctccctggaggattttttgatagatttatcagttgagttggaagagttgtcaacaacttatcaagtggattatcagctagttaaaaaaacagatttaaccactttttacggaaatacattagctttaagtcgagcacttatcaatatctttgttaatgcctgtcagtatgctaaagagggtgaaaaaatagtcagtttgagtatttatgatgatgaaaaatatctctattttgaaatctggaataatggtcatcctttttctgaacaagcaaaaaaaaatgctggaaaactatttttcacagaagatactggacgtagtgggaaacactatgggattggactatcttttgctcaaggtgtagctttaaaacatcaaggaaacttaattctcagtaatcctcaaaaaggtggggcagaagttatcctaaaaataaaaaagtaa

SEQ ID NO: 58 - DNA, 309 bp, Lactococcus lactis
  misc_feature (1)..(309): PnisA promoter
ctcctgttttacaaccgggtgtacatagcgaaatacttgtaatgcgtggtgatgcacctgaatctttcttcgaaacagataccaaatccaagctaaaatcttttgtactcattttgagtgcctccttataatttattttgtagttccttcgaacgaaatcattgtatctaacaaacttcagaatttaatcagagccgtttattatgctcgcgttatcgacaataatattattaccaatactttctcaagatagaattaagactgttttagatttgttaatgtttctattgtcagtatagttataagact

SEQ ID NO: 59 - DNA, 2071 bp, Lactobacillus phage PLE3
  misc_feature (1)..(2071): Recombinase
atgaccatgcttgattacaacacagcggttctgaatgaatatcaacggcgagaacgacttgaagataaagccattgccgattgggagtcctatcacggtaccgtcttgcccaaagatatggatatcgaacaagcggaggagttcttggccaccgccgatgaatatgaagttgatacaaagaaaccttggctctatcaaagctgtgctgcatcgcgttatgagggcgcctttaacaaagacaaagcgaaggaatacttgaaagattggatcaacattcacggccctgagcgattcttaaaagacgctgctagttctacgtatccgaaaacagaactggttgagattttcttcggcggtgacagcttagacgttattgatttcatgaagaatcaaggatttcaggaatggaaataggaggagtagcatatgacgacacaatatgacctaaaaaaaatgccagttaagaaactgattgagacgcagacgattaagaataagtttgcagcgcttctggacaaacgggcaccacagtttctttcatcaattgccagcgcggtaagccttaatccaagcttagccagagttgatcagttaagtgttatcaactcggccctggtagcagcaacgctcgatcttccggttaacccgagcttgggttttgtctacatcgttccatacaagaaccaggcgcagccacagattggttataaaggctatatccaattagctcaacgatcaggacggtatcagcgcctgactgctttaccaatttatgaagatgagttcaagagctggaacccactaacggaggaacttgagtacacgccgaacttccacgatcgcaaagcaagcgaaaaaccggttggctatgccgcatcgttcaaactgactaacggttttgaaaagatggtctattggacttatcagcaagtcgatgatcatcgcaagcgtttcagcaaatctggtggtagcgcggagcccaagggcgtttggaaagacaactacgaggctatggccctgaagacggtaatcaaatcgctgctgactaagtggggtccaatgacaaccgacatgcaaagtgcggtcagtgccgatgaaaaaccagtcgaagctgatccagaactgaaggatgttacccccgaagatcctaactcgatcgaggatgcacttaacgctcccgctgaacccgtcacaaaatcggaggtgaagccagatgctcttaagccagacattacccacgacccaaatgcaggaaaacaaccagaaatctttgacggtcaacaaggataattattactcgctggataccagtttcaaatatcagtctgctacctggtttaagaagtttctgacatgcgaagcggaagcgatggccgagttgcaaggtaaatggataccaagaggtgatccgactgccttgctggttggaaactatctacacagctattttgaatccaagcaagctcatgagtcttttatcaaaggacacccagagatgttctcaactcgtggatcatcaaaaggacaactgaaagccacgtataaacaagctgatgcgatgattgccacgcttgaagctgatgagaatgttcaacgactttatcagggcgaaaaagaagagatcctgaccggtgatctgtttggggtcgagtggatgggcaagctggactgcttcgactccacaaagtcattctttttggatctaaagaccacacagtcgcttcacaagaagtattggaaaccaggagaacgtcaaccaaccagtttcgttgatgcctataactatcagcttcagatggcggtttatcaggagctggtttaccaaaattacggaacgcgaccacgagccttcatcattgccgtgactaaggaagacgtgcccgaccatgccgtcatcgaagtaccacagtaccgtatggacgaggcactggaagagatccatgacagcaccgaacacgttgaggcggttaaatccggtcaggtgcgtcctcatcgctgtgaggcctgtgattactgcaaggcaactaaacgagtcgccacaattatcagcatggatgagctagtcgagtag

(19) United States
(12) Patent Application Publication
(10) Pub. No.: US 2022/0290120 A1
(43) Pub. Date: Sep. 15, 2022

(54) PLASMIDS FOR GENE EDITING

(71) Applicant: NOVOME BIOTECHNOLOGIES, INC., South San Francisco, CA (US)

(72) Inventors: Joel Berry, Berkeley, CA (US); Jonathan Kotula, Berkeley, CA (US); Agnes Oromi-Bosch, Berkeley, CA (US); McKay Shaw, Berkeley, CA (US); Stephen Smith, Berkeley, CA (US)

(21) Appl. No.: 17/432,753

(22) PCT Filed: Feb. 24, 2020
(86) PCT No.: PCT/US2020/019439

§ 371 Date: Aug. 20, 2021

Related U.S. Application Data
(60) Provisional application No. 62/809,869, filed on Feb. 25, 2019.

(51) Int. Cl. C12N 15/10; C12N 15/70; C12N 15/74

(52) U.S. Cl. CPC C12N 15/102; C12N 15/70; C12N 15/746; C12N 2310/20

(57) ABSTRACT

The present invention pertains to single plasmid systems comprising sequences encoding programmable proteins, one or more guides, optionally donor polynucleotides, and optionally anti-CRISPR molecules, for gene editing. These plasmid systems allow for genomic engineering of bacterial strains that are difficult to transform and increase the efficiency of genomic engineering in tractable strains. Additionally, the single plasmids can be configured to provide for the transformation of a number of different bacterial strains using the same plasmid.

### CROSS-REFERENCE TO RELATED APPLICATION

This application claims the benefit under 35 U.S.C. § 119(e)(1) to U.S. Provisional Application No. 62/809,869, filed Feb. 25, 2019, which application is incorporated herein by reference in its entirety.

### TECHNICAL FIELD

The present invention relates to genome editing techniques. More particularly, the invention is directed to the use of single plasmid systems configured to allow efficient genome editing of multiple bacterial species.

### SEQUENCE LISTING

The sequences referred to herein are listed in the Sequence Listing submitted as an ASCII text file entitled “CBI035 30_ST25.txt”-84 KB and was created on 21 Feb. 2020. The Sequence Listing entitled “CBI035 30_ST25.txt” is incorporated herein by reference in its entirety.

### BACKGROUND OF THE INVENTION

Genome editing is commonly used to manipulate, modify, and/or recombine DNA or other nucleic acid molecules in order to modify the genome of an organism. Early strategies for genome modification in prokaryotes made use of endogenous DNA repair enzymes, such as RecA and RecBCD. RecBCD is activated by, and recruited to, double-stranded DNA (dsDNA) breaks when dsDNA breaks are encountered by the DNA replication machinery. RecBCD degrades DNA in a double-stranded manner starting at the dsDNA break then proceeds as a single-stranded DNA (ssDNA) nuclease after encountering a chi site. RecA binds to the newly generated single-stranded DNA and promotes homologous recombination if there is homologous DNA available.

Researchers have taken advantage of this system by transforming bacteria with plasmids that contain a 1 kilobase (kb) stretch of a homologous DNA sequence flanking the desired genomic change. However, this process lacks efficiency because a dsDNA break has to naturally occur near the desired site of homologous recombination, and a double crossover event needs to occur between the genome and the supplied plasmid. Additionally, preparing plasmids with large homology arms is labor intensive, and single crossover events can happen that result in the entire plasmid being incorporated into the genome.

The discovery and implementation of enzymes from the Escherichia coli bacteriophage lambda, termed “lambda RED recombineering,” has greatly increased the efficiency of bacterial genome engineering (see, e.g., Court, et al., Annual Review of Genetics (2002) 36:361-388). As explained in Court, et al., Lambda RED recombineering requires that cells be transformed by a plasmid containing lambda RED recombination enzymes, as well as linear dsDNA homologous to the bacterial genome at the targeted genomic change. The lambda RED enzymes are gam, exo, and beta. Gam inhibits the endogenous recombination enzyme RecBCD that is also a highly potent and processive dsDNA exonuclease. Exo is a DNA exonuclease that generates single-stranded DNA overhangs from the supplied linear dsDNA. Beta binds to single-stranded DNA, and promotes strand invasion and homologous recombination (see, e.g., Court, et al.; Sawitzke, et al., Methods Enzymol. (2013) 533:157-177). As explained in Court, et al., and Sawitzke, et al., beta only requires 30-100 bases of homology for efficient recombination. Therefore, linear dsDNA for recombination can be generated by polymerase chain reaction (PCR) with primers that contain homologous DNA. Lambda RED recombineering greatly increases the efficiency of recombination, but still requires the inclusion of antibiotic resistance genes for selection.

Subsequent work on lambda RED recombineering has shown that beta is most efficient when supplied with linear ssDNA rather than dsDNA (see, e.g., Datta, et al., Proc. Nat. Acad. Sci. USA, (2008) 105:1-10; Sawitzke, et al., Methods Enzymol. (2013) 533: 157-177). Additionally, researchers have shown that beta can work in many bacterial species and is not limited to E. coli-related species (see, e.g., Datta, et al.). However, this technique has been limited to genomic knockouts (gene removal), or nucleotide changes, because it has been difficult or impossible to supply ssDNA long enough for gene insertion (approximately 1 kb).

The current state of bacterial genomic engineering requires the use of two plasmids and double-stranded linear DNA (see, e.g., Reisch, et al., Scientific Reports (2015) 5:15096). One plasmid encodes a programmable nuclease, such as a CRISPR-associated (Cas) protein, e.g. Cas9, the other plasmid encodes single-guide RNA (sgRNA) and the lambda RED enzymes, and the linear dsDNA, supplied separately, contains homology to the bacterial genome and the targeted genetic change. Each plasmid and the linear DNA must be transformed into the bacteria sequentially. This works well in genetically tractable strains such as E. coli, but can be particularly challenging in strains difficult to transform, such as bacteria from the Firmicutes phylum (see, e.g., Reisch, et al.).

In many bacteria, enzymes for non-homologous end joining (NHEJ) do not exist. Therefore the only method of genomic repair is through homologous recombination. Targeting a Cas protein, e.g., Cas9, to cleave genomic DNA can result in bacterial cell death unless homologous recombination can occur. Researchers have shown that lambda RED recombination efficiencies can be improved by targeting Cas9 cleavage to a DNA sequence that would be removed if lambda RED recombination was successful. In that case, organisms that do not perform lambda RED recombination are killed by Cas9 cleavage. Using this system, antibiotic selection is no longer necessary, and successful recombinants can be detected by screening approximately 8-16 colonies via colony PCR (see, e.g., Reisch, et al., Scientific Reports (2015) 5:15096). However, this method requires three transformations and thus is inefficient, even in E. coli. Additionally, three transformations may be impossible to perform in other bacterial strains.

Accordingly, additional methods for increasing gene editing efficiency are highly desirable.

### SUMMARY

The present invention pertains to single plasmid systems comprising sequences encoding programmable proteins, one or more guides, optionally donor polynucleotides, and optionally anti-CRISPR molecules, for gene editing. Unlike the systems described above, the single plasmid systems described herein provide genomic engineering of bacterial strains that are difficult to transform and increase the efficiency of genomic engineering in tractable strains. Additionally, plasmid configurations as described herein allow for the transformation of a number of different bacterial strains using the same plasmid.

Accordingly, in one embodiment, a plasmid is provided. The plasmid comprises: a sequence encoding a programmable CRISPR-associated (Cas) protein operably linked to an inducible promoter; a guide polynucleotide capable of forming a complex with the Cas protein upon expression of the Cas protein, wherein the complex is capable of targeting a selected target site; a first polynucleotide sequence homologous to a 3′ region adjacent to the selected target site; a second polynucleotide sequence homologous to a 5′ region adjacent to the selected target site; a sequence for a selectable marker; and control elements that provide for expression of the plasmid sequences in a selected host cell. In certain embodiments, the first polynucleotide sequence and second polynucleotide sequence are operably linked 5′ and 3′, respectively, to a donor polynucleotide.

In certain embodiments, the Cas protein comprises a catalytically active Cas, e.g., a Cas9 endonuclease capable of producing a double-strand break at the selected target site. In some embodiments, the programmable Cas protein comprises a nickase capable of producing a single-strand break at the selected target site, e.g., a Cas9 nickase (nCas9). In other embodiments, the programmable Cas protein comprises a catalytically inactive Cas protein (dCas) capable of binding to the selected target site but incapable of producing a double-strand or single-strand break at the selected target site, e.g., a dCas9.

In any of the embodiments, the plasmid can further comprise a sequence encoding an anti-CRISPR molecule operably linked to a promoter, wherein the anti-CRISPR molecule is capable of inhibiting the function of the programmable Cas protein. In certain embodiments, the anti-CRISPR molecule is selected from the group consisting of an AcrIIA1, an AcrIIA1-2, an AcrIIA2, an AcrIIA4, and an AcrIIA5. In additional embodiments, a constitutive promoter is operably linked to the sequence encoding the anti-CRISPR molecule.

In additional embodiments, an inducible promoter is operably linked to the sequence encoding the programmable Cas protein, such as an inducible tetracycline promoter.

In further embodiments, the sequence for the selectable marker in the plasmid is capable of imparting antibiotic resistance to the host cell transformed with the plasmid.

In yet additional embodiments, a plasmid is provided that comprises an element organization selected from an element organization as depicted in FIG. 1; an element organization as depicted in FIG. 2; an element organization as depicted in FIG. 3; an element organization as depicted in FIG. 4; an element organization as depicted in FIG. 5; an element organization as depicted in FIG. 6; an element organization as depicted in FIG. 7; an element organization as depicted in FIG. 8; an element organization as depicted in FIG. 9; or an element organization as depicted in FIG. 10.

In certain embodiments, Element 2 of the plasmid comprises a gene encoding a Cas9, a nCas9, or a dCas9.

In other embodiments, Element 7, if present, comprises an anti-CRISPR selected from the group consisting of an AcrIIA1, an AcrIIA1-2, an AcrIIA2, an AcrIIA4, and an AcrIIA5.

In some embodiments, the plasmid comprises two or more single-guide RNAs (sgRNAs),

and/or two or more antibiotic resistance genes, and/or two or more origins of replication.

In yet additional embodiments, a prokaryotic host cell is provided that is transformed with any one of the plasmids described herein. In certain embodiments, the prokaryotic host cell is a Proteobacteria cell, e.g., an Escherichia coli cell; a Bacteroidetes cell, e.g., a Bacteroides spp. cell, such as a Bacteroides thetaiotaomicron cell; or a Firmicutes cell, e.g., a Lactobacillus spp. cell, such as a Lactobacillus casei cell.

In further embodiments, a method for editing a prokaryotic genome is provided. The method comprises: transforming a selected prokaryotic cell, such as a prokaryotic cell described above, with a plasmid described herein; and culturing the cell under conditions whereby the components of the plasmid are expressed such that homologous recombination at the selected target site occurs, thereby editing the prokaryotic genome.

In certain embodiments, the prokaryotic cell is transformed by electroporation, chemical transformation, or conjugation.

These aspects and other embodiments of the invention will readily occur to those of ordinary skill in the art in view of the disclosure herein.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

FIG. 1 is a diagram of a plasmid comprising Plasmid Element Organization A (not to scale). The various elements of the plasmid are numbered and described in detail herein.

FIG. 2 is a diagram of a plasmid comprising Plasmid Element Organization B (not to scale). The various elements of the plasmid are numbered and described in detail herein.

FIG. 3 is a diagram of a plasmid comprising Plasmid Element Organization C (not to scale). The various elements of the plasmid are numbered and described in detail herein.

FIG. 4 is a diagram of a plasmid comprising Plasmid Element Organization D (not to scale). The various elements of the plasmid are numbered and described in detail herein.

FIG. 5 is a diagram of a plasmid comprising Plasmid Element Organization E (not to scale). The various elements of the plasmid are numbered and described in detail herein.

FIG. 6 is a diagram of a plasmid comprising Plasmid Element Organization F (not to scale). The various elements of the plasmid are numbered and described in detail herein.

FIG. 7 is a diagram of a plasmid comprising Plasmid Element Organization G (not to scale). The various elements of the plasmid are numbered and described in detail herein.

FIG. 8 is a diagram of a plasmid comprising Plasmid Element Organization H (not to scale). The various elements of the plasmid are numbered and described in detail herein.

FIG. 9 is a diagram of a plasmid comprising Plasmid Element Organization I (not to scale). The various elements of the plasmid are numbered and described in detail herein.

FIG. 10 is a diagram of a plasmid comprising Plasmid Element Organization J (not to scale). The various elements of the plasmid are numbered and described in detail herein.

FIG. 11 is a summary of multiple experiments grouped together to show the relative efficacy of similar Cas9 plasmids. Solid bars represent the mean ratio of survival of cells after cas9 induction and the error bars represent the standard error of the mean. Plasmids 1, 2, and 8 have accompanying non-targeting (NT) guide data and show that, without a targeting guide, Cas9 single plasmids do not reduce survival of strains, while the presence of a targeting guide results in a range of reduced cell survival.

FIG. 12 is a summary of biological and technical replicate experiments of a Cas9 single plasmid, Plasmid 1, with the ability to induce-site specific recombination in cells. The solid bar represents the mean percent of edited cells after cas9 induction and the error bar represents the standard error of the mean.

FIG. 13 is a summary of biological and technical replicate experiments grouped together to show relative editing efficiency of Cas9 nickase (nCas9) plasmids with and without anti-CRISPR. The solid bars represent the mean percent of edited cells and the error bars represent the standard error of the mean. The same three sgRNA units were tested in Plasmid 4 and Plasmid 5.

FIG. 14 is a summary of biological and technical replicate experiments grouped together to show relative repression efficacy of catalytically inactive Cas9 (dCas9) plasmids with and without anti-CRISPR. Bars represent the mean percent of enzyme expression and the error bars represent the standard error of the mean. The same sgRNA units were tested in Plasmid 6, Plasmid 7, and in the positive control. The negative control utilized a NT guide unit.

FIG. 15 is a summary of multiple experiments grouped together to show relative efficacy of similar Cas9 plasmids. Bars represent the mean conjugation efficiency of the cells with (induced) or without (uninduced) cas9 induction and error bars represent the standard error of the mean. Plasmid 9 has accompanying NT guide data and shows that, without a targeting guide, the Cas9 single plasmid does not reduce survival of strains, while presence of a targeting guide results in reduced cell survival.

FIG. 16 is a summary of biological replicate experiments of a Cas9 single plasmid capable of inducing site-specific recombination in cells. The bar represents the mean percent of edited cells after cas9 induction and the error bar represent the standard error of the mean.

### DETAILED DESCRIPTION OF THE INVENTION

It is to be understood that the terminology used herein is for the purpose of describing particular embodiments only and is not intended to be limiting. As used in this specification and the appended claims, the singular forms “a,” “an,” and “the” include plural referents unless the context clearly dictates otherwise. Thus, for example, reference to “a sgRNA” includes one or more sgRNAs, reference to “a mutation” includes one or more mutations, and the like. It is also to be understood that when reference is made to an embodiment using a sgRNA to target Cas9 to a target site, one skilled in the art can use alternative embodiments of the invention based on the use of other guide polynucleotides in place of the sgRNA.

Unless defined otherwise, all technical and scientific terms used herein have the same meaning as commonly understood by one of ordinary skill in the art to which the invention pertains. Although other methods and materials similar, or equivalent, to those described herein can be used in the practice of the present invention, preferred materials and methods are described herein.

In view of the teachings of the present specification, one of ordinary skill in the art can apply conventional techniques of immunology, biochemistry, chemistry, molecular biology, microbiology, cell biology, genomics, and recombinant polynucleotides, as taught, for example, by the following standard texts: Antibodies: A Laboratory Manual, Second edition, E. A. Greenfield, 2014, Cold Spring Harbor Laboratory Press, ISBN 978-1-936113-81-1; Culture of Animal Cells: A Manual of Basic Technique and Specialized Applications, 7th Edition, R. I. Freshney, 2016, Wiley-Blackwell, ISBN 978-1-118-87365-6; Methods in Molecular Biology (Series), J. M. Walker, ISSN 1064-3745, Humana Press; RNA: A Laboratory Manual, 2010, D. C. Rio, et al., Cold Spring Harbor Laboratory Press, ISBN 978-0879698911; Methods in Enzymology (Series), Academic Press; Molecular Cloning: A Laboratory Manual (Fourth Edition), 2012, M. R. Green, et al., Cold Spring Harbor Laboratory Press, ISBN 978-1605500560; Bioconjugate Techniques, Third Edition, 2013, G. T. Hermanson, Academic Press, ISBN 978-0123822390.

Programmable nucleases enable targeted genetic modifications in a host cell genome by creating site-specific breaks at desired locations in the genome. Such nucleases include, but are not limited to, Clustered Regularly Interspaced Short Palindromic Repeats (CRISPR)-associated (Cas) nucleases, zinc finger nucleases (ZFNs), transcription activator-like effector nucleases (TALENs), meganucleases, and MegaTALs.

Cas nucleases (also termed “Cas enzymes” and “Cas proteins” herein) comprise programmable adaptive immune systems of bacterial and archaeal origin. CRISPR-Cas systems are classified into two distinct classes, Class 1 and Class 2, described in detail in Koonin, et al., Curr Opin Microbiol. (2017) 37:67-78; and Yan, et al., Science (2019) 363:88-91. By a “CRISPR-Cas system,” as used herein, is meant any of the various CRISPR-Cas classes, types and subtypes. By a “programmable CRISPR protein,” “programmable CRISPR enzyme,” and “programmable CRISPR endonuclease” as used herein is meant a molecule derived from a CRISPR-Cas system and that is capable of creating a site-specific double-strand break (in the case of a catalytically active enzyme); a single-strand break (in the case of a nickase); or molecule that binds to a target site but does not cleave at the site (in the case of a catalytically inactive molecule).

CRISPR Class 1 systems comprise a multiprotein effector complex (Type I (Cascade effector complex), III (Cmr/Csm effector complex), and IV); and CRISPR Class 2 systems comprise a single effector protein (Type II (Cas9)), V (Cas12a, previously referred to as Cpf1), and VI (Cas13a, previously referred to as C2c2)).

CRISPR Class 1 Type I and Type III systems typically encode proteins that combine with a CRISPR RNA (crRNA or “guide RNA”) to form a Cascade complex or Cmr/Csm complex, respectively. These complexes comprise multiple proteins and a crRNA, which are transcribed from this CRISPR locus. In Type I and Type III CRISPR-Cas systems, primary processing of a pre-crRNA is catalyzed by Cash. This typically results in a crRNA with a 5′ handle of 8 nucleotides, a spacer region, and a 3′ handle; both 5′ and 3′ handles are derived from the repeat sequence. In some subtypes, the 3′ handle forms a stem-loop structure; in other systems, secondary processing of the 3′ end of crRNA is catalyzed by ribonuclease(s) (see, e.g., van der Oost, et al., Nature Reviews Microbiology (2014) 12:479-492.

CRISPR Class 2 Type II, Type V, and Type VI systems comprise a single-subunit protein (e.g., Cas9, Cas12a, Cas12b (C2c1), C2c4, C2c5, Cas13a (C2c2), Cas13b (C2c6), Cas13c (C2c7) protein) that forms an effector complex with guide RNA.

Class 2 Type II CRISPR systems comprise a Cas9 protein encoded by the cas9 gene and a cognate guide RNA. The cognate guide RNA comprises the crRNA and a trans-activating CRISPR RNA (tracrRNA). Ran, et al., Nature (2015) 520:186-191 present the crRNA/tracrRNA sequences and secondary structures of eight Type II CRISPR-Cas9 systems. Additionally, Fonfara, et al., Nucleic Acids Research (2014) 42:2577-2590 present the crRNA/tracrRNA sequences and secondary structures of eight Type II CRISPR-Cas9 systems. See also PCT Publication No. WO 2013/176772, published Nov. 28, 2013; PCT Publication No. WO 2014/023828, published Feb. 19, 2015 (each of each of which is incorporated herein by reference in its entirety).

The adaptive immunity mechanism of action in the Class 1 Type I and Type III CRISPR-Cas systems involves essentially three phases: adaptation, expression, and interference. In the adaptation phase, a foreign DNA or RNA infects the host and proteins encoded by various cas genes bind regions of the infecting DNA or RNA. Such regions are called protospacers. A protospacer adjacent motif (PAM) is a short nucleotide sequence (e.g., 2- to 6-bp DNA sequence) that is adjacent to the protospacer. For most CRISPR systems, the PAM nucleotide sequence serves as recognition motif for the nuclease.

In Type II systems, nucleic acid target sequence recognition, binding, and cleavage involves Cas9 protein, crRNA, and tracrRNA. The RuvC-like nuclease (RNase H fold) domain and the HNH (McrA-like) nuclease domain of the Cas9 protein each cleave one of the strands of the double-stranded nucleic acid target sequence. The Cas9 protein cleavage activity of Type II systems also requires hybridization of crRNA to tracrRNA to form a duplex that facilitates the crRNA and nucleic acid target sequence binding by the Cas9 protein. For a Cas9 protein/tracrRNA/crRNA complex to cleave a double-stranded DNA target sequence, the DNA target sequence is adjacent to a cognate PAM. By engineering a crRNA to have an appropriate spacer sequence, the complex can be targeted to cleave at a locus of interest, e.g., a locus at which sequence modification is desired.

As used herein, “a Cas protein” (such as “a Cas9 protein,” “a Cas13 protein,” “a Cas12 protein,” etc.), refers to a Cas protein derived from any species, subspecies, or strain of bacteria that encodes the Cas protein of interest, as well as variants and orthologs of the particular Cas protein in question. Non-limiting examples of Cas proteins include Cas 1, Cas1B, Cas2, Cas3, Cas4, Cas5, Cash, Cas7, Cas8, Cas9, Cas10, Cas12a, Cas12d, Cas13d, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, C2C1, C2C2, C2C3, CASCADE, homologs thereof, and modified versions thereof. In some embodiments, the sequence encoding the Cas protein is codon-optimized for expression in a cell of interest. In some embodiments, the Cas protein directs cleavage of one or two strands at the location of the target sequence. In some embodiments, the Cas protein lacks DNA strand cleavage activity. In other embodiments, the Cas protein acts as a nickase. The choice of Cas protein will depend upon the particular conditions of the methods used as described herein.

The term “Cas9 protein,” as used herein refers to wild-type proteins derived from Class 2 Type II CRISPR systems, modifications of the Cas9 proteins, variants of Cas9 proteins, Cas9 orthologs, and combinations thereof. Cas9 proteins can be derived from any of various bacterial species having genomes that encode such proteins. Variants and modifications of Cas9 proteins are known in the art. For example, U.S. Pat. Nos. 9,260,752; 9,410,198; 9,909,122; 9,725,714; 9,803,194; 9,809,814 (each of which is incorporated herein by reference in its entirety) teach a large number of exemplary wild-type Cas9 polypeptides, as well as modifications and variants of Cas9 proteins. Non-limiting examples of Cas9 proteins include Cas9 proteins from Streptococcus pyogenes (GI: 15675041) (SpyCas9); Listeria innocua Clip 11262 (GI: 16801805); Streptococcus mutans UA159 (GI: 24379809); Streptococcus thermophilus LIVID-9 (S. thermophilus A, GI: 11662823; S. thermophilus B, GI: 116627542); Lactobacillus buchneri NRRL B-30929 (GI: 331702228); Treponema denticola ATCC 35405 (GI: 42525843); Francisella novicida U112 (GI: 1*18497352); Campylobacter jejuni subsp. Jejuni NCTC 11168 (GI: 218563121); Pasteurella multocida subsp. multocida str. Pm70 (GI: 218767588); Neisseria meningitidis Zs491 (GI: 15602992); Actinomyces naeslundii (GI: 489880078).

By “dCas protein” is meant a nuclease-deactivated Cas protein, also termed “catalytically inactive,” “catalytically dead,” or “dead Cas protein.” Such molecules lack all or a portion of endonuclease activity and can therefore be used to regulate genes in an RNA-guided manner (see, e.g., Jinek, et al., Science (2012) 337:816-821). This is accomplished by introducing mutations that inactivate Cas nuclease function. For Cas9, this can be done by mutating both of the two catalytic residues (D10A in the RuvC-1 domain, and H840A in the HNH domain, numbered relative to SpyCas9) of the gene encoding Cas9. These mutations to SpyCas9 completely inactivate both the nuclease and nickase activities. It is understood that mutation of other catalytic residues to reduce activity of either or both of the nuclease domains can also be carried out by one skilled in the art. In doing so, dCas9 is unable to cleave dsDNA but retains the ability to sequence-specifically bind DNA. Targeting specificity is determined by complementary base-pairing of a single-guide RNA to the genomic locus and the PAM.

By “nCas,” as used herein, is meant a Cas nickase that maintains the ability to bind to and make a single-strand break at a target site. In the case of “nCas9,” the molecule will typically include a mutation in one, but not both of the Cas9 endonuclease domains (HNH and RuvC).

“Cas12a,” previously referred to as “Cpf1,” refers to a CRISPR-Cas RNA-guided DNA endonuclease found in CRISPR Type V systems. The PAM for Cas12a is a “TTN” motif located 5′ to its protospacer target, as opposed to a 3′ “NGG” PAM motif used by Cas9. Cas12a binds a crRNA that carries the protospacer sequence for base-pairing to the target. Unlike Cas9, Cas12a does not require a separate tracrRNA and is devoid of a tracrRNA gene at the Cas12a-CRISPR locus. Thus, Cas12a only requires a crRNA that is approximately 43 nucleotides (nt) in length, 24 nucleotides (nt) of which are the protospacer and 19 nt the constitutive direct repeat sequence. Cas12a appears to be directly responsible for cleaving the 43 base crRNAs apart from the primary transcript (see, e.g., Fonfara, et al., Nature (2016) 532:517-521).

The term “CASCADE” refers to a CRISPR Type I multiprotein complex known as “CRISPR-associated complex for antiviral defense.” For a description of the CASCADE complex, see, e.g., Jore, et al., Nature Structural and Molecular Biology (2011) 18:529-536. Modified CASCADE systems are described in, e.g., U.S. Pat. Nos. 9,885,026; 10,435,678, 10,227,576; 10,329,547; 10,457,922; PCT Publication No, WO 2019/241542, published Dec. 19, 2019 (each of which is incorporated herein by reference in its entirety).

As used herein, “dual-guide RNA” refers to a two-component RNA component capable of associating with a Class 2 Type II Cas9 protein. A representative CRISPR Class 2 Type II CRISPR-Cas-associated dual-guide RNA includes a Cas-crRNA and Cas-tracrRNA, paired by hydrogen bonds to form secondary structure (see, e.g., Jinek, et al., Science (2012) 337:816-21). A Cas-dual-guide RNA is capable of forming a nucleoprotein complex with a cognate Cas9 protein, wherein the complex is capable of targeting a nucleic acid target sequence complementary to the spacer sequence.

As used herein, “single-guide RNA” (sgRNA) refers to a single, contiguous RNA sequence that interacts with a cognate Cas9 protein essentially as described for tracrRNA/crRNA polynucleotides. A Cas9 single-guide RNA (Cas9-sgRNA) is a guide RNA wherein the Cas9-crRNA is covalently joined to the Cas9-tracrRNA, often through a tetraloop, and forms a RNA polynucleotide secondary structure through base-pair hydrogen bonding. See, e.g., Jinek, et al., Science (2012) 337:816-821; PCT Publication No. WO 2013/176772, published Nov. 28, 2013; (each of which is incorporated herein by reference in its entirety).

A “guide polynucleotide” refers to one or more polynucleotides that guide a protein, such as a Cas nuclease, a dCas nuclease, or a nCas nuclease, to preferentially target a nucleic acid target sequence present in a polynucleotide (relative to a polynucleotide that does not comprise the nucleic acid target sequence). Guide polynucleotides can comprise ribonucleotide bases (e.g., RNA); deoxyribonucleotide bases (e.g., DNA); combinations of ribonucleotide bases and deoxyribonucleotide bases (e.g., RNA/DNA chimeric molecules) such as single-guide and dual-guide RNA/DNA chimeric molecules (chRDNAs) (see, e.g., U.S. Pat. Nos. 9,580,701; 9,650,617; 9,688,972; 9,771,601; 9,868,962; 10,519,468 (each of which is incorporated herein by reference in its entirety)); nucleotides; nucleotide analogs; modified nucleotides; and the like; as well as synthetic, naturally occurring, and non-naturally occurring modified backbone residues or linkages. Thus, a guide polynucleotide, as used herein, site-specifically guides a protein, such as Cas9, to a target nucleic acid.

Many guide polynucleotides are known including, but not limited to, sgRNA (including miniature and truncated sgRNAs as described in U.S. Published Patent Application No. 2017/0114334, published Apr. 27, 2017; and U.S. Published Patent Application No. 2017/0051276, published Feb. 23, 2017 (each of which is incorporated herein by reference in its entirety)); alternative CRISPR nucleic acid-targeting Type II nucleic acid scaffolds, including those described in e.g., U.S. Pat. Nos. 9,771,600; 9,970,029; 10,100,333; 9,816,093; 9,677,090; 9,745,562; 9,816,081; 9,957,490; 10,023,853; 10,125,354; 10,138,472 (each of which is incorporated herein by reference in its entirety); dual-guide RNA, including but not limited to, crRNA/tracrRNA molecules; and the like; the use of which depends on the particular Cas protein. Also useful are 2-bit and 3-bit split-nexus guide polynucleotides, such as single-guide and dual-guide sn-Cas polynucleotides, described in e.g., U.S. Pat. Nos. 9,745,600; 9,580,727; 9,970,026; 9,970,027 (each of which is incorporated herein by reference in its entirety). For a non-limiting description of other exemplary guide polynucleotides, see, e.g., PCT Publication No. WO 2014/150624, published Sep. 29, 2014; PCT Publication No. WO 2015/200555, published Mar. 10, 2016; PCT Publication No. WO 2016/201155, published Dec. 15, 2016; PCT Publication No. WO 2017/027423, published Feb. 16, 2017; PCT Publication No. WO 2017/070598, published Apr. 27, 2017; PCT Publication No. WO 2016/123230, published Aug. 4, 2016 (each of which is incorporated herein by reference in its entirety).

As used herein, a programmable nuclease (e.g., a Cas9 protein), or a catalytically inactive programmable nuclease (e.g., a dCas9 protein) is said to “target” a polynucleotide if a guide polynucleotide/programmable nuclease complex associates with, binds, and/or cleaves (in the case of a catalytically active programmable nuclease) or binds to but does not cleave (in the case of a catalytically inactive programmable nuclease) a polynucleotide at the nucleic acid target region within the polynucleotide. In certain embodiments, the target region is “in proximity to” a gene coding for a protein, i.e., the target region can be adjacent to, operably linked to, or even within a gene of interest.

As used herein, a “site-directed polypeptide or protein” refers to a polypeptide that recognizes and/or binds to a nucleic acid target sequence or the complement of the nucleic acid target sequence. The site directed polypeptide, alone or in combination with guide polynucleotides, will bind to a nucleic acid target sequence or to the complement of the nucleic acid target sequence.

As used herein, the term “cognate” typically refers to a Cas protein (e.g., Cas9 protein) and one or more polynucleotides (e.g., a CRISPR-Cas9-associated guide polynucleotide) that are capable of forming a nucleoprotein complex capable of site-directed binding to a nucleic acid target sequence complementary to the nucleic acid target binding sequence present in one of the one or more polynucleotides.

As used herein, the terms “complex,” “nucleoprotein complex,” and “guide polynucleotide/Cas complex” refer to complexes comprising a guide polynucleotide and a protein that bind to a nucleic acid target sequence. The Cas protein of the complex can affect a blunt-ended double-strand break, a double-strand break with sticky ends, nick one strand, or perform other functions on the nucleic acid target sequence.

“Transcription activation-like effectors” (TALEs) are DNA binding proteins of bacterial origin. The TAL effector DNA-binding domain recognizes specific individual base pairs (bp) in a target DNA sequence by using a known cipher involving two key amino acid residues, also referred to as the repeat variable di-residues (RVDs). See, e.g., Mussolino, et al., Nucleic Acids Res. (2011) 39:9283-9293. Depending on the TALE protein sequence, TALEs can bind any DNA base (G, T, A, C). A large number of TALEs are known in the art. Several TALE DNA binding domains can be fused together and engineered to bind any contiguous DNA sequence. Typically, about 15 TALE DNA binding domains are fused together to recognize a 15-nucleotide DNA sequence. TALEs can be fused to transcriptional activators and repressors. Engineered TALEs can be used for transcriptional activation or repression in a cell.

“Transcription activation-like effector nucleases” (TALENs) are TALEs that are fused to the DNA-cleaving domain of a restriction enzyme such as FokI. TALENs are engineered to bind and cleave any desired DNA sequence. TALENs are typically used for genome engineering of an organism. See, e.g., Mussolino, et al., Nucleic Acids Res. (2011) 39:9283-9293, for a description of TALENs.

“Meganucleases” or “homing endonucleases” refer to a family of enzymes that recognize, bind, and cleave specific DNA sequences (see, e.g., Stoddard, Mobile DNA (2014) 5:7). The DNA recognition site of meganucleases are typically 12 to 40 bp. A large number of meganucleases are known in the art. Meganucleases can be engineered to bind and cleave any DNA sequence. Meganucleases can also be engineered such that they are catalytically inactive and can bind but not cleave DNA. Meganucleases can be fused to other proteins such as transcriptional activators and repressors or other nucleases. Engineered meganucleases can be used for transcriptional activation or repression or genome engineering of a cell. A “MegaTAL” refers to a hybrid nuclease that includes TAL effector domains fused to a portion of a meganuclease (see, e.g., Boissel, et al., Nucleic Acids Research (2014) 42:2591-2601).

“Zinc fingers” are DNA binding proteins or DNA binding protein domains. The proteins or protein domains are often but not always coordinated with one or more zinc ions that recognize particular DNA sequences. A large number of zinc finger domains and proteins are known in the art (see, e.g., Miller, et al., EMBO J. (1985) 4:1609-1614; Rhodes, et al., Sci. Amer. (1993) February: 56-65; Klug, A., J. Mol. Biol. (1999) 293:215-218). Depending on the zinc finger sequence, one zinc finger domain typically binds a triplet of DNA bases. Several zinc fingers can be fused together and engineered to bind any target DNA sequence. Generally, about 5 zinc finger DNA binding domains are fused together to recognize a 15-nucleotide DNA sequence. Zinc fingers can be fused to transcriptional activators and repressors. Engineered zinc fingers can be used for transcriptional activation or repression in a cell.

“Zinc finger nucleases” (ZFNs) are engineered zinc fingers that are fused with the DNA-cleaving domain of a restriction enzyme such as FokI. ZFNs can be engineered to bind and cleave any target DNA sequence. Engineered ZFNs are typically used for genome engineering of an organism. See, e.g., Carrol et al., Nat. Protoc. (2006) 1:1329-1341; U.S. Pat. Nos. 8,034,598; 7,914,796 (each of which is incorporated herein by reference in its entirety).

By “donor polynucleotide” or “donor PN” is meant a polynucleotide that can be directed to, and inserted into a target site of interest, to modify the target nucleic acid. All or a portion of the donor polynucleotide can be inserted into the target nucleic acid. The donor polynucleotide can be used for repair of the break in the target DNA sequence resulting in the transfer of genetic information (e.g., polynucleotide sequences) from the donor at the site or in close proximity of the break in the DNA (termed “target site” herein). Accordingly, new genetic information (e.g., polynucleotide sequences) may be inserted or copied at a target DNA site. The donor can be used to insert or replace polynucleotide sequences in a target sequence, for example, to introduce a polynucleotide that encodes a protein or functional RNA (e.g., siRNA), to introduce a protein tag, to modify a regulatory sequence of a gene, or to introduce a regulatory sequence to a gene (e.g., a promoter, an enhancer, an internal ribosome entry sequence, a start codon, a stop codon, a localization signal, or polyadenylation signal), to modify a nucleic acid sequence (e.g., introduce a mutation), and the like.

Targeted DNA modifications using donor polynucleotides for large changes (e.g., more than 100 bp insertions or deletions) traditionally use double- or single-stranded donor templates that contain homology arms homologous to sequences flanking the genomic site of alteration. Each arm can vary in length, but is typically longer than about 25 bp, such as longer than 30 bp, such as 30-1500 bp, e.g., 30-1500 bp, such as 30 to 100 . . . 200 . . . 300 . . . 400 . . . 500 . . . 600 . . . 700 . . . 800 . . . 900 . . . 1000 . . . 1500 bp or any integer between these values. However, these numbers can vary, depending on the size of the donor polynucleotide and the target polynucleotide. The sequences that homology arms target upstream and downstream of the genomic site can be directly adjacent to the genomic site of alteration or they can be far apart (such as 1 bp, 10 bp, or even up to several thousand bps). Thus, after successful integration of the donor template, parts of the original genome can be deleted (such as 1 bp, 10 bp, up to several thousand bps). This method can be used to generate large modifications, including genomic deletions, insertions of reporter genes such as fluorescent proteins or antibiotic resistance markers, or metabolic pathway genes, and genomic deletions of reporter genes such as fluorescent proteins or antibiotic resistance markers, or metabolic pathway genes.

For smaller insertions, single-stranded oligonucleotides containing flanking sequences on each side that are homologous to the target region (called “homology arms”) can be used and can be oriented in either the sense or antisense direction relative to the target locus. The length of each arm can vary, but the length of at least one arm is typically longer than about 10 bases, such as from 10-150 bases, e.g., 10 . . . 20 . . . 30 . . . 40 . . . 50 . . . 60 . . . 70 . . . 80 . . . 90 . . . 100 . . . 110 . . . 120 . . . 130 . . . 140 . . . 150, or any integer within these ranges. However, these numbers can vary, depending on the size of the donor polynucleotide and the target polynucleotide. In some embodiments, the length of at least one arm is 10 bases or more. In other embodiments, the length of at least one arm is 20 bases or more. In yet other embodiments, the length of at least one arm is 30 bases or more. In some embodiments, the length of at least one arm is less than 100 bases. In further embodiments, the length of at least one arm is greater than 100 bases. For single-stranded DNA oligonucleotide design, typically an oligonucleotide with up to 100-150 bp total homology is used. The mutation is introduced in the middle, yielding 50-75 bp homology arms for a donor designed to be symmetrical about the target site.

A “genomic region” is a segment of a chromosome in the genome of a host cell that is present on either side of the nucleic acid target sequence site or, alternatively, also includes a portion of the nucleic acid target sequence site. The homology arms of the donor polynucleotide have sufficient homology to undergo homologous recombination with the corresponding genomic regions. In some embodiments, the homology arms of the donor polynucleotide share significant sequence homology to the genomic region immediately flanking the nucleic acid target sequence site; it is recognized that the homology arms can be designed to have sufficient homology to genomic regions farther from the nucleic acid target sequence site.

The terms “engineered,” “genetically engineered,” “genetically modified,” “recombinant,” “modified,” and “non-naturally occurring” indicate intentional human manipulation of the genome of an organism. Methods of genetic modification include, for example, heterologous gene expression, gene or promoter insertion or deletion, nucleic acid mutation, altered gene expression or inactivation, enzyme engineering, directed evolution, knowledge-based design, random mutagenesis methods, gene shuffling, codon optimization, and the like. Methods for genetically engineering organisms are described in detail herein.

“Gene editing” or “genome editing,” as used herein, refers to the insertion, deletion, or replacement of a nucleotide sequence at a specific site in the genome of an organism or cell.

The terms “wild-type,” “naturally occurring,” and “unmodified” are used herein to mean the typical (or most common) form, appearance, phenotype, or strain existing in nature; for example, the typical form of cells, organisms, characteristics, polynucleotides, proteins, macromolecular complexes, genes, RNAs, DNAs, or genomes as they occur in and can be isolated from a source in nature. The wild-type form, appearance, phenotype, or strain serve as the original parent before an intentional modification. Thus, mutant, variant, chimeric, engineered, recombinant, and modified forms are not wild-type forms.

As used herein, the terms “nucleic acid,” “nucleotide sequence,” “oligonucleotide,” and “polynucleotide” are interchangeable. All refer to a polymeric form of nucleotides. The nucleotides may be deoxyribonucleotides (DNA) or ribonucleotides (RNA), or analogs thereof, and they may be of any length. Polynucleotides may perform any function and may have any secondary structure and three-dimensional structure. The terms encompass known analogs of natural nucleotides and nucleotides that are modified in the base, sugar and/or phosphate moieties. Analogs of a particular nucleotide have the same base-pairing specificity (e.g., an analog of A base pairs with T). A polynucleotide may comprise one modified nucleotide or multiple modified nucleotides. Examples of modified nucleotides include methylated nucleotides and nucleotide analogs. Nucleotide structure may be modified before or after a polymer is assembled. Following polymerization, polynucleotides may be additionally modified via, for example, conjugation with a labeling component or target-binding component. A nucleotide sequence may incorporate non-nucleotide components. The terms also encompass nucleic acids comprising modified backbone residues or linkages that are synthetic, naturally occurring, and non-naturally occurring, and have similar binding properties as a reference polynucleotide (e.g., DNA or RNA). Examples of such analogs include, but are not limited to, phosphorothioates, phosphoramidates, methyl phosphonates, chiral-methyl phosphonates, 2-O-methyl ribonucleotides, peptide-nucleic acids (PNAs), and morpholino structures.

Unless noted otherwise, polynucleotide sequences are displayed herein in the conventional 5′ to 3′ orientation.

As used herein, the term “complementarity” refers to the ability of a nucleic acid sequence to form hydrogen bond(s) with another nucleic acid sequence (e.g., through traditional Watson-Crick base pairing). A percent complementarity indicates the percentage of residues in a nucleic acid molecule that can form hydrogen bonds with a second nucleic acid sequence. When two polynucleotide sequences have 100% complementarity, the two sequences are perfectly complementary, i.e., all of a first polynucleotide's contiguous residues hydrogen bond with the same number of contiguous residues in a second polynucleotide.

As used herein, the term “sequence identity” generally refers to the percent identity of bases or amino acids determined by comparing a first polynucleotide or polypeptide to a second polynucleotide or polypeptide using algorithms having various weighting parameters. Sequence identity between two polypeptides or two polynucleotides can be determined using sequence alignment by various methods and computer programs (e.g., BLAST, CS-BLAST, FASTA, HMMER, L-ALIGN, etc.), available through the worldwide web at sites including GENBANK (ncbi.nlm.nih.gov/genbank/) and EMBL-EBI (ebi.ac.uk.). Sequence identity between two polynucleotides or two polypeptide sequences is generally calculated using the standard default parameters of the various methods or computer programs. Generally, the various proteins for use herein will have at least about 75% or more sequence identity to the wild-type or naturally occurring sequence of the protein of interest, such as about 80%, such as about 85%, 90%, 91%, 92%, 93%, 94%, 95%, 96%, 97%, 98%, 99%, or complete identity.

As used herein, “double-strand break” (DSB) refers to both strands of a double-stranded segment of nucleic acid being severed. In some instances, if such a break occurs, one strand can be said to have a “sticky end” wherein nucleotides are exposed and not hydrogen bonded to nucleotides on the other strand. In other instances, a “blunt end” can occur wherein both strands remain fully base-paired with each other.

As used herein, the term “recombination” refers to a process of exchange of genetic information between two polynucleotides.

As used herein, “nucleic acid repair,” such as, but not limited to, DNA repair, encompasses any process whereby cellular machinery repairs damage to a nucleic acid molecule contained in the cell. The damage repaired can include single-strand breaks, double-strand breaks, or mis-incorporation of bases.

As used herein, the term “homology-directed repair” or “HDR” refers to DNA repair that takes place in cells, for example, during repair of double-strand and single-strand breaks in DNA. HDR requires nucleotide sequence homology and can use a “donor template” (donor template DNA, donor polynucleotide, or oligonucleotide, used interchangeably herein) to repair the sequence where the DSB occurred (e.g., DNA target sequence). This results in the transfer of genetic information from, for example, the donor template DNA to the DNA target sequence. HDR may result in alteration of the DNA target sequence (e.g., insertion, deletion, mutation) if the donor template DNA sequence or oligonucleotide sequence differs from the DNA target sequence and part or all of the donor template DNA polynucleotide or oligonucleotide is incorporated into the DNA target sequence. In some embodiments, an entire donor template DNA polynucleotide, a portion of the donor template DNA polynucleotide, or a copy of the donor polynucleotide is copied or integrated at the site of the DNA target sequence.

“Homologous recombination” or “FIR” is the most common type of HDR. In HR, sequences are exchanged between homologous or identical molecules of double-stranded or single-stranded nucleic acids. Most bacteria use a HR repair pathway to repair breaks in their genomes which requires a strand of homologous DNA in order to repair the break. Resynthesis of the damaged region is accomplished using the undamaged molecule as a template. HR can produce new combinations of DNA sequences during cell division. These new combinations of DNA can cause genetic variation in daughter cells. For dsDNA, most forms of HR involve the same basic steps. After a DSB occurs, sections of DNA around the 5′ ends of the break are cut away in a process called resection. Following resection, typically an overhanging 3′ end of the broken DNA molecule then “invades” a similar or identical DNA molecule that is not broken. After strand invasion, the further sequence of events may follow either of two main pathways; the double-strand break repair (DSBR) pathway or the synthesis-dependent strand annealing (SDSA) pathway. For a description of HR see, e.g., Jasin, et al., Cold Spring Harbor Perspect. Biol. (2013) 5:a012740; Court, et al., (2002) 36:361-388; Pardo, et al., Cell Mol. Life Sci. (2009) 66:1039-1056; Shrivastav, et al., Cell Res. (2008) 18:134-147.

The terms “vector” and “plasmid” are used interchangeably and as used herein refer to a polynucleotide vehicle to introduce genetic material into a cell. Vectors can be linear or circular. Vectors can integrate into a target genome of a host cell or replicate independently in a host cell. Vectors can comprise, for example, an origin of replication, a multicloning site, and/or a selectable marker. An expression vector typically comprises an expression cassette. Vectors and plasmids include, but are not limited to, integrating vectors, prokaryotic plasmids, eukaryotic plasmids, plant synthetic chromosomes, episomes, viral vectors, cosmids, and artificial chromosomes.

As used herein the term “expression cassette” is a polynucleotide construct, generated recombinantly or synthetically, comprising regulatory sequences operably linked to a selected polynucleotide to facilitate expression of the selected polynucleotide in a host cell. For example, the regulatory sequences can facilitate transcription of the selected polynucleotide in a host cell, or transcription and translation of the selected polynucleotide in a host cell. An expression cassette can, for example, be integrated in the genome of a host cell or be present in an expression vector.

As used herein, the terms “regulatory sequences,” “regulatory elements,” and “control elements” are interchangeable and refer to polynucleotide sequences that are upstream (5′ non-coding sequences), within, or downstream (3′ non-translated sequences) of a polynucleotide target to be expressed. Regulatory sequences influence, for example, the timing of transcription, amount or level of transcription, RNA processing or stability, and/or translation of the related structural nucleotide sequence. Regulatory sequences may include activator binding sequences, enhancers, origins of replication, introns, polyadenylation recognition sequences, promoters, repressor binding sequences, stem-loop structures, translational initiation sequences, translation leader sequences, transcription termination sequences, translation termination sequences, primer binding sites, and the like.

As used herein the term “operably linked” refers to polynucleotide sequences or amino acid sequences placed into a functional relationship with one another. For instance, a promoter or enhancer is operably linked to a coding sequence if it regulates, or contributes to the modulation of, the transcription of the coding sequence. Operably linked DNA sequences encoding regulatory sequences are typically contiguous to the coding sequence. However, enhancers can function when separated from a promoter by up to several kilobases or more. Additionally, multicistronic constructs can include multiple coding sequences which use only one promoter by including a 2A self-cleaving peptide, an IRES element, etc. Accordingly, some polynucleotide elements may be operably linked but not contiguous.

As used herein, the term “expression” refers to transcription of a polynucleotide from a DNA template, resulting in, for example, an mRNA or other RNA transcript (e.g., non-coding, such as structural or scaffolding RNAs). The term further refers to the process through which transcribed mRNA is translated into peptides, polypeptides, or proteins. Transcripts and encoded polypeptides may be referred to collectively as “gene product.” Expression may include splicing the mRNA in a eukaryotic cell, if the polynucleotide is derived from genomic DNA.

As used herein, the term “amino acid” refers to natural and synthetic (unnatural) amino acids, including amino acid analogs, modified amino acids, peptidomimetics, glycine, and D or L optical isomers.

As used herein, the terms “peptide,” “polypeptide,” and “protein” are interchangeable and refer to polymers of amino acids. A polypeptide may be of any length. It may be branched or linear, it may be interrupted by non-amino acids, and it may comprise modified amino acids. The terms may be used to refer to an amino acid polymer that has been modified through, for example, acetylation, disulfide bond formation, glycosylation, lipidation, phosphorylation, cross-linking, and/or conjugation (e.g., with a labeling component or ligand). Polypeptide sequences are displayed herein in the conventional N-terminal to C-terminal orientation.

Polypeptides and polynucleotides can be made using routine techniques in the field of molecular biology. Furthermore, essentially any polypeptide or polynucleotide can be custom ordered from commercial sources.

The term “binding” as used herein includes a non-covalent interaction between macromolecules (e.g., between a protein and a polynucleotide, between a polynucleotide and a polynucleotide, and between a protein and a protein). Such non-covalent interaction is also referred to as “associating” or “interacting” (e.g., when a first macromolecule interacts with a second macromolecule, the first macromolecule binds to second macromolecule in a non-covalent manner). Some portions of a binding interaction may be sequence-specific; however, all components of a binding interaction do not need to be sequence-specific, such as a protein's contacts with phosphate residues in a DNA backbone. Binding interactions can be characterized by a dissociation constant (Kd). “Affinity” refers to the strength of binding. An increased binding affinity is correlated with a lower Kd. An example of non-covalent binding is hydrogen bond formation between base pairs.

As used herein, the term “isolated” can refer to a nucleic acid or polypeptide that, by the hand of a human, exists apart from its native environment and is therefore not a product of nature. Isolated means substantially pure. An isolated nucleic acid or polypeptide can exist in a purified form and/or can exist in a non-native environment such as, for example, in a recombinant cell.

As used herein, a “host cell” generally refers to a biological cell. A cell can be the basic structural, functional and/or biological unit of a living organism. A cell can originate from any organism having one or more cells. Examples of host cells include, but are not limited to: a prokaryotic cell such as a bacterial cell, a eukaryotic cell, an archaeal cell, a cell of a single cell eukaryotic organism, a protozoa cell, a cell from a plant, an algal cell, seaweeds, a fungal cell, an animal cell, a cell from an invertebrate animal, a cell from a vertebrate animal, or a cell from a mammal. Furthermore, a cell can be a stem cell or progenitor cell.

The present invention is directed to compositions and methods for making genomic changes in prokaryotes using recombination mechanisms, such as homologous recombination. In particular, single plasmid systems for genome editing using targeted genome editing and selection strategies, such as by utilizing programmable CRISPR proteins and, in some cases, anti-CRISPR proteins and peptides, are described herein. The single plasmid systems described herein provide the ability to genetically engineer bacterial strains that are difficult to transform. The systems used herein also increase the efficiency of genomic engineering in tractable strains. In some embodiments, the plasmid designs described herein allow for transformation of more than one bacterial species. Additionally, the present methods allow for selection of mutated genomes without the requirement of incorporating antibiotic resistance into the targeted genome.

Common methods for making genomic changes to the bacterial chromosome include the insertion of an antibiotic resistance gene into the host cell genome so that the engineered cells can be selected by growth in culture that includes a specific antibiotic. Methods that make use of lambda RED recombineering enzymes also require that the genome be open at the site of homology in order for homologous DNA to be inserted into the genome at the specific targeted site. Cells that have been engineered will survive antibiotic exposure, but those that have not been engineered will not survive such exposure. However, many downstream applications of genomic engineering require the removal of the antibiotic resistance gene. Several strategies exist for removing the antibiotic resistance gene, but these often leave small changes to the genome known as “scars.” The requirement for antibiotic gene removal in serial genomic manipulations significantly adds to the time required to generate an engineered strain and leaves multiple scars in the genome that may cause genomic instability.

By utilizing a programmable endonuclease, the incorporation of an antibiotic resistance gene into the host cell genome can be avoided. Plasmids described herein can contain an antibiotic resistance gene or genes in order to identify cells that have received the plasmid so that these cells can be selected and cells that have not received the plasmid can be excluded. However, unlike other gene editing procedures, the antibiotic resistance gene or genes are not transferred from the plasmid into the host cell genome. Therefore, as cells grow without antibiotic selection, some cells will lose the plasmid. This process is known as “plasmid curing.” The size of the plasmid and the metabolic cost on the cell impact the rate of plasmid curing. Once the plasmid has been cured, the cells will no longer be resistant to any antibiotics. The rate of plasmid curing can be increased through the use of CRISPR enzymes. Cells can be monitored and determined to have cured the plasmid and thus antibiotic resistance through a number of assays including, without limitation, PCR, qPCR, ddPCR, Sanger sequencing, next generation sequencing, plating, growth assays, and the like.

In one embodiment, a programmable Cas endonuclease is used, such as Cas9. In bacteria, when both Cas9 and a guide RNA (gRNA) are present, they will form a complex that targets the bacterial chromosome and Cas9 will make a DSB in the bacterial chromosome at the targeted site. In order to survive, the bacteria must repair the DSB before replicating their genome. If the DSB cannot be repaired before the DNA polymerase (DNAP) reaches the break, the cell will die. Most bacteria do not have a NHEJ repair pathway that would be used to simply rejoin the DSB, but instead only have a homologous recombination (HR) repair pathway which requires a strand of homologous DNA in order to repair the break.

By providing a template for HR that includes changes to the bacterial genome, the native recombination pathways in the bacterium can perform HR. The possibility of re-cutting by a Cas9 endonuclease is removed by designing the HR template so that the Cas9 endonuclease recognition site is destroyed after successful HR. Cells that undergo the desired genomic edit will not be impacted further by the Cas9 endonuclease, but those that do not will be killed. Therefore, Cas9 can be used as a selection strategy for making changes to the bacterial genome and the requirement for incorporating antibiotic resistance into the host cell genome can be avoided. In some embodiments, an anti-CRISPR peptide or protein is co-expressed with Cas9 in the bacteria. The anti-CRISPR renders prematurely expressed and overexpressed Cas9 inactive, thereby increasing the efficiency of HR-positive transformants. Unlike previous methods that use individual plasmids to deliver the components for genome editing, the present system uses a single plasmid encoding the various components under the control of individual promoters.

In certain embodiments, the single plasmids for use in the present methods include sequences encoding a programmable protein; a guide polynucleotide for targeted genomic DNA cleavage; optionally a donor polynucleotide with homology arms (if an insertion into the genome is desired); optionally homology arms without a donor polynucleotide (if a targeted deletion of a sequence in the genome is desired); optionally an anti-CRISPR peptide; and control elements that regulate expression of the various components.

In some instances, sequences coding for the programmable protein are under the control of an inducible promoter. Many inducible promoters are known in the art and will find use in driving expression of the programmable endonuclease. Such promoters include those induced by growth in particular sugars, such as L-arabinose, L-rhamnose, xylose, lactose and sucrose; promoters induced by antibiotics, such as tetracyclines; promoters induced by other chemical compounds such as substituted benzenes, cyclohexanone-related compounds, ε-caprolactam, propionate, thiostrepton, alkanes, and peptides. For a review of inducible promoters see, e.g., Brautaset, et al., Microb Biotechnol. (2009) 2:15-30.

For example, inducible promoters for use in the plasmid systems described herein include those derived from bacterial operons. A bacterial transcriptional operator is a sequence of DNA adjacent to a promoter that serves as a binding site for transcriptional activators and repressors. Activators recruit RNA polymerase (RNAP) to a promoter leading to transcription of the gene associated therewith. Repressors block RNAP binding to a promoter leading to inhibition of gene transcription.

Non-limiting particular examples of promoters that will find use in driving expression of the programmable protein portion of the plasmid, include promoters derived from, for example, the tet, lac, ara, lambda, arginine operon transcription control sequences. These promoters are activated when the transformed organism is grown in the presence of their corresponding inducing molecule. For example, tetracyclines and analogs thereof, such as anhydrotetracycline (aTc) activate tet-inducible promoters; lactose molecules such as Isopropyl β-D-1-thiogalactopyranoside (IPTG) and allolactose activate lac-inducible promoters; and arabinose activates ara-inducible promoters. Many such promoters derived from these and other operons are known.

In one embodiment of the invention, the gene encoding a programmable protein, such as a Cas9, is operably linked to a tet promoter, such as a tetO promoter, e.g., a tetO₂ promoter. If a tet promoter is present, the plasmid will also include a tet transcriptional regulator, TetR, that will bind to the operator in the absence of tetracycline and inhibit expression of the programmable endonuclease. In the presence of tetracycline or tetracycline analogs such as, but not limited to aTc and doxycycline, TetR no longer binds to the tet operator, which relieves the repression on the gene encoding the programmable nuclease, allowing it to be transcribed. Although the present invention is illustrated using an inducible tet promoter, other bacterial promoters can also be used in the present invention.

The coding sequence for the programmable endonuclease that is operably linked to the promoter codes for a protein that can be guided to target nucleotide sequences and is capable of introducing double-strand breaks or nicks within these sequences, or is capable of binding tightly to the target nucleotide sequences thus blocking transcription of a particular gene or genes. Programmable endonucleases for use in the present methods include, without limitation, those from the CRISPR-Cas systems, as described herein, ZFNs, TALENs, meganucleases, MegaTALs, Argonaute (Ago), and others known to one of skill in the art. See, e.g., Gao, et al., Nature Biotechnology (2016) 34:768-773.

In some embodiments, the programmable endonuclease is a Cas9 protein. A number of catalytically active Cas9 proteins are known in the art and a Cas9 protein for use herein can be derived from any bacterial species, subspecies or strain that encodes the same. Although in certain embodiments herein, the methods are exemplified using S. pyogenes Cas9 (SpyCas9), orthologs from other bacterial species will find use herein. The specificity of these Cas9 orthologs is well known. Also useful are proteins encoded by Cas9-like synthetic proteins, and variants and modifications thereof. The sequences for hundreds of Cas9 proteins are known and any of these proteins will find use with the present methods.

Additionally, it is to be understood that other Cas nucleases, in place of or in addition to Cas9, may be used, including any of the Cas proteins from any of the various CRISPR-Cas classes, types, and subtypes.

Alternatively, programmable protein molecules can be used that retain site-directed binding capability but lack the ability to make DSBs in the target sequence. For example, the plasmid can be designed to incorporate a sequence encoding a Cas nickase that maintains the ability to bind to and make a single-strand break at a target site. For Cas9, such a mutant (termed “nCas9” herein) will typically include a mutation in one, but not both of the Cas9 endonuclease domains (HNH and RuvC). Thus, an amino acid mutation at position D10A or H840A in Cas9, numbered relative to S. pyogenes, can result in the inactivation of the nuclease catalytic activity and convert Cas9 to a nickase enzyme that makes single-strand breaks at the target site. In this embodiment, when expressed in a cell with a guide polynucleotide, such as sgRNA designed to target the bacterial genome, the cell should not die, but one of several DNA repair pathways will be activated, resulting in opening the genome at the site of the ssDNA break, thus enhancing genome editing efficiency. Additionally, the nCas9 can be used with more than one guide to target several target sites in the genome. Target sites can be close together (e.g., 20 bp or less apart) or farther apart (e.g., 1000-2000 bp or more apart).

Additionally, a programmable protein can be used that has been mutated such that it is incapable of making any breaks in the target genome, but that still binds tightly to the targeted region of the genome when directed by a guide polynucleotide. For example, a dCas9 can be used that includes mutations that inactivate Cas9 nuclease function. This is typically accomplished by mutating both of the two catalytic residues (D10A in the RuvC-1 domain, and H840A in the HNH domain, numbered relative to S. pyogenes Cas9) of the gene encoding Cas9. The Cas9 double mutant with changes at amino acid positions D10A and H840A lacks both the nuclease and nickase activities, but still retains the ability to tightly bind DNA targeted by the guide polynucleotide. This blocks RNA polymerase from accessing the genome. By preventing RNAP from accessing the genome, mRNA transcription cannot occur, and therefore protein translation cannot occur. Thus, expressing dCas9 with a guide polynucleotide that targets the genome serves as a way to turn off specific genes in the bacterial genome.

Plasmids for use herein will also include sequences for one or more guide polynucleotides. The guide(s) is designed to target particular regions of a gene present in the target bacterial strain transformed with the plasmid, and when complexed with the programmable endonuclease, guides the endonuclease to the host cell target sequence for cleavage. Multiple guide polynucleotides can be used in order to target multiple sites in a host cell genome. Representative complexes are those between a Cas protein, such as a Cas9 protein, with a sgRNA (sgRNA/Cas9 complex). The complex can be directed precisely toward sites of interest within the host cell genome. The guide polynucleotides, e.g., sgRNAs, can be designed to target any DNA sequence containing the appropriate PAM necessary for each Cas protein, such as Cas9 or Cas12a. Additional PAMs can also be created in the target DNA using a type of codon optimization, where silent mutations are introduced into amino acid codons in order to create new PAM sequences. For example, for strategies using Cas9, which recognizes an NGG PAM, a CGA serine codon can be changed to CGG, preserving the amino acid coding but adding a site where double-strand breaks can be introduced. Moreover, computational analysis on small insertions shows that Cs and Gs are inserted with high frequency. This can be used to create new PAMs. The entire coding region or parts of the coding region can thus be optimized with suitable PAM sites on the coding and non-coding strand. PAM optimized DNA sequences can be manufactured and cloned into suitable vectors for transformation into the ultimate host cell.

Although in some embodiments described herein sgRNA is used as an exemplary guide polynucleotide, it will be recognized by one of ordinary skill in the art that other guide polynucleotides that site-specifically guide endonucleases, such as CRISPR-Cas proteins to a target nucleic acid, can be used.

The plasmids for use herein will also optionally include a sequence for a donor polynucleotide (donor PN) that includes a genome editing sequence that imparts a genomic change to a target site present in a host cell genome, e.g., an insertion or a deletion. The genome editing sequence is flanked by homology arms, also present in the plasmids, in order to site-specifically incorporate the genomic change into a target site present in the host cell genome. The target site in the genome, and the genome editing sequence, can be chosen such that an endogenous gene is rendered inoperative or is partially or fully removed. The genome editing sequence can comprise one or more gene sequences and one or more operably linked promoters, and one or more gene sequences operably linked to an endogenous promoter.

In certain embodiments where non-specific genomic deletions are desired, plasmids for use in the invention can be constructed that lack a donor PN and associated homology arms. In this embodiment, by including sequences for a guide molecule and a programmable endonuclease, such as but not limited to, a sgRNA and a Cas9, a specific sequence of the genome will be cleaved, but the cell is not provided with a repair template that instructs the cell to repair the DSB caused by Cas9. This results in either cell death from the DSB, or the rearrangement of the host cell genome through recombination. This leaves a large, variable, and non-specific deletion in the genome, which can remove the genomic sequence where Cas9 binds. As used herein, the term “high recombination ability” refers to organisms that can rearrange their genomes when provided with the plasmid elements described herein, without a donor PN and homology arms.

In additional embodiments where the plasmids lack a donor PN and associated homology arms, a catalytically inactive programmable protein, such as a dCas9, can be used to tightly bind the host cell genome at a site prescribed by the guide molecule, such as sgRNA, without generating any DNA breaks; and cell death will not occur. By binding the genome at specific sites, transcription of a specific gene, or group of genes, can be accomplished without permanently altering the genome.

The plasmids described herein also can include a coding sequence for an anti-CRISPR (Acr) molecule, i.e., a molecule that inhibits the function of CRISPR-Cas systems. Several Acrs are known and are typically found in phages, genomic islands, and prophages. See, e.g., Bondy-Denomy, et al., Nature (2013) 493:429-432 (2013); Rauch, et al., Cell (2017) 168:150-158; Pawluk, et al., mBio (2014) 5:e00896-14; Pawluk, et al., Nat. Microbiol. (2016) 1:16085; Pawluk, et al., Cell (2016) 167:829-1838; Hynes, et al., Nature Microbiology (2017) 2:1374-1380. Most of these molecules are small proteins, approximately 50-150 amino acids in length.

In some cases, the Acrs possess a target sequence with identity to a CRISPR spacer in the host cell. In order to perform genomic engineering using CRISPR enzymes, cells are engineered to contain a spacer with a perfect match to their own genomic DNA. This is called a “self-targeting” guide. Bacteria that contain self-targeting guides require precise control of CRISPR-Cas inactivation. This can be achieved through regulation of transcription through promoters and inhibitors of RNA polymerase, or by regulation of protein activity through Acrs that inhibit enzyme activity. In the absence of precise control of CRISPR-Cas activation, the host genome will be cleaved resulting in unwanted cell death. Stochastic expression of genes on plasmids has been observed throughout microbiology. Normally, some amount of stochastic expression can be tolerated by the cell. However, expressing even one copy of a Cas9 endonuclease and a self-targeting guide can lead to bacterial cell death. Thus, in order to prevent Cas9-mediated death, plasmids for use herein can contain a gene encoding an Acr molecule under the control of a constitutive promoter. A constitutive promoter allows for continuous transcription of its cognate gene. Hundreds of constitutive promoters for use in prokaryotes are known in the art and include, without limitation, any of the BBa promoters listed in the Registry of Standard Biological Parts (parts.igem.org/Promoters/Catalog/Constitutive), such as, but not limited to, any of BBa_J23100 to BBa_J23119. The choice of promoter will depend on the microorganism transformed by the plasmid in question, e.g., a promoter recognized by the RNAP present in the particular prokaryote used.

In some cases, the promoter is a constitutive promoter with low activity relative to the activity of the promoter driving expression of the programmable endonuclease. Typically, the expression of a library of promoters is scored relative to the highest expressing promoter in a specific context. Thus, in the present case, mRNA produced from each promoter can be measured, and if, for example, the amount of Cas9 mRNA produced is considered 100%, the promoter driving acr expression is considered a “low activity” constitutive promoter if an amount of Acr mRNA produced is less than 25%, such as less than 20% . . . 15% . . . 10% . . . 5%, or even lower, while still being active. Similarly, a constitutive promoter is considered to have “high activity” if the amount of mRNA produced relative to the reference promoter is above 50% . . . 60% . . . 70% . . . 75% . . . 80% . . . 85% . . . 90%, etc. The design and construction of variable-strength constitutive promoters is known and described in, e.g., Davis, et al., Nucl. Acids Res. (2010) 39:1131-1141.

The Acr molecule used is typically one with high affinity for the particular programmable endonuclease used, such as Cas9. Even one copy of the Acr can completely block the activity of one Cas9 enzyme. The promoter controlling expression of the Acr can be chosen so that a low amount of the Acr will exist in the cell at all times. If there is stochastic production of Cas9 endonuclease, the Acr will inhibit Cas9 and prevent Cas9-mediated cell death. However, when the cas9 gene is activated, more Cas9 endonuclease will be produced than can be inhibited by the Acr molecule, and Cas9 will be able to cleave unengineered DNA.

Many Acr molecules are known, the choice of which will depend on the particular programmable endonuclease used. For example, several proteins derived from phages block the function of Class 1 CRISPR-Cas systems. At least ten subtype I-F acr genes and four subtype I-E acr genes are known (see, e.g., Pawluk, et al., Nat. Microbiol. (2016) 1:16085). Additionally, several Acr proteins inhibit Class 2 CRISPR-Cas9 systems such as, but not limited to, Acrs from prophages of Listeria monocytogenes, including, without limitation, AcrIIA1, AcrIIA1-2, AcrIIA2 and AcrIIA4. In addition to L. monocytogenes Cas9, AcrIIA2 and AcrIIA4 also inhibit SpyCas9 (see, e.g., Rauch, et al., Cell (2017) 168:150-158). Similarly, AcrIIA5 from a virulent phage of S. thermophiles also inhibits SpyCas9 (Hynes, et al., Nature Microbiology (2017) 2:1374-1380). Additional Acrs that target particular programmable endonucleases can be readily identified using techniques known in the art, such as those described in, for example, Rauch, et al., Cell (2017) 168:150-158.

The plasmids described herein can also contain sequences coding for a selectable marker such that the plasmids can be detected and isolated from a propagation strain (discussed further below). Using the plasmids of the invention, the antibiotic resistance gene or genes are not transferred from the plasmid into the host cell genome and therefore downstream removal of these genes is not required. More than one selectable marker gene can be used in the plasmids described herein, such as where selection in two different bacterial strains is desired.

Selectable markers are well known in the art and include genes that render bacteria resistant to drugs such as ampicillin, chloramphenicol, erythromycin, kanamycin, neomycin, gentamicin, tetracycline, and the like. Selectable markers can also include biosynthetic genes, such as those in the histidine, tryptophan, and leucine biosynthetic pathways.

In embodiments where the selected host cell lacks homologous recombination activity, a sequence encoding a heterologous recombinase enzyme compatible with the host can be added to the plasmids. Such recombinase enzymes are known and include, for example, bacteriophage-derived recombinase operons, such as those described in, e.g., Guo, et al., Microb. Cell Fact. (2019) 18:22 (for Lactococcus lactis); Xin, et al., FEMS Microbiol. Lett. (2017) 364:fnx243 (for Lactobacillus casei); Yang, et al., Microb Cell Fact. (2015) 14:154 (for Lactobacillus plantarum); Zhang, et al., Nat Genet. (1998) 20:123-128; Yu, et al., Proc. Natl. Acad. Sci. USA (2000) 97:5978-5983 (for E. coli); van Kessel, et al., Nat. Methods (2007) 4:147-152 (for Mycobacterium tuberculosis); Yin, et al., Nucleic Acids Res. (2015) 43:e36 (for Photorhabdus and Xenorhabdus); lambda RED recombineering enzymes; Cre recombinase; Hin recombinase; Tre recombinase; flp recombinase (see, e.g., Nafissi, et al., Appl. Microbiol. Biotech. (2014 98:2841-2851; Menouni et al., FEMS Microbiol. Letters (2015) 362:1-10 for reviews of bacteriophage-derived recombinases).

In addition to those elements described above, the plasmids can also contain sequences encoding degradation tags for promoting degradation of the programmable endonuclease. Such tags are short peptide sequences that mark a protein for degradation by the cell's protein recycling machinery. In doing so, the degradation tag effectively decreases the protein half-life or the typical length of time that a protein will exist in the cell, once translated. An example of a representative degradation tag that functions in E. coli is ssrA.

Plasmids for use in the present invention, with or without one or more of the above elements, are constructed using methods well known in the art, such as, but not limited to sequence- and ligation-independent cloning (SLIC). SLIC uses an exonuclease, such as a T4 DNA polymerase, to generate single-stranded DNA overhangs in insert and vector sequences. See, e.g., Li, et al., Meth. Mol. Biol. (2012) 852:51-59; Jeong, et al., Appl. Environ. Microbiol. (2012) 78:5440-5443. Other methods, such as, but not limited to, Gibson Assembly, Golden Gate Assembly, site-directed mutagenesis, restriction enzyme digestion and ligation, and the like, can also be used in order to construct the plasmids described herein.

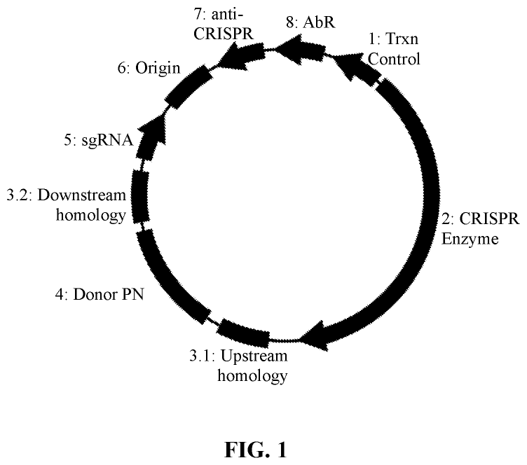

Representative plasmid element organizations are shown in FIGS. 1-10 (termed “Plasmid Element Organizations A-J,” respectively).

FIG. 1 depicts a generic structure for a plasmid having Plasmid Element Organization A. The elements and their organization in the plasmid are shown. Element 1 is a transcriptional control unit, termed “Trxn Control” in FIG. 1, and includes an inducible promoter as described herein. Element 2 codes for is a CRISPR enzyme, such as a CRISPR-Cas protein. Transcription of the CRISPR-Cas gene, such as a cas9, can be under the control of an inducible promoter. Element 4 is a donor PN and Elements 3.1 and 3.2 are upstream and downstream homology arms, respectively, so that all or a portion of the donor PN can be inserted into the host cell genome via homologous recombination at the target site. Element 5 is a guide, such as sgRNA, that complexes with the CRISPR enzyme upon expression. Element 6 is an origin of replication that enables replication of the plasmid and segregation of replicated plasmids into daughter cells. A number of bacterial origins of replication are known and will find use herein. Non-limiting examples include ColE1, pMB1 and derivatives thereof, pSC101, R6K, and 15A. Element 7 codes for an anti-CRISPR molecule as described herein. The gene for the anti-CRISPR can be regulated by a weak constitutive promoter, to result in a constant, low-level expression of the anti-CRISPR. Element 8 is an antibiotic resistance gene (“AbR”), as described herein. Although FIG. 1 includes Elements 3.1, 3.2 and 4, in some cases, one or more of these elements can be excluded from this plasmid organization, to provide for a targeted deletion rather than an insertion.

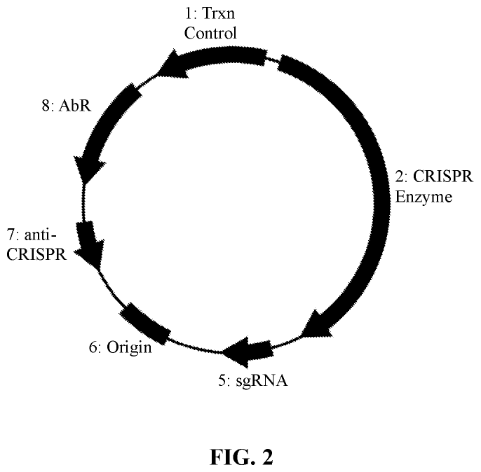

FIG. 2 depicts a generic structure for a plasmid having Plasmid Element Organization B. This plasmid includes Elements, 1, 2, 5, 6, 7, and 8, as described above, but lacks Elements 3.1, 3.2, and 4 (the donor PN and homology arms), and can be used to make deletions in the host cell genome.

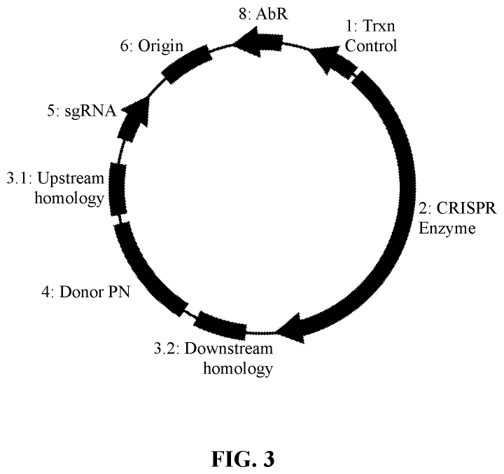

FIG. 3 shows a generic structure for a plasmid having Plasmid Element Organization C. This plasmid includes Elements 1, 2, 3.1, 3.2, 4, 5, 6, and 8, as described above, but lacks Element 7 (the anti-CRISPR). Although FIG. 3 includes Elements 3.1, 3.2 and 4, in some cases, Element 4, as well as Elements 3.1 and 3.2, if desired, can be excluded from this plasmid organization when a deletion, rather than insertion, is desired.

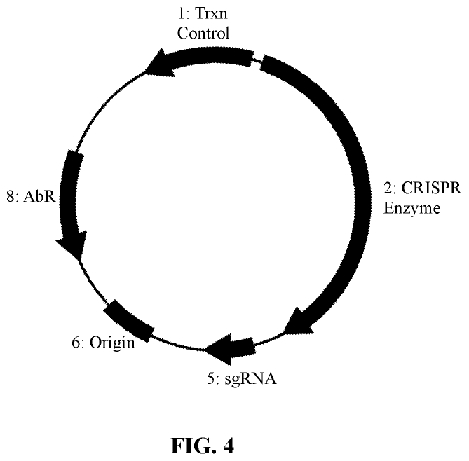

FIG. 4 shows a generic structure for a plasmid having Plasmid Element Organization D. This plasmid includes Elements 1, 2, 5, 6, and 8, as described above, but lacks Elements 3.1, 3.2, 4, and 7 (a gene encoding an anti-CRISPR molecule) and can be used to make deletions in the host cell genome.

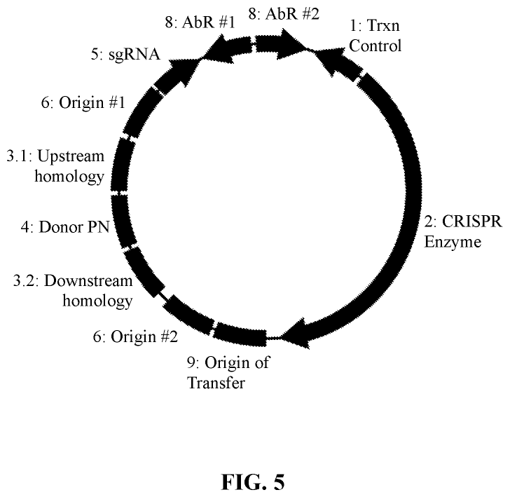

FIG. 5 shows a generic structure for a plasmid having Plasmid Element Organization E. This plasmid includes Elements 1, 2, 3.1, 3.2, 4, 5, 6 (two origins are present, allowing for replication in two different bacteria) and 8 (two antibiotic resistance genes are present, allowing for selection in two different bacteria), as well as Element 9 which is an origin of transfer. Plasmids with this structure can therefore be used in conjugation reactions.

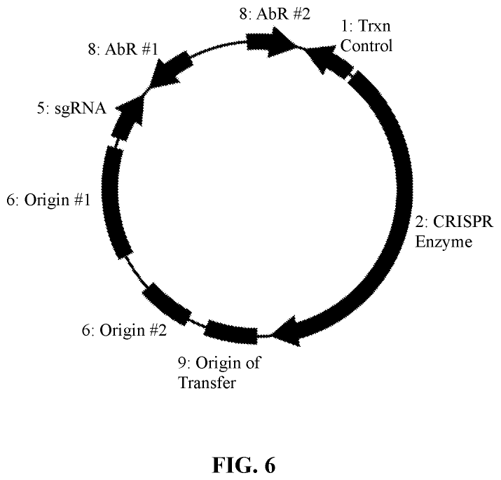

FIG. 6 shows the generic structure for a plasmid having Plasmid Element Organization F. This plasmid includes Elements 1, 2, 5, 6 (two origins are present, allowing for replication in two different bacteria) and 8 (two antibiotic resistance genes are present, allowing for selection in two different bacteria), as well as Element 9. Plasmids with this structure can also be used in conjugation reactions. Plasmid Element Organization F lacks Elements 3.1, 3.2, 4, and 7 and can be used to make deletions in the host cell genome.

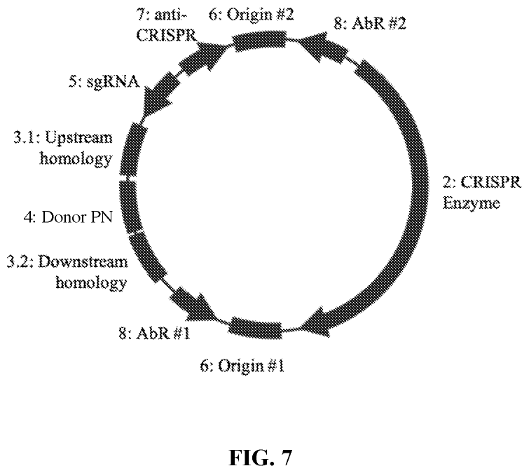

FIG. 7 shows the generic structure for a plasmid having Plasmid Element Organization G. This plasmid includes Elements 2, 5, 6 (two origins are present, allowing for replication in two different bacteria), 7, and 8 (two antibiotic resistance genes are present, allowing for selection in two different bacteria). Elements 3.1, 3.2 and 4 are absent. Plasmids with this structure can be used to make deletions in the host cell genome. In some embodiments, Plasmid Element Organization G can include a donor PN with associated homology arms in order to make insertions into a host cell genome.

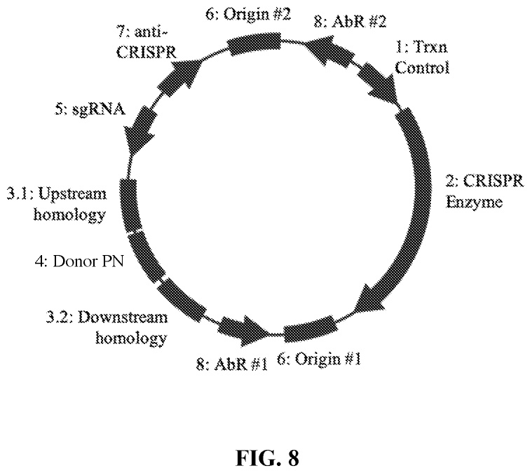

FIG. 8 shows the generic structure for a plasmid having Plasmid Element Organization H. This plasmid includes Elements 1, 2, 3.1, 3.2, 4, 5, 6 (two origins are present, allowing for replication in two different bacteria), 7, and 8 (two antibiotic resistance genes are present, allowing for selection in two different bacteria).

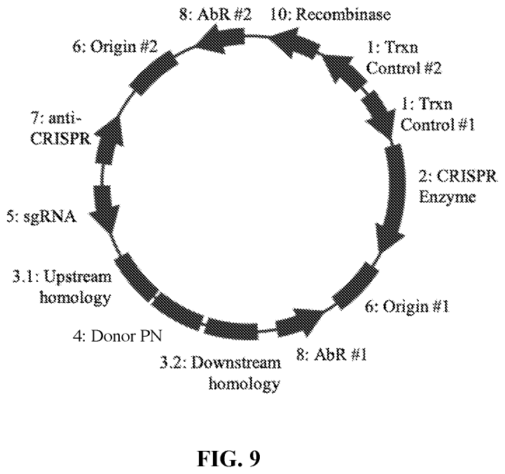

FIG. 9 shows the generic structure for a plasmid having Plasmid Element Organization I. This plasmid includes Elements 1, 2, 3.1, 3.2, 4, 5, 6 (two origins are present, allowing for replication in two different bacteria), 7, and 8 (two antibiotic resistance genes are present, allowing for selection in two different bacteria). Element 10, encoding a heterologous recombinase, is present so that bacteria lacking endogenous recombination capacity, e.g., Lactobacillus, can perform homologous recombination. Two transcriptional control units (Element 1) are present, one to regulate Element 2 and one to regulate Element 10. These control units can include inducible promoters.

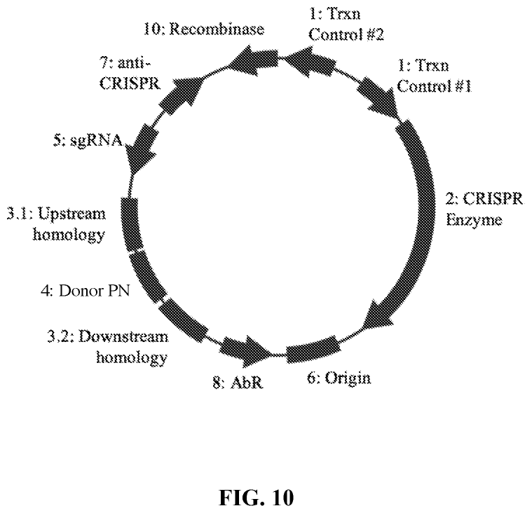

FIG. 10 shows the generic structure for a plasmid having Plasmid Element Organization J. This plasmid includes Elements 1 (two transcriptional control units are present to regulate Element 2 and Element 10), 2, 3.1, 3.2, 4, 5, 6, 7, 8, and 10. As explained above, Element 10 allows bacteria lacking endogenous recombination capacity to perform homologous recombination.

As is apparent, any of the plasmids described herein can include more than one guide polynucleotide, more than one origin, more than one antibiotic resistance gene, more than one donor PN, more than one transcriptional control unit, etc.

Table 1 details particular representative plasmids for use in gene editing. Representative polynucleotide sequences that can be included in these plasmids are shown in Table 2. These plasmids are described in detail in the Examples. It is to be understood that the various components of the plasmids detailed in Table 1 are representative and the invention is not limited to the plasmids described in Table 1 or the sequences in Table 2.

TABLE 1
Representative Plasmid Structures

| Plasmid | Element | |
|---|---|---|
| No. | Organization | Element Description |
| Plasmid 1 | A (FIG. 1) | Element 1: tetR (SEQ ID NO: 1); inducible promoter tet |
| | | promoter (SEQ ID NO: 2); RBS (SEQ ID NO: 3). Element 2: |
| | | cas9 (SEQ ID NO: 4). Elements 3.1 and 3.2 (SEQ ID NO: 5 and |
| | | SEQ ID NO: 6, respectively): homologous to regions on either |
| | | side of E. coli tonA, respectively. Element 4: Donor PN |
| | | comprising SEQ ID NOS: 7 and 8. Element 5: Single sgRNA |
| | | unit (SEQ ID NO: 9) targeting Cas9 to the tonA region (SEQ ID |
| | | NO: 9). Element 6: Origin p15A (SEQ ID NO: 10). Element 7: |
| | | anti-CRISPR acrIIA4 (SEQ ID NO: 11), weak constitutive |
| | | promoter (SEQ ID NO; 13), RBS (SEQ ID NO: 12). Element 8: |
| | | chloramphenicol resistance gene (SEQ ID NO: 14). |
| Plasmid 2 | B (FIG. 2) | Elements 1, 2, 5, 6, 7 and 8 of Plasmid 1. Elements 3.1, 3.2, and |
| | | 4 of Plasmid 1 absent. |
| Plasmid 3 | B (FIG. 2) | Elements 1, 5, 6, 7 and 8 of Plasmid 1. Element 2: cas9 (SEQ |
| | | ID: 19) differed from Plasmid 1 and 2. Elements 3.1, 3.2, and 4 |
| | | absent. |
| Plasmid 4 | A (FIG. 1) | Elements 1, 3.1, 3.2, 4, 6, 7 and 8 of Plasmid 1. Element 2: |
| | | nCas9 (SEQ ID NO: 20). Element 5: two tandem sgRNA units |
| | | selected from sgRNA unit pair #1 (SEQ ID NO: 21); sgRNA |
| | | unit pair #2 (SEQ ID NO: 22); and sgRNA unit pair #3 (SEQ ID |
| | | NO: 23). |
| Plasmid 5 | C (FIG. 3) | Elements 1, 3.1, 3.2, 4, 6, and 8 of Plasmid 1. Element 2: nCas9 |
| | | (SEQ ID NO: 20). Element 5: two tandem sgRNA units selected |
| | | from sgRNA unit pair #1 (SEQ ID NO: 21); sgRNA unit pair #2 |
| | | (SEQ ID NO: 22); and sgRNA unit pair #3 (SEQ ID NO: 23). |
| | | Element 7 (anti-CRISPR) was deleted. |
| Plasmid 6 | B (FIG. 2) | Elements 1, 6, 7 and 8 of Plasmid 1. Elements 3.1, 3.2, and 4 of |
| | | Plasmid 1 absent. Element 2: dcas9 (SEQ ID NO: 26); Element |
| | | 5: four sgRNA units (SEQ ID NO: 27). |
| Plasmid 7 | D (FIG. 4) | Elements 1, 6, and 8 of Plasmid 1. Element 2: dcas9 (SEQ |
| | | ID: 26) Elements 3.1, 3.2, and 4 of Plasmid 1 absent. Element 5: |
| | | four sgRNA units (SEQ ID NO: 27). |
| Plasmid 8 | B (FIG. 2) | Elements 1, 5, 6, and 8 of Plasmid 1. Element 2: cas9 (SEQ |
| | | ID: 19). Elements 3.1, 3.2, and 4 of Plasmid 1 absent. Element |
| | | 7: anti-CRISPR (SEQ ID NO: 41). |
| Plasmid 9 | E (FIG. 5) | Element 1: tetR, a RBS, a tet promoter, and a cas9 promoter |
| | | (SEQ ID NOS: 28-31, respectively). Element 2: cas9 (SEQ ID |
| | | NO: 32). Elements 3.1 and 3.2 (SEQ ID NO: 35 and SEQ ID |
| | | NO: 36, respectively) homologous to regions on either side of B. |
| | | thetaiotaomacron tdk. Element 4: Donor PN (can be absent); |
| | | Element 5: Single sgRNA unit (SEQ ID NO: 38) targeting Cas9 |
| | | to the tdk region; (SEQ ID NO: 9). Element 6: Origin #1 (SEQ |
| | | ID NO: 37) for replication in Bacteroides and Origin #2 (SEQ |
| | | ID NO: 34) for replication in E. coli. Element 8: AbR #1 (SEQ |
| | | ID NO: 39) for selection in Bacteroides and AbR #2 (SEQ ID |
| | | NO: 40) for selection in E. coli. Element 9: Origin of Transfer |
| | | (SEQ ID NO: 33). Element 7 (anti-CRISPR) absent. |
| Plasmid 10 | F (FIG. 6) | Elements 1, 2, 5, 6, 8, and 9 of Plasmid 9. Elements 3.1, 3.2, |
| | | and 4 of Plasmid 9 absent. Element 7 (anti-CRISPR) absent. |
| Plasmid 11 | G (FIG. 7) | Element 2: cas9 (SEQ ID NO: 44). Elements 3.1 and 3.2 (SEQ |
| | | ID NO: 46 and SEQ ID NO: 47, respectively) homologous to |
| | | regions on either side of Lactobacillus paracasei gene |
| | | LSEI_2368 (SEQ ID NO: 45). Element 5: Single sgRNA unit |
| | | (SEQ ID NO: 48) targeting Cas9 to the LSEI_2368 region of the |
| | | L. paracasei genome. Element 6: Origin #1 (SEQ ID NO: 49) |
| | | for replication in Lactobacillus, and Origin #2 (SEQ ID NO: 50) |
| | | for replication in E. coli. Element 7: anti-CRISPR acrIIA4 |
| | | (SEQ ID NO: 51). Element 8: AbR #1 (SEQ ID NO: 52) for |
| | | selection in Lactobacillus and AbR #2 (SEQ ID NO: 53) for |
| | | selection in E. coli. Element 1 (Trxn Control) and Element 4 |
| | | (Donor PN) absent. |
| Plasmid 12 | H (FIG. 8) | Element 1 (Trxn Control). Elements 2, 3.1, 3.2, 5, 6, 7, and 8 of |
| | | Plasmid 11. |
| Plasmid 13 | I (FIG. 9) | Element 1: Trxn Control #1 (spp-derived_inducible promoter |
| | | system, SEQ ID NO: 54), and Trxn Control #2 (nis-derived |
| | | inducible promoter system, SEQ ID NO: 57). Elements 2, 3.1, |
| | | 3.2, 5, 6, 7, and 8 of Plasmid 12. Element 10: encodes a |
| | | heterologous recombinase operon (SEQ ID NO: 59). |
| Plasmid 14 | J (FIG. 10) | Elements 1, 2, 3.1, 3.2, 5, 6, 7, 8 and 10 of Plasmid 13. Origin |
| | | #2 and AbR #2 from Plasmid 13 absent. |

In order to generate large quantities of the plasmids for genomic engineering, a plasmid is transformed into a propagation strain. Methods of introducing plasmids into host cells are known in the art and are typically selected based on the host cell used. Such methods include, for example, viral or bacteriophage transduction, transfection, conjugation, electroporation, chemical transformation, calcium phosphate precipitation, polyethyleneimine-mediated transfection, DEAE-dextran mediated transfection, protoplast fusion, lipofection, liposome-mediated transfection, particle gun technology, direct microinjection, and nanoparticle-mediated delivery. Such techniques are described in, for example, Methods in Molecular Biology (Series), J. M. Walker, ISSN 1064-3745, Humana Press; Methods in Enzymology (Series), Academic Press; Molecular Cloning: A Laboratory Manual (Fourth Edition), 2012, M. R. Green, et al., Cold Spring Harbor Laboratory Press. See also Sternberg, et al., Meth. Enzymol. (1991) 204:18-43; Kwoh, et al., J. Virol. (1978) 27:535-550 for methods of viral/bacteriophage transduction.

If the transcriptional repressor (e.g., TetR) that inhibits transcription of the programmable endonuclease is not present in the plasmid described herein, the propagation strain is designed to express a transcriptional repressor that will inhibit transcription of the programmable endonuclease. For example, if the tet promoter is used and the plasmid lacks the tetR gene, the propagation strain must express enough of the tetR gene so that the transcription factor TetR is present at high enough concentrations to bind to the tet operator on the plasmid and inhibit transcription. Therefore, to make this propagation strain, the tetR gene is added to the bacterial genome under the control of a high activity constitutive promoter as described herein. The tetR gene can be placed anywhere in the genome that will not disrupt the ability of the bacterium to grow under conditions that produce large quantities of the plasmid. One non-limiting example is to replace the lacZ gene with the tetR gene. This can be accomplished using techniques well known in the art. See, e.g., Reisch, et al., Scientific Reports (2015) 5:15096; Court, et al., Annual Review of Genetics (2002) 36:361-388. Additionally, the propagation strain can be cultured in a medium that includes components for selection, such as an appropriate antibiotic if an antibiotic resistance gene has been engineered into the plasmid of the invention.

Once the plasmid is sufficiently propagated in the propagation strain, it is isolated and transformed into a target bacterial strain, using methods well known in the art and described herein. The target bacterial strain lacks the repressor molecule that represses expression of the programmable nuclease. Representative target strains for use in the subject invention, include, without limitation, bacterial hosts such as gram-negative or gram-positive bacteria, including e.g., bacteria from the phylum Proteobacteria, including, but not limited to, E. coli, Salmonella spp., and Klebsiella spp.; bacteria from the phylum Bacteroidetes, including, but not limited to, Bacteroides spp. e.g., B. thetaiotaomicron, B. ovatus, B. fragilis, B. dorsei, B. diastonis, and B. vulgatus; Firmicutes bacteria including, but not limited to, Lactococcus and Lactobacillus spp., e.g., L. lactis, L. reuteri, L. casei, L. plantarum, and L. crispatus; Faecalibacterium spp.; Helicobacter spp.; Bacillus spp.; Streptococcus spp.; Staphylococcus spp.; Enterococcus spp.; Streptomyces spp.; Cyanobacter spp.; Campylobacter spp.; Clostridium spp.; Neisseria spp.; Moraxella spp.

In certain embodiments, the programmable endonuclease, e.g., Cas9, is used to select against non-engineered cells when the target host cell genome is actively replicating. By adding an inducer (e.g., aTc) to the cell culture, the programmable endonuclease will be expressed, bind the guide polynucleotide, e.g., sgRNA, and will only be able to cleave genomic DNA at the target site if recombination has not occurred. If cleavage occurs at the target site, the bacteria will die. Thus, bacteria that survive include the desired mutation and are easily harvested.

In embodiments where nickases are used, such as Cas9 nickases, that bind the genome but make only a single-strand break, the cell should not die when targeted by the guide polynucleotide/nickase complex, such as a sgRNA/nCas9 complex. Rather, one of several DNA repair pathways will be activated that result in opening the genome at the site of the ssDNA break, thereby enhancing genome editing efficiency. Expressing the nickase and guide polynucleotide will result in a ssDNA break to the bacterial genome. Subsequently expressed sgRNA/Cas9 complexes will continue making ssDNA breaks to the non-engineered genome but will have no effect on the engineered genome. Engineered cells will not be selected because a ssDNA break does not cause cell death. However, the efficiency of genomic engineering is significantly improved such that engineered bacteria can be screened via PCR rather than relying on selection via insertion of an antibiotic resistance gene.

Engineering efficiency can be measured as described in the Examples herein, by growing all cells on solid media after performing the gene editing protocols described herein. The number of cells that contain the engineered change divided by the number of total cells provides the percentage of correctly engineered cells. In normal recombineering conditions where no selection is occurring, efficiencies as high as 1-3% and as low as 0.001% (or 0% when no editing occurs) are typically achieved. Successful gene editing can be measured by performing diagnostic PCR that indicates whether or not a given colony contains the correct genome sequence. By “increasing the efficiency of genomic engineering or genome editing” as used herein is meant that recombineering frequencies of at least 5% are achieved, such as at least 10%, 15%, or more.

In certain embodiments, the nickase can be expressed with two guide polynucleotides, one that targets the programmable endonuclease to make a ssDNA break at the 5′ end of the target region, and one that targets the programmable endonuclease to make a ssDNA break at the 3′ end of the target region.

In additional embodiments, the programmable endonuclease has been mutated to lack endonuclease activity but is still able to tightly bind the target sequence when complexed with a guide polynucleotide (e.g., dCas9). When the dCas protein binds the target site, RNA polymerase is prevented from accessing the genome and mRNA transcription cannot occur. Hence, protein translation is prevented. Thus, the guide polynucleotide/dCas complex can be used to turn off specific genes in the bacterial genome.

The techniques described herein are broadly applicable and can provide for precise genome editing in diverse microorganisms. Using the single plasmid methods, any sequence in the host cell genome can be targeted. Thus, bacterial genomes can be manipulated to regulate gene expression, inactivate genes, repair genes, provide for efficient metabolic engineering, allow for bacterial strain typing, can be used to immunize cultures, can provide for autoimmunity or self-targeted cell killing, and for the engineering or control of metabolic pathways for improved biochemical synthesis.

While preferred embodiments of the present invention have been shown and described herein, it will be obvious to those skilled in the art that such embodiments are provided by way of example only. From the above description and the following Examples, one skilled in the art can ascertain essential characteristics of this invention, and without departing from the spirit and scope thereof, can make changes, substitutions, variations, and modifications of the invention to adapt it to various usages and conditions. Such changes, substitutions, variations, and modifications are also intended to fall within the scope of the present disclosure.

### EXPERIMENTAL

Aspects of the present invention are further illustrated in the following Examples. Efforts have been made to ensure accuracy with respect to numbers used (e.g., amounts, concentrations, percent changes, etc.) but some experimental errors and deviations should be accounted for. Unless indicated otherwise, temperature is in degrees Centigrade and pressure is at or near atmospheric. It should be understood that these Examples, while indicating some embodiments of the invention, are given by way of illustration only.

The following Examples are not intended to limit the scope of what the inventors regard as various aspects of the present invention.

### Example 1: Plasmid 1 Construction for In Vivo Genome Editing

A plasmid was constructed for use in in vivo genome editing as follows. The plasmid encoded Cas9 and a sgRNA complementary to a 20-bp target site in the E. coli tonA gene, operably linked to a PAM sequence. Paired together, the sgRNA and Cas9 protein were able to bind the target gene and induced a DSB at the target site. Because DSBs are toxic to most bacteria, and specifically E. coli, Cas9 activity causes cell death. Therefore, to prevent uncontrolled cell killing, Cas9 activity was controlled at the transcriptional, translational, and enzymatic levels by features of the single plasmid. In this Example, the enzymatic activity of Cas9 was reduced by the presence of AcrIIA4, an anti-CRISPR that binds to and inhibits Cas9 function. The ratio of the amount of AcrIIA4 to Cas9 protein determines the activity of Cas9. The amount of inhibition provided by AcrIIA4 was fine-tuned via modifications to the acrIIA4 promoter and ribosome binding site (RBS). The gene for AcrIIA4 was driven by a weak constitutive promoter, which resulted in constant, low-level expression of acrIIA4 Transcription of cas9 was under the control of an inducible promoter and translational activity was optimized by changes to the RBS. When the gene controlling Cas9 production was not induced, this allowed for enough AcrIIA4 production to inhibit Cas9 activity. However, when production of Cas9 was activated, more Cas9 was produced than could be bound and inhibited by AcrIIA4.

Plasmid 1 was constructed for performing specific genome editing with Cas9 in E. coli host cells and other organisms where delivery is achieved using electroporation or chemical transformation and where uninduced Cas9 activity may be high. The anti-CRISPR element was included to help keep uninduced Cas9 activity low. When Cas9 was used to perform genome editing, the enzyme generated a DSB, resulting in cell death. In this instance, the DSB caused by Cas9 must be repaired in order for cells to grow and divide. A repair template, composed of elements 3.1, 3.2, and 4, was provided in order to generate a specific genome edit.

Plasmid 1 was constructed using standard cloning methods. Sequence- and ligation-independent cloning (SLIC) was used to assemble parts from previously existing plasmids. Briefly, individual DNA sequence elements were cloned to produce a representative plasmid having Plasmid Element Organization A, depicted in FIG. 1. Exemplary plasmid component sequences are shown in Table 2. In FIG. 1, Element 1 is a transcriptional control unit (termed “Trxn Control” in FIG. 1) consisting of an inducible promoter. In this example, Element 1 included a repressor of the tet promoter, tetR, which also includes a promoter and RBS for generating TetR (SEQ ID NO:1), a tet promoter (SEQ ID NO:2), and a RBS (SEQ ID NO:3). Element 1 controls the transcription of Element 2, a cas9 gene (SEQ ID NO:4). Elements 3.1 and 3.2, upstream and downstream homology sequences, respectively, flanked Element 4. These sequences can vary in length between 10 to 2000 bps or more and are homologous to selected regions within a bacterial genome. The sequences were homologous to a region of the E. coli genome on either side of the gene tonA. The sequences for Element 3.1 and 3.2 used in this Example were SEQ ID NO:5 and SEQ ID NO:6, respectively. Element 4 was a representative donor polynucleotide (termed “Donor PN” in FIG. 1), which can vary between zero bases to thousands of bases in length, and can encode for a gene or genes to be inserted into a bacterial genome in order to provide a desired function or to produce a signal of a genomic change at the target locus. The presence of a donor PN is optional. If zero bases are present, the plasmid can be used to make deletions from the host genome. Examples of donor polynucleotides include, but are not limited to, genes that produce fluorescent proteins, antibiotics, entire metabolic pathways or portions thereof, entire enzymatic networks or portions thereof, to produce small molecules, and other heterologous gene products. Element 4 in this example included SEQ ID NO:7 and SEQ ID NO:8. Element 5 included a sgRNA and a promoter sequence, termed a “sgRNA unit.” The sgRNA element can contain one to multiple sgRNA units. Element 5 in this Example was one sgRNA unit that produced a sequence that targeted Cas9 to the tonA region of the E. coli genome, and is shown as SEQ ID NO:9 in Table 2. Element 6 was an origin of replication which enabled replication of the plasmid and segregation of replicated plasmids into daughter cells. The origin of replication was p15A (SEQ ID NO:10). Element 7 included a gene for an anti-CRISPR peptide, AcrIIA4 (SEQ ID NO:11), its associated promoter (SEQ ID NO; 13), and a RBS (SEQ ID NO:12). Element 8 was an antibiotic resistance gene (AbR), and, in this Example, was a chloramphenicol resistance gene (SEQ ID NO:14).

Because of the cloning technique chosen to construct Plasmid 1, the solution in which the plasmid was assembled contained salts that could reduce the efficiency of transforming the plasmid into cells by means of electroporation. Therefore, a transformation procedure was chosen that was more tolerant of the presence of salts. In this Example, plasmids were transformed via heat shock into 50 μl of chemically competent cells (Strain No. 1 of Table 3), were plated on selective antibiotic LB agar plates (Teknova Inc., Hollister, Calif.), and incubated overnight at 37° C. Resulting colonies were individually inoculated into selective antibiotic LB medium (Teknova Inc., Hollister, CA) and grown 12-16 hours with shaking at 37° C. Plasmids were purified from cultures of isolates using a QIAprep Miniprep Kit™ (Qiagen, Hilden, Germany), according to the manufacturer's instructions. Identity of the plasmid was confirmed by Sanger and next generation sequencing.

### Example 2: Plasmid 1 In Vivo Genome Editing

### Genome Editing

The purified plasmid from Example 1 was transformed into Strain No. 2 of Table 3 as follows. Between 50-100 ng of the plasmid were mixed with 50 μl of electrocompetent Strain No. 2 cells, electroporated, and recovered in 1 ml of SOC medium (super optimal broth) (Teknova Inc., Hollister, CA) with catabolite repression for 1 hour at 37° C. Recovered cells were plated on selective antibiotic LB agar plates and grown 12-16 hours at 37° C. Resulting colonies were referred to as the “single plasmid strain.” One colony of the single plasmid strain was selected to inoculate 5 ml of selective antibiotic LB medium and grown with shaking for 12-16 hours at 37° C. A volume of 100 μl of this culture was dispensed into well A1 of a 96-well plate, and 90 μl of LB medium was dispensed into wells B1-H1. The culture was serially diluted by mixing 10 μl from A1 into B1, 10 μl from B1 to C1, etc., until H1 had been mixed with 10 μl of G1. This resulted in a series of eight, 10-fold dilutions so that well H1 was diluted 10⁸ relative to well A1.

The cas9 gene, was under the control of a tetracycline (Tc) inducible promoter. As such, an analog of tetracycline, anhydrotetracycline (aTc, Clontech, Mountain View, Calif.) was used to induce cas9 expression. Using a multichannel pipette, 10 μl from A1-H1 was dispensed in a row near the top of an agar plate and allowed to drip down until it reached near the bottom of the plate. In this way, the single plasmid strain was drip-plated on both a LB-chloramphenical (LB-Cm) plate and a LB-Cm aTc plate (final concentration of aTc was 0.2 μg/ml). Plates were incubated for 12-16 hours at 37° C.

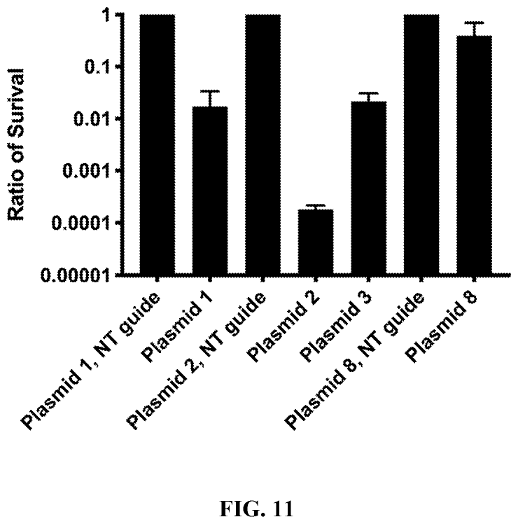

The number of colony forming units per milliliter (CFU/ml) plated was then calculated in the furthest dilution lane with growth exceeding 9 colonies on LB-Cm and LB-Cm aTc plates. The CFU/ml on the non-inducing plate was then divided by the CFU/ml on the inducing aTc plate to determine the ratio of bacteria killed as a result of cas9 induction versus uninduced expression of cas9. The ratios of cell survival results are summarized in FIG. 11. As shown in FIG. 11, plasmid 1 resulted in approximately 500× more killing when induced than the same plasmid with a sgRNA that did not target the E. coli genome (referred to herein as a “non-targeting guide” or “NT guide”).

### Phage Assay to Determine Bacterial Mutagenesis

Cells could have survived after induction of cas9 through targeted genome editing, or through non-specific mutations to cas9, the sgRNA, or the genome. To determine what proportion of cells that survived cas9 induction experienced targeted genome editing versus non-specific mutations, bacteria were assayed for disruption of the targeted gene. Expression of tonA enables T5 phage to infect and kill E. coli cells. If the targeted edit of the tonA gene occurred, the edited E. coli would survive incubation with T5 phage. To test this, 10 μl of 7×10¹⁰ plaque forming units per milliliter (PFU/ml) of T5 phage (ATCC, Old Town Manassas, Va.) were dripped vertically down an LB-kanamycin (LB-Kan) (LB: Teknova Inc., Hollister, CA; Kan: GoldBio, St. Louis, Mo.) agar plate and allowed to dry. Surviving colonies from the most dilute lanes of the killing assay were then struck perpendicularly across the T5 phage streak. If the streak of bacteria grew uninterrupted through the phage streak without thinning, the bacteria were determined to be resistant to T5 phage infection and likely experienced a targeted edit of the tonA gene as a result of the Cas9 enzyme and editing construct expressed from the plasmid.

### Colony PCR Assay

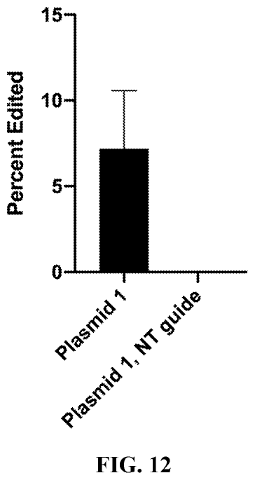

Patched colonies that grew uninterrupted through the phage streak were evaluated by colony PCR to determine if the provided donor DNA cassette was recombined into the target gene locus. All phage-resistant colonies were inoculated in 100 μl of LB medium and grown with shaking at 37° C. for 1 hour. The culture was then diluted 1:10 and boiled 5 minutes at 98° C. on a thermocycler. A volume of 1 μl of the boiled product was then used as template DNA for a PCR reaction. One forward primer that was complementary to a sequence upstream of where the donor DNA cassette was expected to insert (SEQ ID NO:15) was paired with a reverse primer complementary to one of the genes within the donor cassette (SEQ ID NO:16). Similarly, one reverse primer complementary to a sequence downstream of the desired insert location (SEQ ID NO:17) was paired with a forward primer complementary to another sequence within the donor DNA cassette (SEQ ID NO:18). Using these two pairs of primers in separate reactions with boiled genomic DNA, PCR was performed using Q5® High-Fidelity 2X Master Mix (New England Biolabs Inc., Ipswitch, Mass.), according to the manufacturer's instructions. The resulting products were evaluated by gel electrophoresis. If bands of the expected sizes from each primer pair were observed, this indicated successful homologous recombination of the donor DNA construct into the desired locus of the E. coli genome. Lack of a band from either or both PCR reactions indicated that the locus did not have the donor. Bands confirming donor DNA cassette recombination at the desired locus were tallied and compared to the original number of colonies assayed against T5 phage. Results are shown in FIG. 12 and show an average of approximately 7% editing.

### Example 3: Plasmid 2 Construction for In Vivo Genome Editing

A representative plasmid, having Plasmid Element Organization B (FIG. 2), was constructed essentially as described in Example 1, with the following modifications: site-directed mutagenesis was used to remove parts from a previously existing plasmid and individual DNA sequence elements were cloned to produce Plasmid 2. This plasmid contained all of the same elements and structure as shown in FIG. 1, Plasmid A, but lacked Elements 3.1, 3.2, and 4. The sequences of the remaining elements were the same as those indicated in Example 1. Plasmid 2 was constructed for performing non-specific genome deletions with Cas9 in E. coli and other organisms that can be transformed by electroporation or chemical transformation, and where uninduced Cas9 activity and recombination ability are high. The anti-CRISPR element was included to help keep uninduced Cas9 activity low. With Plasmid 2, Cas9 was still directed to a cut-specific sequence of the genome with an sgRNA, but the cell was not given a repair template (Elements 3.1, 3.2, and 4) with instructions on how to repair the DSB caused by Cas9. This resulted in either cell death from the DSB, genome rearrangement through recombination that left a large, variable, and non-specific deletion in the genome, removing the genomic sequence where Cas9 would bind. Organisms with high recombination ability can rearrange their genomes through this mechanism.

Plasmids were transformed, cultured and purified as described in Example 1. Editing efficiency is determined as described above.

### Example 4: Plasmid 2 In Vivo Genome Editing

### Genome Targeting

The in vivo gene editing plasmid in Example 3 was transformed into Strain No. 2 cells (Table 3) as outlined in Example 2. One colony of the single plasmid strain was inoculated and serially diluted as described in Example 2. Serial dilutions were drip-plated, CFU/ml were counted, and the amount of killing caused by Cas9 induction was calculated as described in Example 2. As shown in FIG. 11, Plasmid 2 results in approximately 5000× more killing than the same plasmid with a NT guide when induced.

### Example 5: Plasmid 3 Construction for In Vivo Genome Editing

Plasmid 3, another representative plasmid, having Plasmid Element Organization B (FIG. 2), was constructed essentially as described in Example 3, with the following modifications: SLIC was used to assemble parts from previously existing plasmids. The sequences were the same as those indicated in Example 3, with the exception of Element 2, which corresponded to SEQ ID NO:19. The plasmid component sequences are shown in Table 2. Plasmid 3 was constructed for performing specific genome editing in E. coli and other organisms with Cas9 that can be transformed using electroporation or chemical transformation, and where uninduced Cas9 activity may be high. The anti-CRISPR element was included to help keep uninduced Cas9 activity low. Compared to Plasmids 1 and 2, Plasmid 3 differed in the codon sequence of cas9. Plasmid 3 was constructed to compare whether codon sequence would significantly alter Cas9 activity and thus genome editing efficiency.

Plasmids were transformed via heat shock into 50 μl of chemically competent Strain No. 3 cells (Table 3) and were plated on selective antibiotic LB agar plates and incubated overnight at 37° C. Resulting colonies were individually inoculated into selective antibiotic LB medium and grown 12-16 hours with shaking at 37° C. Plasmids were purified from cultures of isolates using a Machery Nagel NucleoSpin™ Plasmid Kit (Machery-Nagel Inc., Bethlehem, Pa.), according to the manufacturer's instructions. Identity of the plasmid was confirmed by Sanger sequencing and next generation sequencing.

### Example 6: Plasmid 3 In Vivo Genome Editing

### Genome Editing

The in vivo gene editing plasmid in Example 5 was transformed into Strain No. 2 (Table 3) as outlined in Example 2. One colony of the single plasmid strain was inoculated and serially diluted as described in Example 2. Serial dilutions were drip-plated, CFU/ml counted, and the amount of killing caused by Cas9 induction was calculated as described in Example 2. As shown in FIG. 11, Plasmid 3 resulted in approximately 50× more killing than NT guide versions of similar plasmids. Although a NT guide version of Plasmid 3 was not constructed, other described versions of the single plasmid with NT guides did not exhibit killing. Accordingly, this likely holds true for Plasmid 3 as well.

### Example 7: Plasmid 4 Construction for In Vivo Genome Editing

Plasmid 4, another representative plasmid having Plasmid Element Organization A (FIG. 1), was constructed as follows. The plasmid encoded a Cas9 nickase (nCas9) and two sgRNAs complementary to two different 20-bp target sites in the E. coli tonA gene, each operably linked to a PAM sequence. Paired together, the sgRNAs and nCas9 were able to bind the bacterial genome in two places and induced two single-strand DNA breaks in the genome. In this Example, nCas9 production was controlled at the transcriptional, translational, and enzymatic levels as Cas9 was in Example 1. Plasmid 4 was constructed for performing specific genome editing in E. coli and other organisms with nCas9 that can be transformed using electroporation or chemical transformation, and where uninduced nCas9 activity may be high. The anti-CRISPR element was included to help keep uninduced nCas9 activity low. When nCas9 was used to perform genome editing, the enzyme generated a single-strand DNA break or “nick,” which did not result in cell death. The nick caused by nCas9 must be repaired in order for cells to grow and divide. A repair template composed of elements 3.1, 3.2, and 4 was provided to repair the host cell genome in order to generate a specific genome edit.

The in vivo gene targeting plasmid was constructed using standard cloning methods. SLIC was used to assemble parts from previously existing plasmids. Briefly, individual DNA sequence elements were cloned to produce a plasmid as depicted in FIG. 1. The plasmid component sequences are shown in Table 2. The order and sequence of the DNA elements was the same as in Plasmid 1 (Example 1) with the exception of Element 2 (the gene encoding the CRISPR enzyme) which was nCas9 (SEQ ID NO:20), and Element 5 (sgRNA), which was two tandem sgRNA units. Several different sgRNA units were tested and correspond to SEQ ID NO:21 (sgRNA pair unit #1); SEQ ID NO:22 (sgRNA pair Unit #2); and SEQ ID NO:23 (sgRNA pair unit #3).

Plasmids were transformed, purified, and verified as described in Example 5.

### Example 8: Plasmid 4 In Vivo Genome Editing

### Genome Editing

The sequence of the in vivo gene editing plasmid from Example 7 was transformed into Strain No. 2 (Table 3) as described in Example 2. One colony of the single plasmid strain was selected to inoculate 5 ml of LB-Cm medium and grown with shaking for 12-16 hours at 37° C. This culture was referred to as the “overnight culture.” The gene encoding nCas9 was under the control of a tetracycline (Tc)-inducible promoter. As such, aTc was used to induce Cas9 expression. A volume of 6 μl of the overnight culture was back diluted (1:500) into 3 ml of LB-Kan medium and into 3 ml of LB-Kan aTc medium (final concentration of aTc was 0.2 μg/ml) and grown with shaking for 7 hours at 37° C. These cultures were referred to as the “first back-dilution cultures.” A volume of 6 μl of each first back-dilution culture was back-diluted again (1:500) into 3 ml of the same media types, LB-Kan or LB-Kan aTc. These were then grown with shaking for 12-16 hours at 37° C. and were referred to as the “second back-dilution cultures.”

A volume of 100 μl of each second back-dilution culture was dispensed into separate wells in row A of a 96-well plate (A1 and A2), and 90 μl of LB medium was dispensed into all 7 remaining column wells below (B1-H1 and B2-H2). The culture was serially diluted by mixing 10 μl from row A into 90 μl of LB in row B (A1 into B1 and A2 into B2), 10 μl from row B into 90 μl of LB in row C (B1 into C1, B2 to C2) etc., until H1 had been mixed with 10 μl of G1 and H2 had been mixed with 10 μl of G2. This resulted in a series of eight 10-fold dilutions so that well H1 was diluted 10⁸ relative to well A1.

Using a multichannel pipette, 10 μl from each column of wells (A1-H1 and A2-H2) was dispensed in a row near the top of individual agar plates and allowed to drip down until it reached near the bottom of the plate. In this way, the second back-dilution cultures (induced and non-induced) of the nCas9 single plasmid strain were each drip-plated on both a LB-Kan plate and a LB-Kan aTc plate. Plates were incubated for 12-16 hours at 37° C.

The number of colony forming units per milliliter (CFU/ml) plated was then calculated in the furthest dilution lane with growth exceeding 9 colonies on LB-Kan and LB-Kan aTc plates. The CFU/ml on the non-inducing plate was then divided by the CFU/ml on the inducing aTc plate to determine the ratio of surviving bacteria after nCas9 induction.

### Phage Assay to Determine Bacterial Mutagenesis

Cells could have survived after induction of nCas9 through targeted genome editing or through non-specific mutations to nCas9, the sgRNA, or the genome. Single-strand DNA breaks could have been repaired by the cell, but nCas9 would have continued to induce single-strand DNA breaks at that same site. To determine what proportion of cells that survived nCas9 induction experienced targeted genome editing vs. non-specific mutations, bacteria were assayed for disruption of the targeted gene as described in Example 2.

### Colony PCR Assay

Patched colonies that grew uninterrupted through the phage streak were evaluated by colony PCR to determine if the provided donor DNA cassette was recombined into the target gene locus. All phage-resistant colonies were inoculated in 100 μl of LB medium. The culture was then boiled 5 minutes at 98° C. on a thermocycler. A volume of 2 μl of the boiled product was then used as template DNA for a PCR reaction. One forward primer that was complementary to a sequence upstream of where the donor DNA cassette was expected to insert (SEQ ID NO:15) was paired with a reverse primer complementary to a sequence downstream of the desired insert location (SEQ ID NO:17). Using these primers and boiled genomic DNA template, PCR was performed using Q5® High-Fidelity 2× Master Mix (New England Biolabs Inc., Ipswitch, Mass.) according to the manufacturer's instructions. The resulting products were evaluated by gel electrophoresis. Lack of a band indicated that the locus did not have the insert. Presence of a band did not differentiate presence of the insert from the native genome sequence as both products were of sizes indistinguishable by agarose gel electrophoresis. As such, PCR products were further evaluated by Sanger sequencing using SEQ ID NO:15 and SEQ ID NO:17 as individual sequencing primers.

qPCR Assay

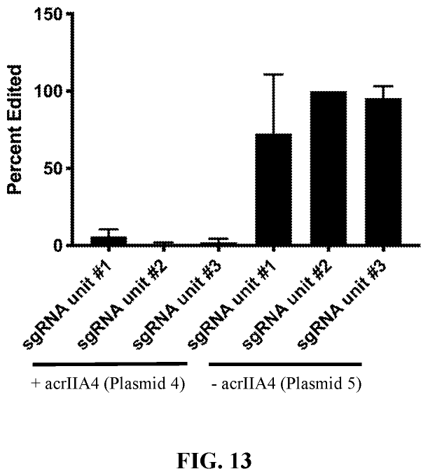

Patched colonies that grew uninterrupted through the phage streak were evaluated by qPCR to determine if the provided donor DNA cassette was recombined into the target gene locus. Phage-resistant colonies were inoculated in 100 μl of LB medium. The resulting culture was boiled 5 minutes at 98° C. on a thermocycler. A volume of 2 μl of the boiled sample was used as template DNA for a qPCR reaction. The same forward primer (SEQ ID NO:15) and reverse primer (SEQ ID NO:17) used in colony PCR were paired for qPCR. Both primers were mixed in a 1:1:1 ratio with one of two FAM TaqMan™ (Thermo Fisher Scientific, Waltham, Mass.) probes: one was complementary to the donor DNA (SEQ ID NO:24) and the other was complementary to the target gene (SEQ ID NO:25). Primer, probes, and template were mixed with 2× TaqMan™ Fast Advanced Master Mix (Thermo Fisher Scientific, Waltham, Mass.) to a final volume of 20 μl. Reactions were set up in a 96-well plate and evaluated on a StepOnePlus™ Real-Time PCR System (Applied Biosystems, Foster City, Calif.). Presence of signal from the donor DNA probe indicated recombination had occurred, while presence of signal from the target DNA probe indicated recombination had not occurred. Positive signal was defined as signal with a mean C_{T} of less than 35 cycles, where C_{T} was the cycle number at which 50% of the fluorescence intensity maximum was reached. Results for the percent of qPCR-confirmed edited cells are shown in FIG. 13. All three versions of Plasmid 4 with the varying sgRNA units are represented in FIG. 13 as “+acrIIA4.” As shown in FIG. 13, average percent editing for each of the sgRNA units in Plasmid 4 was between 0.7% and 5.9%.

### Example 9: Plasmid 5 Construction for In Vivo Genome Editing

A representative plasmid, Plasmid 5, having Plasmid Element Organization C (FIG. 3), was constructed as follows. Plasmid 5 was constructed as described in Example 7 with the exception that enzymatic control through anti-CRISPR activity was excluded. Plasmid 5 was constructed for performing specific genome editing in E. coli and other organisms with nCas9 that can be transformed using electroporation or chemical transformation, and where induced nCas9 activity may be low. The anti-CRISPR element was excluded so that induced nCas9 activity would be high.

The in vivo gene editing plasmid was constructed using site-directed mutagenesis to remove a sequence from previously existing plasmids. Briefly, individual DNA sequence elements were cloned to produce a plasmid as depicted in FIG. 3. The plasmid component sequences are shown in Table 2. The order and sequence of the DNA elements were the same as Plasmid 4 with the exception that Element 7 (anti-CRISPR) was removed.

Plasmids were transformed, purified, and verified as described in Example 5.

### Example 10: Plasmid 5 In Vivo Genome Editing

### Genome Editing

The sequence of the in vivo gene editing plasmid from Example 9 was transformed into Strain No. 2 (Table 3) as described in Example 2. One colony of the nCas9 single plasmid strain from Example 9 was selected to inoculate 5 ml of LB-Kan medium, grown and back diluted as described in Example 8. Each back-diluted sample was serially diluted as described in Example 8 and each serially diluted sample was plated as described in Example 8. Colonies were counted and calculated as described in Example 8.

### Phage Assay to Determine Bacterial Mutagenesis

Bacteria were assayed for disruption of the targeted gene as described in Example 2.

qPCR Assay

Patched colonies that grew uninterrupted through the phage streak were evaluated by qPCR as described in Example 8. Results for the percent of qPCR-confirmed edited cells are shown in FIG. 13. All three versions of Plasmid 5 with the varying sgRNA units (sgRNA unit pair #1, sgRNA unit pair #2, and sgRNA unit pair #3) are represented in FIG. 13 as “−acrIIA4.” Average percent editing for each of the sgRNA units in Plasmid 5 was between 73% and 100%.

### Example 11: Plasmid 6 Construction for In Vivo Genome Binding

Plasmid 6, another representative plasmid having Plasmid Element Organization B (FIG. 2), was constructed for use in in vivo genome binding as follows. The plasmid encoded a catalytically inactive Cas9 (dCas9) and four sgRNAs complementary to four different 20-bp target sites in the E. coli genome, each operably linked to a PAM sequence. The four sgRNAs were complementary to regions in the following genes: flhC, gfp, lacZ, and gusA. Paired together, the sgRNAs and dCas9 protein were able to bind the target genes and repress transcription at the target sites. dCas9 activity was controlled at the transcriptional, translational, and enzymatic levels by features of the single plasmid in order to limit unintended activity. In this Example, the DNA binding activity of dCas9 was reduced by the presence of anti-CRISPR AcrIIA4, an anti-CRISPR that binds to and inhibits dCas9 function. The ratio of the amount of AcrIIA4 to dCas9 protein determined the activity of dCas9. Therefore, the amount of inhibition provided by AcrIIA4 was fine-tuned via modifications to the acrIIA4 promoter and ribosome binding site (RBS). The gene for AcrIIA4 was driven by a weak constitutive promoter, which resulted in constant, low-level expression of acrIIA4. Transcription of dCas9 was under the control of an inducible promoter and translational activity was optimized by changes to the RBS. When the gene controlling dCas9 production was not induced, this allowed for enough AcrIIA4 production to inhibit dCas9 activity. However, when production of dCas9 was activated, more dCas9 was produced than could be bound and inhibited by AcrIIA4.

The in vivo gene binding plasmid was constructed using standard cloning methods. SLIC was used to assemble parts from previously existing plasmids. Briefly, individual DNA sequence elements were cloned to produce a plasmid as depicted in FIG. 2. The plasmid component sequences are shown in Table 2. The sequences of the elements of Plasmid 6 were the same as those indicated in Example 5, with the following exceptions: Element 2 was dCas9 (SEQ ID NO:26); and Element 5 sgRNA encoded four sgRNA units (SEQ ID NO:27). Plasmid 6 was constructed for performing specific genome binding in E. coli and other organisms that can be transformed using electroporation or chemical transformation, and where uninduced dCas9 activity may be high. The anti-CRISPR element was included to help keep uninduced dCas9 activity low. When dCas9 was used to perform genome binding, the enzyme did not generate any DNA breaks, but bound tightly to the genome at a site prescribed by the sgRNA. This did not result in cell death. Binding to the genome at specific sites enabled blocking or activating transcription of a specific gene or group of genes without permanently altering the genome. Multiple sgRNA sequences were included to demonstrate that multiple genes were able to be controlled simultaneously.

Plasmids were transformed, purified, and verified as described in Example 5, with the exception that plasmids were transformed into Strain No. 4 (Table 3).

### Example 12: Plasmid 6 In Vivo Genome Binding

### Genome Repression

The in vivo gene binding plasmid described in Example 11 was transformed into Strain No. 2 and Strain No. 5 (Table 3) as follows. Between 10-100 ng of the single plasmid were mixed with 50 μl of electrocompetent cells, electroporated, and recovered in 1 ml of SOC medium for 1 hour at 37° C. Recovered cells were plated on LB-Cm agar plates and grown 12-16 hours at 37° C. Resulting colonies in Strain No. 2 were tested for flhC activity and were referred to as the “dCas9 single plasmid tonA+ strain.” Resulting colonies in Strain No. 5 were tested for lacZ, gusA, and gfp activity and referred to as the “dCas9 single plasmid gfp+ strain.”

One colony of the dCas9 single plasmid tonA+ strain or gfp+ strain (depending on the intended assay) was selected to inoculate 3 ml of selective antibiotic medium and was grown with shaking for 12-16 hours at 37° C. The gene encoding dCas9 was under the control of a Tc-inducible promoter. As such, aTc was used to induce dCas9 expression. After 12-16 hours of growth, the culture was back diluted 1:100 in 3 ml of selective antibiotic medium and 3 ml of selective antibiotic medium with aTc and grown with shaking at 37° C. for 5 to 6 hours. The optical density of each culture after 5 to 6 hours of induction was measured at a wavelength of 600 nm. Cultures were accordingly diluted to an optical density that would result in about 100 CFU/ml, and 100 μl of this was plated on assay-appropriate plates and grown 12-16 hours at 37° C.

The plates and media used were appropriate for the phenotypic assays being performed to detect gene expression of lacZ, gusA, flhC, and gfp. The LacZ blue white screening assay was performed as previously described (see Vieira, et al., Gene (1982) 19:259-268). The GusA assay followed the same concept as the LacZ blue white screening assay and utilized X-Gluc (5-bromo-4-chloro-3-indolyl-β-D-glucuronide) for detection of enzyme activity (Frampton, et al., J. Food Protect. (1988) 51:402-404). A standard swimming motility assay was utilized to detect repression of FlhC (see Gomez-Gomez, et al., BMC Biology (2007) 5:14). Plates were imaged on an Azure Biosystems™ c600 (Dublin, Calif.) to allow detection and to count GFP fluorescent colonies.

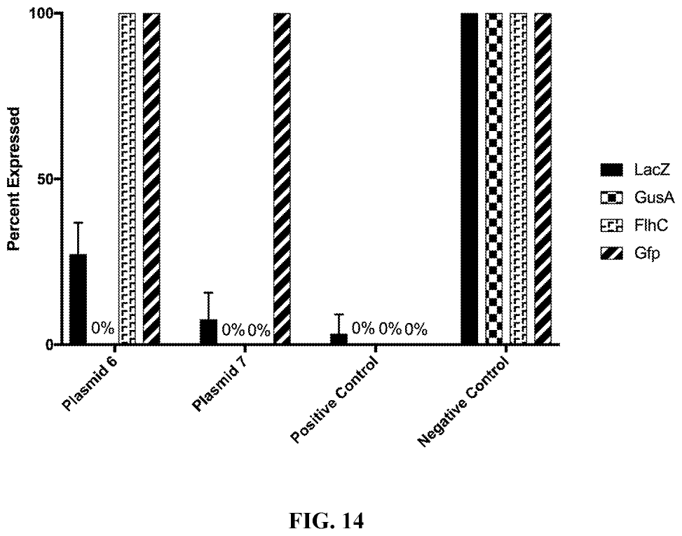

Results are shown in FIG. 14. As seen in FIG. 14, expression of dCas9 from Plasmid 6 resulted in partial repression of the lacZ (27.3% average expression), complete repression of the gusA (0% average expression), no repression of flhC (100% average expression), and no repression of gfp, (100% average expression). Expression of dCas9 from the positive control strain resulted in nearly complete repression of each target gene. The low level of LacZ expression from one test of the positive control strain was considered part of the noise of the assay, as all colonies will turn blue over time, so less than optimal timing of imaging can allow for spurious signal to arise. Expression of dCas9 from the negative control strain did not repress expression of any of the target genes.

### Example 13: Plasmid 7 Construction for In Vivo Genome Binding

A representative plasmid, Plasmid 7, having Plasmid Element Organization D (FIG. 4), was constructed as follows. This plasmid was constructed for use in in vivo genome binding as described in Example 11, with the exception that enzymatic control through anti-CRISPR activity was excluded. Plasmid 7 was constructed for performing specific genome binding in E. coli and other organisms with dCas9 that can be transformed using electroporation or chemical transformation, and where induced dCas9 activity may be low. The anti-CRISPR element was excluded so that induced dCas9 activity would be high. Multiple sgRNA sequences were included to demonstrate that multiple genes were able to be controlled simultaneously.

The in vivo gene binding plasmid was constructed using standard cloning methods. Site-directed mutagenesis was used to remove an element from a previously existing plasmid resulting in a plasmid with the structure depicted in FIG. 4. This plasmid contained all of the same elements and structure as Plasmid 6, but lacked the anti-CRISPR Element 7. The plasmid component sequences are shown in Table 2. The sequences of the present elements were the same as those indicated in Example 11.

Plasmids were transformed, purified, and verified as described in Example 1.

### Example 14: Plasmid 7 In Vivo Genome Binding

### Genome Repression

The in vivo gene binding plasmid described in Example 13 was transformed into Strain No. 5 (Table 3) as described in Example 12. One colony of the dCas9 single plasmid gfp+ strain was cultured, induced, diluted, and plated as described in Example 12. Phenotypic assays were performed, and the associated plates and media were used as described in Example 12. Results are shown in FIG. 14. As can be seen, removal of anti-CRISPR from Plasmid 6 to make Plasmid 7 allowed for greater dCas9 activity, and accordingly, stronger repression was observed at the lacZ and flhC loci. In summary, expression of dCas9 from Plasmid 7 resulted in nearly complete repression of the lacZ (11.5% average expression), complete repression of the gusA (0% average expression), complete repression of flhC (0% average expression), and no repression of gfp (100% average expression). Continued expression of Gfp was attributed to gfp being an exogenous gene under the control of a strong exogenous promoter, thereby making it more difficult to silence. As described in Example 12, positive and negative controls performed as expected.

### Example 15: Plasmid 8 Construction for In Vivo Genome Editing

Plasmid 8, another representative plasmid having Plasmid Element Organization B (FIG. 2), was constructed for use in in vivo genome editing as described in Example 1. The plasmid component sequences are shown in Table 2. The sequences were the same as those indicated in Example 5 (Plasmid 3), with the exception that the anti-CRISPR gene present, Element 7, was acrIIA2 (SEQ ID NO:41). Plasmid 8 was constructed for performing non-specific genome deletions in E. coli and other organisms with Cas9 that can be transformed using electroporation or chemical transformation, where uninduced Cas9 activity and recombination ability may be high. The anti-CRISPR element was included to help keep uninduced Cas9 activity low. The anti-CRISPR gene element present was acrIIA2 in order to demonstrate that diverse anti-CRISPR peptides could be used to optimize CRISPR-nuclease activity. With Plasmid 8, Cas9 was still directed to a cleave a specific sequence of the genome with a sgRNA, but the cell was not given a repair template (Elements 3.1, 3.2, and 4) so repair of the DSB caused by Cas9 did not occur. This resulted in either cell death from the DSB, or the rearrangement of the host cell genome through recombination leaving a large, variable, and non-specific deletion in the genome to cause removal of the genomic sequence where Cas9 would bind. Organisms with high recombination ability can rearrange their genomes through this mechanism.

Plasmids were transformed, purified, and verified as described in Example 5.

### Example 16: Plasmid 8 In Vivo Genome Editing

### Genome Editing

The in vivo gene editing plasmid in Example 15 was transformed as outlined in Example 2 into Strain No. 2 (Table 3). One colony of the single plasmid strain was inoculated and serially diluted as described in Example 2. Serial dilutions were drip-plated, CFU/ml were counted, and the amount of killing caused by Cas9 induction was calculated as described in Example 2. As seen in FIG. 11, Plasmid 8 resulted in an average of 7× more killing than the same plasmid with a NT guide when induced.

### Example 17: Plasmid 9 Construction for In Vivo Genome Editing

A representative plasmid, Plasmid 9, having Plasmid Element Organization E (FIG. 5), was constructed as follows. This in vivo genome editing plasmid can be delivered by conjugation to gut bacteria. The plasmid encoded Cas9 and a sgRNA complementary to a 20-bp target site in the Bacteroides tdk gene, operably linked to a PAM sequence. Paired together, the sgRNA and Cas9 protein were able to bind the target gene and induce a DSB at the target site. Because DSBs are toxic to most bacteria, and specifically Bacteroides, Cas9 activity was controlled at the transcriptional and translational levels by features of the single plasmid in order to limit unintended activity. Transcription of cas9 was under the control of an inducible promoter and translational activity was optimized by changes to the RBS. Plasmid 9 was constructed for performing non-specific genome deletions with Cas9 in Bacteroides thetaiotaomicron and other organisms that can be transformed using conjugation, and where uninduced Cas9 activity, and recombination ability may be high. The anti-CRISPR element was included to help keep uninduced Cas9 activity low. With Plasmid 9, Cas9 was still directed to cleave a specific sequence of the genome with a sgRNA, but the cell was not given a repair template (Elements 3.1, 3.2, and 4) that would have provided instructions on how to repair the DSB caused by Cas9. This resulted in either cell death from the DSB, or the rearrangement of the host cell genome through recombination. This left a large, variable, and non-specific deletion in the genome, which removed the genomic sequence where Cas9 would bind. Organisms with high recombination ability can rearrange their genomes through this mechanism.

The in vivo gene editing plasmid was constructed using standard cloning methods. SLIC was used to assemble parts from previously existing plasmids. Briefly, individual DNA sequence elements were cloned to produce a representative plasmid having the plasmid element organization as shown in FIG. 5. The plasmid component sequences are shown in Table 2. Referring to FIG. 5, Element 1 was a transcriptional control unit consisting of an inducible promoter. A repressor of the tet promoter, tetR, which also includes a promoter and ribosome binding site (RBS) for generating TetR, a tet promoter, a RBS following the tet promoter, and a cas9 promoter were used (SEQ ID NOS:28-31, respectively). Element 1 controlled the transcription and translation of Element 2, a gene encoding a Cas9 (SEQ ID NO:32). Elements 3.1 and 3.2 were upstream and downstream homology sequences, respectively, and flanked Element 4. These sequences can vary in length between 10-2000 or more bps and are homologous to regions within a bacterial genome. The sequences were homologous to a region of the B. thetaiotaomacron genome on either side of the gene tdk. The sequences for Element 3.1 and 3.2 used in this Example were (SEQ ID NO:35 and SEQ ID NO:36, respectively). Element 4 is optional, and is a donor PN element. If present, the donor PN can be one to thousands of bases in length and can encode for a gene or genes to be inserted into a bacterial genome, such as to provide a desired function, or to produce a signal of a genomic change at the target locus. If absent, the plasmid can be used to make targeted deletions from the host genome. Examples of donor genes include, but are not limited to, genes that produce fluorescent proteins, antibiotics, parts of or entire metabolic pathways, parts of or entire enzymatic networks to produce small molecules, and other heterologous gene products. In this case, Element 4 was absent. Element 5 was a sgRNA element, which included a promoter and sgRNA sequence, termed a “sgRNA unit.” The sgRNA element can contain one to multiple sgRNA units. Element 5 in this example included one sgRNA unit (SEQ ID NO:38) that produced a sequence targeting Cas9 to the tdk region of the B. thetaiotaomacron genome. Element 6 was an origin of replication and in this example was found in two places in the plasmid. Origin #1 (SEQ ID NO:37) allowed for replication in Bacteroides, and Origin #2 (SEQ ID NO:34) allowed for replication in E. coli. Element 8 was a AbR cassette. Two AbR cassettes were present in this Example. AbR #1 (SEQ ID NO:39) allowed for selection in Bacteroides and the AbR #2 (SEQ ID NO:40) allowed for selection in E. coli. Element 9 was the origin of transfer (SEQ ID NO:33) and allowed for conjugation of the plasmid to occur in Bacteroides.

Plasmids were transformed, purified, and verified as described in Example 5 with the exception that plasmids were transformed into Strain No. 6 (Table 3).

### Example 18: Plasmid 9 In Vivo Genome Editing

### Genome Editing

After purification of Plasmid 9 described in Example 17, the plasmid was transformed into Strain No. 7 (Table 3) as follows. Between 20-50 ng of the single plasmid were mixed with 50 μl of electrocompetent Strain No. 8 cells (Table 3), electroporated, and recovered in 1 ml of super optimal broth with SOC medium containing 0.3 mM 2,6-Diaminoheptanedioic acid (DAP, Sigma-Aldrich Corp., St Louis, Mo.) for 1 hour at 37° C. Recovered cells were plated on selective antibiotic LB agar plates supplemented with 0.3 mM DAP (2,6-Diaminoheptanedioic acid, Sigma-Aldrich Corp., St Louis, Mo.) and grown 16-20 hours at 37° C. Resulting colonies were referred to as the “single plasmid conjugation strain.”

Plasmid 9 from the single plasmid conjugation strain was conjugated into Bacteroides thetaiotaomicron Strain No. 8 (Table 3) as follows. Overnight cultures of B. thetaiotaomicron and the single plasmid conjugation strain were diluted back and grown to an OD₆₀₀ of 0.2-0.3 and 0.5-0.7, respectively. B. thetaiotaomicron was added to the single plasmid conjugation strain at a ratio of 5:1 (v/v). The mating mixture was pelleted, resuspended in 20 μl of BHI (Brain Heart Infusion, VWR International, Pittsburgh, Pa.) media supplemented with 5 mg/l hemin (Sigma-Aldrich Corp., St Louis, Mo.) and 1 g/l L-cysteine (Sigma-Aldrich Corp., St Louis, Mo.) (BHIS media), spotted onto a BHI agar plate and incubated aerobically at 37° C. for 16-20 hours. Cells were then collected by scraping, resuspended in 1 ml BHIS, and drip-plated as 1:10 serial dilutions as above with the following differences: on BHI agar plates containing 200 μg/ml gentamicin (Gm), 200 μg/ml Gm and 25 μg/ml erythromycin (Erm), and 200 μg/ml Gm, 25 μg/ml Erm and 100 ng/ml of aTc. Plates with aTc were included in order to induce expression of cas9. Plates were incubated anaerobically for 2 days at 37° C.

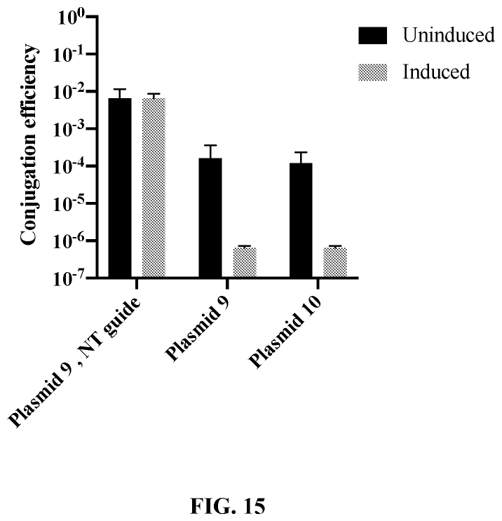

The CFU/ml plated were calculated in the furthest dilution lane with growth exceeding 9 colonies on BHI Gm, BHI Gm Erm and BHI Gm Erm aTc plates. The CFU/ml on the BHI Gm Erm and BHI Gm Erm aTc plates were divided by the CFU/ml on the BHI Gm plate to determine the conjugation efficiency with and without cas9 induction. The difference in conjugation efficiency upon induction conditions between Plasmid 9 and the same plasmid with a sgRNA that does not target the B. thetaiotaomicron genome corresponds to the Cas9-induced cell killing. The conjugation efficiency results are summarized in FIG. 15. Plasmid 9 results in ˜250× more killing when induced than the same plasmid with a sgRNA that does not target the B. thetaiotaomicron genome (referred to herein as a “non-targeting guide” or “NT guide”).

### Colony PCR Assay

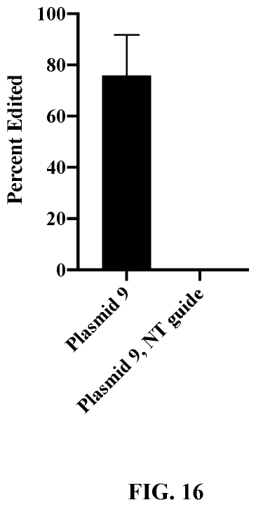

Resulting single colonies that grew in BHI Gm Erm aTc plates were evaluated by colony PCR to determine if the provided donor polynucleotide cassette was recombined into the target gene locus. Colonies were re-patched in BHI Gm Erm plates and inoculated in 50 μl of Alkaline lysis buffer (25 mM NaOH and 0.2 mM EDTA in dH₂O) in PCR tubes. These were incubated in a thermocycler at 95° C. for 30 min. A volume of 16 μl was transferred to a clean PCR tube containing 144 μl of dH₂O and 16 μl of neutralization buffer (40 mM Tris-HCl pH 7 in dH₂O). From each cell lysate, 5 μl were added to a PCR reaction consisting of 1× Q5® High-Fidelity 2× Master Mix (New England Biolabs Inc., Ipswitch, Mass.), 0.5 μM of forward primer complementary to a sequence upstream of the target gene locus (SEQ ID NO:42), 0.5 μM of reverse primer complementary to a sequence downstream of the desired target location (SEQ ID NO:43), 5 μl of cell lysate and nuclease-free water up to 25 μl. PCR tubes were transferred to a PCR machine for routine PCR according to the manufacturer's instructions. The resulting products were evaluated by gel electrophoresis. If the PCR reaction was successful, the primer pair generated either the band size corresponding to the successful homologous recombination of the insert DNA construct into the B. thetaiotaomicron genome, or the band size corresponding to the non-edited locus. Results are shown in FIG. 16. As can be seen, this resulted in an editing efficiency of 60.0% to 91.7%.

### Example 19: Plasmid 10 Construction for In Vivo Genome Editing

A representative plasmid, Plasmid 10, having Plasmid Element Organization F (FIG. 6,) was constructed as described in Example 17.

The in vivo gene editing plasmid was constructed using standard cloning methods. SLIC was used to assemble parts from previously existing plasmids. Briefly, individual DNA sequence elements were cloned in to produce a plasmid as depicted in FIG. 6. This plasmid contained all of the same element types and structure as FIG. 5 except the plasmid lacked Elements 3.1, 3.2, and 4. The plasmid component sequences are shown in Table 2. The sequences of the elements present in Plasmid 10 were the same as those indicated in Example 17. Plasmid 10 was constructed for performing non-specific genome deletions with Cas9 in Bacteroides thetaiotaomicron and other organisms that can be transformed using conjugation, and where induced Cas9 activity may be low, and recombination ability may be high. The anti-CRISPR element was excluded so that induced Cas9 activity would be high. With Plasmid 10, Cas9 was still directed to a cut-specific sequence of the genome with a sgRNA, but the cell was not given a repair template (Elements 3.1, 3.2, and 4) with instructions on how to repair the DSB caused by Cas9. This resulted in either cell death from the DSB, or the rearrangement of the host cell genome through recombination leaving a large, variable, and non-specific deletion in the genome, which removed the genomic sequence where Cas9 would bind. Organisms with high recombination ability can rearrange their genomes through this mechanism.

Plasmids were transformed, purified, and verified as described in Example 5 with the exception that plasmids were transformed into Strain No. 7 (Table 3).

### Example 20: Plasmid 10 In Vivo Genome Editing

### Genome Targeting

The in vivo gene editing plasmid in Example 19 was transformed into Strain No. 7 cells (Table 3) as outlined in Example 18. A colony of single plasmid Strain No. 7 was used to conjugate Plasmid 10 into Bacteroides thetaiotaomicron Strain No. 8 (Table 3) as outlined in Example 18. CFU/ml were counted, and the amount of killing caused by Cas9 induction was calculated as described in Example 18. As shown in FIG. 15, Plasmid 10 resulted in 185× more killing than the related Plasmid 9 with a NT guide when induced.

### Example 21 Plasmid 11 Construction for In Vivo Genome Editing

A representative plasmid, Plasmid 11, essentially having Plasmid Element Organization G (FIG. 7), is constructed in order to perform genome editing in Firmicutes microbes, such as Lactobacillus spp. and Lactococcus spp. This in vivo genome editing plasmid is suitable for delivery by electroporation. The plasmid encodes Cas9 and a sgRNA complementary to a 20-bp target site in a Lactobacillus gene, operably linked to a PAM sequence. Paired together, the sgRNA and Cas9 protein bind the target gene and induce a DSB at the target site. Because DSBs are toxic to most bacteria, and specifically Lactobacillus, Cas9 activity can be controlled at the transcriptional and translational levels by features of the single plasmid in order to limit unintended activity. Transcription of cas9 is driven by a constitutive promoter and translational activity can be optimized by changes to the RBS. In this Example, the enzymatic activity of Cas9 can be reduced by the presence of AcrIIA4, an anti-CRISPR that binds to and inhibits Cas9 function.

The in vivo gene editing plasmid is constructed using standard cloning methods. Briefly, individual DNA sequence elements are cloned to produce a representative plasmid having the plasmid element organization as shown in FIG. 7. The plasmid component sequences are shown in Table 1. Referring to FIG. 7, Element 2 is a gene encoding Cas9 (SEQ ID NO:44). Elements 3.1 and 3.2 are upstream and downstream homology sequences, respectively. These sequences can vary in length between 10 to thousands of bps and are homologous to regions within a bacterial genome. In this Example, the sequences are homologous to a region of the Lactobacillus paracasei genome on either side of the gene LSEI_2368 (SEQ ID NO:45), but can be homologous to any other desired target regions in the Lactobacillus paracasei genome. The sequences for Elements 3.1 and 3.2 used in this Example are (SEQ ID NO:46 and SEQ ID NO:47, respectively). Element 4 is a donor polynucleotide and can be one base to thousands of bases in length and can encode a gene or genes to be inserted into a bacterial genome such as to provide a function, or to produce a signal of a genomic change at the target locus. The presence of a donor PN is optional. If zero bases are present, the plasmid can be used to make targeted deletions from the host genome. Examples of donor genes include, but are not limited to, genes that produce fluorescent proteins, antibiotics, parts of or entire metabolic pathways, parts of or entire enzymatic networks to produce small molecules, and other heterologous gene products. Element 5 is a sgRNA element, which includes a promoter and sgRNA sequence, termed a “sgRNA unit.” The sgRNA element can contain one to multiple sgRNA units. Element 5 in this example includes one sgRNA unit (SEQ ID NO:48) that produces a sequence targeting Cas9 to the LSEI_2368 region of the Lactobacillus paracasei genome. Element 6 is an origin of replication and in this example is found in two places in the plasmid. Origin #1 (SEQ ID NO:49) allows for replication in Lactobacillus, and Origin #2 (SEQ ID NO:50) allows for replication in E. coli. The E. coli origin can be omitted when cloning is performed using a strain such as Lactococcus lactis. Element 7 includes a gene for an anti-CRISPR peptide, here acrIIA4 (SEQ ID NO:51). Element 8 is a AbR cassette. Two AbR cassettes are present in this Example. AbR #1 (SEQ ID NO:52) allows for selection in Lactobacillus and the AbR #2 (SEQ ID NO:53) allows for selection in E. coli. The E. coli AbR cassette can be omitted when cloning is performed using a strain such as Lactococcus lactis.

Plasmids can be transformed, purified, and verified as described in Example 5 with the exception that plasmids are transformed into Strain No. 9 (Table 2).

### Example 22: Plasmid 12 Construction for In Vivo Genome Editing

A representative plasmid, Plasmid 12, having Plasmid Element Organization H (FIG. 8), is constructed as described in Example 21. Paired together, the sgRNA and Cas9 protein are able to bind the target gene and induce a DSB at the target site. Because DSBs are toxic to most bacteria, and specifically Lactobacillus, Cas9 activity causes cell death. Therefore, to prevent uncontrolled cell killing, Cas9 activity is controlled at the transcriptional, translational, and enzymatic levels by features of the single plasmid. In this Example, the enzymatic activity of Cas9 is reduced by the presence of AcrIIA4, an anti-CRISPR that binds to and inhibits Cas9 function. If the gene controlling Cas9 production is not induced, this allows for enough AcrIIA4 production to inhibit Cas9 activity. However, if production of Cas9 is activated, more Cas9 is produced than can be bound and inhibited by AcrIIA4.

This plasmid contains all of the same element types and structure as FIG. 7 except it also contains Element 1, a transcriptional control unit (termed “Trxn Control” in FIG. 8) consisting of an inducible promoter. In this example, Element 1 can also include an inducible element that responds to carbohydrates, peptides, other metabolites or other environmental signals, such as the spp-derived two-component transcriptional activator (SEQ ID NO:54), to control the activity of a promoter such as the P_{sppA} promoter (SEQ ID NO:55), and a ribosome binding site (RBS) (SEQ ID NO:56). Element 1 controls the transcription of Element 2, a cas9 (SEQ ID NO: 44). The plasmid component sequences are shown in Table 1. The sequences of the elements present in Plasmid 12 are the same as those indicated in Example 21.

### Example 23: Plasmid 13 Construction for In Vivo Genome Editing

A representative plasmid, Plasmid 13, essentially having Plasmid Element Organization I (FIG. 9), is constructed as described in Example 21. Some Lactobacillus strains may lack endogenous recombination capacity to allow for incorporation of a provided DNA template even when that template contains sequences that are homologous to the genome. Plasmid 13 is constructed to provide a heterologous recombinase enzyme so that bacteria lacking endogenous recombination capacity can perform homologous recombination.

The in vivo gene editing plasmid is constructed using standard cloning methods. This plasmid contains the element types and structure as in Example 22 (FIG. 8) with the following differences: Element 1, a transcriptional control unit, is present at two locations in the plasmid. Transcriptional control unit #1 can be the spp-derived inducible promoter system (SEQ ID NO:54), and controls the transcription of Element 2 as described in in Example 20. Transcriptional control unit #2 can include the nis-derived inducible promoter system, which is analogous to the spp-derived inducible promoter system (SEQ ID NO:57). Transcriptional control unit #2 can contain a promoter such as the P_{nisA} promoter (SEQ ID NO:58), and a ribosome binding site (RBS) (SEQ ID NO:56). Transcriptional control unit #2 controls the transcription of Element 10. Element 10 is a gene for the expression of a heterologous recombinase operon (SEQ ID NO:59) such as LCABL 13040-50-6.

The plasmid component sequences are shown in Table 1. The sequences of the elements present in Plasmid 13 are the same as those indicated in Example 22.

### Example 24: Plasmid 14 Construction for In Vivo Genome Editing

A representative plasmid, Plasmid 14, essentially having plasmid element organization J (FIG. 10), is constructed as described in Example 23. It is not essential in all cases that the construction for genome editing in Lactobacillus strains be capable of replication in E. coli. Therefore, Plasmid 14 is constructed lacking an origin for replication in E. coli (e.g., lacking Origin #2 from example 21). Plasmid 14 is also constructed to lack an antibiotic resistance cassette for selection in E. coli such as AbR #2 from example 21. Plasmid 14, being incapable of propagating in E. coli, is constructed using a suitable host strain, such as Lactococcus lactis (e.g., Strain No. 10, Table 2) for cloning and propagation.

TABLE 2
Sequence Table

| | Sequence | |
|---|---|---|
| SEQ ID NO: | Name | DNA Sequence |
| SEQ ID NO: 1 | tetR | ATGATGTCTAGATTAGATAAAAGTAAAGTGATTAACAGCGCATTAGAGCTGCT |
| | | TAATGAGGTCGGAATCGAAGGTTTAACAACCCGTAAACTCGCCCAGAAGCTAG |
| | | GTGTAGAGCAGCCTACATTGTATTGGCATGTAAAAAATAAGCGGGCTTTGCTC |
| | | GACGCCTTAGCCATTGAGATGTTAGATAGGCACCATACTCACTTTTGCCCTTT |
| | | AGAAGGGGAAAGCTGGCAAGATTTTTTACGTAATAACGCTAAAAGTTTTAGAT |
| | | GTGCTTTACTAAGTCATCGCGATGGAGCAAAAGTACATTTAGGTACACGGCCT |
| | | ACAGAAAAACAGTATGAAACTCTCGAAAATCAATTAGCCTTTTTATGCCAACA |
| | | AGGTTTTTCACTAGAGAATGCATTATATGCACTCAGCGCTGTGGGGCATTTTA |
| | | CTTTAGGTTGCGTATTGGAAGATCAAGAGCATCAAGTCGCTAAAGAAGAAAGG |
| | | GAAACACCTACTACTGATAGTATGCCGCCATTATTACGACAAGCTATCGAATT |
| | | ATTTGATCACCAAGGTGCAGAGCCAGCCTTCTTATTCGGCCTTGAATTGATCA |
| | | TATGCGGATTAGAAAAACAACTTAAATGTGAAAGTGGGTCTTAA |
| SEQ ID NO: 2 | tet promoter | TAATTCCTAATTGCTAGCATTGTACCTAGGACTGAGCTAGCCATAAAGTTGAC |
| | | ACTCTATCGTTGATAGAGTTATTTTACCACTCCCTATCAGTGATAGAGAA |
| SEQ ID NO: 3 | RBS for Cas9 #1 | AAGAATTCAAAAGATCTAAAGAGGACTTCGGATCT |
| SEQ ID NO: 4 | Cas9 #1 | ATGGACAAGAAGTACTCTATAGGGCTTGACATAGGGACGAATAGCGTAGGGTG |
| | | GGCTGTAATAACGGACGAGTACAAGGTACCCTCTAAGAAATTCAAGGTACTTG |
| | | GGAATACGGACCGACACTCTATAAAGAAGAATCTTATAGGTGCTCTTCTTTTC |
| | | GACTCTGGGGAAACCGCTGAGGCTACGCGACTTAAGCGGACGGCTCGGCGGCG |
| | | GTACACGCGGCGAAAGAATCGAATATGTTATCTTCAGGAGATATTCTCTAATG |
| | | AGATGGCTAAGGTAGACGACTCTTTCTTCCACCGGCTTGAAGAGTCATTCCTT |
| | | GTAGAAGAGGATAAGAAGCACGAGCGACACCCCATATTCGGGAATATAGTAGA |
| | | CGAGGTAGCTTACCACGAGAAGTACCCCACGATATACCACCTTCGGAAGAAAC |
| | | TTGTAGACTCTACGGACAAGGCTGACCTTCGACTTATATATCTTGCTCTTGCT |
| | | CACATGATAAAGTTCCGAGGGCACTTCCTTATAGAGGGGGACCTTAATCCCGA |
| | | CAATTCTGACGTAGACAAGCTTTTCATACAACTTGTACAAACGTACAATCAAC |
| | | TTTTCGAGGAAAATCCCATAAATGCTTCTGGGGTAGACGCTAAGGCTATACTT |
| | | AGCGCTCGGCTTTCTAAGTCTCGGCGACTTGAAAACCTTATAGCTCAACTTCC |
| | | CGGGGAGAAGAAGAACGGGCTTTTCGGTAATCTTATTGCTCTTTCTCTTGGGC |
| | | TTACGCCCAATTTCAAGTCTAATTTCGACCTTGCTGAGGATGCTAAACTTCAA |
| | | CTTTCTAAGGACACGTACGACGACGACCTTGACAATCTTCTTGCTCAAATAGG |
| | | GGACCAATACGCTGACCTTTTTCTTGCTGCTAAGAATCTTTCAGACGCTATAC |
| | | TTCTTTCTGACATACTTCGGGTAAATACGGAGATAACGAAGGCTCCCCTTTCT |
| | | GCTAGCATGATAAAGCGGTACGACGAGCACCACCAAGACCTTACGCTTCTTAA |
| | | AGCGCTCGTACGACAACAACTTCCGGAGAAGTACAAAGAGATTTTCTTCGACC |
| | | AATCTAAGAATGGGTACGCTGGGTACATTGACGGGGGTGCTTCTCAAGAAGAG |
| | | TTCTACAAGTTCATAAAGCCCATACTTGAAAAGATGGACGGGACGGAGGAACT |
| | | TCTCGTAAAGCTTAATCGGGAGGACCTTCTTCGAAAGCAACGAACGTTCGACA |
| | | ATGGGTCTATACCCCACCAAATACACCTTGGTGAGCTTCACGCTATTCTTCGA |
| | | CGACAAGAAGATTTTTACCCGTTCCTTAAGGACAATCGAGAAAAGATAGAGAA |
| | | GATACTTACGTTCCGGATACCCTACTACGTAGGGCCGCTTGCTCGAGGTAATT |
| | | CTCGGTTCGCTTGGATGACGCGGAAGTCTGAGGAAACGATAACGCCCTGGAAT |
| | | TTCGAGGAAGTAGTAGACAAGGGGGCGTCAGCTCAATCTTTCATAGAGCGAAT |
| | | GACGAATTTCGATAAGAATCTTCCCAATGAGAAGGTACTTCCCAAGCACTCTC |
| | | TTCTTTACGAGTACTTCACGGTATATAATGAGCTTACGAAAGTAAAATACGTA |
| | | ACGGAGGGTATGCGGAAGCCCGCTTTCCTTTCTGGGGAGCAAAAAAAGGCTAT |
| | | AGTAGACCTTCTTTTCAAGACGAATCGAAAAGTAACGGTAAAGCAACTTAAAG |
| | | AGGACTACTTCAAGAAAATAGAGTGTTTCGACTCAGTAGAAATATCAGGGGTA |
| | | GAAGATCGATTCAATGCTTCACTTGGGACCTACCACGATCTTCTTAAAATTAT |
| | | AAAGGACAAGGACTTCCTTGACAACGAGGAAAATGAGGACATTCTTGAAGATA |
| | | TAGTACTTACGCTTACCCTTTTTGAGGACCGGGAGATGATAGAGGAACGACTT |
| | | AAAACGTATGCTCACCTTTTCGACGACAAAGTAATGAAGCAACTTAAGCGACG |
| | | ACGGTACACGGGGTGGGGGCGACTTTCTCGAAAGCTTATAAATGGGATACGAG |
| | | ACAAGCAATCAGGGAAGACCATACTTGATTTCCTTAAGTCAGACGGGTTCGCT |
| | | AATCGGAATTTCATGCAACTTATACACGACGACTCTCTTACGTTTAAAGAGGA |
| | | CATACAAAAAGCTCAAGTATCAGGGCAAGGGGATTCTCTTCACGAGCACATTG |
| | | CTAACCTTGCTGGGTCTCCCGCTATTAAGAAGGGGATACTTCAAACCGTAAAG |
| | | GTAGTAGACGAGCTCGTAAAAGTAATGGGGCGACACAAGCCCGAGAATATAGT |
| | | AATAGAAATGGCTCGGGAGAATCAAACGACGCAAAAGGGTCAAAAGAATTCTC |
| | | GGGAGCGGATGAAGCGAATAGAAGAGGGGATAAAAGAGCTTGGGTCTCAAATA |
| | | CTTAAAGAACACCCCGTAGAAAATACGCAACTTCAAAATGAGAAGCTTTACCT |
| | | TTACTACCTTCAAAACGGGCGAGATATGTACGTAGACCAAGAACTTGACATAA |
| | | ATCGACTTTCAGACTACGATGTAGACCATATAGTACCGCAATCTTTTCTTAAG |
| | | GACGACTCAATAGACAATAAGGTACTTACGCGGTCTGACAAGAATCGAGGGAA |
| | | GTCTGACAATGTACCCTCAGAAGAGGTAGTAAAGAAGATGAAGAATTACTGGC |
| | | GACAACTTCTTAATGCTAAGCTTATTACGCAACGGAAGTTCGACAACCTTACG |
| | | AAGGCTGAGCGGGGGGGGCTTTCTGAACTTGATAAGGCTGGGTTCATAAAGCG |
| | | GCAACTTGTAGAAACGCGACAAATAACCAAGCACGTAGCACAAATACTTGACT |
| | | CACGAATGAATACCAAGTACGACGAGAACGACAAGCTTATACGAGAAGTAAAA |
| | | GTAATAACGCTTAAGTCAAAGCTTGTATCAGATTTCCGAAAGGATTTCCAATT |
| | | TTACAAAGTACGGGAGATAAATAATTACCACCACGCTCACGACGCTTACCTTA |
| | | ATGCTGTAGTAGGTACGGCTCTTATAAAAAAGTACCCGAAGCTTGAATCTGAG |
| | | TTCGTATACGGGGACTACAAGGTATACGACGTACGAAAGATGATAGCTAAGTC |
| | | TGAGCAAGAAATAGGGAAGGCGACGGCTAAGTACTTCTTCTACTCTAATATAA |
| | | TGAATTTTTTCAAGACGGAGATTACGCTTGCTAATGGGGAGATACGAAAGCGA |
| | | CCGCTTATAGAGACCAATGGGGAAACGGGGGAGATAGTATGGGATAAGGGGCG |
| | | AGATTTTGCTACGGTACGAAAAGTACTTTCTATGCCCCAGGTAAACATAGTAA |
| | | AAAAGACGGAGGTACAAACCGGGGGGTTCTCTAAAGAGAGCATACTTCCCAAG |
| | | CGAAATTCTGATAAGCTTATAGCTCGGAAGAAGGACTGGGACCCGAAGAAGTA |
| | | CGGGGGGTTCGACTCTCCCACGGTAGCTTATAGCGTACTTGTAGTAGCTAAAG |
| | | TAGAAAAGGGGAAGTCAAAGAAACTTAAGAGCGTAAAAGAGCTTCTTGGGATA |
| | | ACGATAATGGAACGGTCTTCTTTCGAGAAGAACCCCATAGACTTTCTTGAAGC |
| | | TAAGGGGTACAAAGAAGTAAAAAAGGACCTTATAATAAAGCTTCCGAAGTACT |
| | | CACTTTTCGAGCTTGAAAATGGGCGAAAGCGGATGCTTGCTAGCGCTGGGGAA |
| | | CTTCAAAAGGGTAATGAACTTGCTCTTCCCTCAAAATATGTAAATTTCCTTTA |
| | | CCTTGCTTCTCACTATGAGAAGCTTAAGGGGTCACCCGAGGATAACGAGCAAA |
| | | AACAACTTTTTGTAGAACAACACAAGCACTACCTTGACGAGATAATAGAGCAA |
| | | ATATCTGAGTTCTCAAAGCGGGTAATACTTGCTGACGCGAACCTTGACAAAGT |
| | | ACTTTCAGCTTACAATAAGCACCGAGATAAGCCCATACGGGAGCAAGCTGAGA |
| | | ACATAATACACCTTTTTACGCTTACGAACCTTGGTGCTCCGGCTGCTTTCAAG |
| | | TACTTTGACACGACGATAGACCGAAAGCGATACACGTCTACGAAAGAGGTACT |
| | | TGACGCTACGCTTATACACCAATCTATAACGGGGCTTTACGAGACCCGAATAG |
| | | ACCTTAGCCAACTTGGTGGGGATTAA |
| SEQ ID NO: 5 | Upstream | TTTCTCTTTTGGGGCACGGATTTCCGTGCCCATTTCACAAGTTGGCTGTTATG |
| | homology #1 | CAGGAATACACGAATCATTCCGATACCACTTTTGCACTGCGTAATATCTCCTT |
| | | TCGTGTGCCCGGGCGCACGCTTTTGCATCCGCTGTCGTTAACCTTTCCTGCCG |
| | | GGAAAGTGACCGGTCTGATTGGTCACAACGGTTCTGGTAAATCCACTCTGCTC |
| | | AAAATGCTTGGCCGTCATCAGCCGCCGTCGGAAGGGGAGATTCTTCTTGATGC |
| | | CCAACCGCTGGAAAGCTGGAGCAGCAAAGCGTTTGCCCGCAAAGTGGCTTATT |
| | | TGCCGCAGCAGCTTCCTCCGGCAGAAGGGATGACCGTGCGTGAACTGGTGGCG |
| | | ATTGGTCGTTACCCGTGGCATGGCGCGCTGGGGCGCTTTGGGGCGGCAGATCG |
| | | CGAAAAAGTCGAGGAAGCTATCTCGCTGGTTGGCTTAAAACCGCTGGCGCATC |
| | | GGCTGGTCGATAGTCTCTCTGGC |
| SEQ ID NO: 6 | Downstream | CAACGCCGCTGAATCTTGTTCCGCCAGAAGATATTGCAGATATGGGCGTGGAC |
| | homology #1 | TACGACGGCAACTTTGTTTGCAGCGGTGGCATGCGTATCTTGCCGGTCTGGAC |
| | | CAGCGATCCGCAATCGCTGTGCCAGCAGAGCGAGATGCAGCAGCAGCCGTCAG |
| | | GCAATCCGTTTGATCAGTCTTCTCAGCCGCAGCAACAGCCGCAACAGCAACCT |
| | | GCTCAGCAAGAGCAGAAAGACAGCGACGGTGTAGCCGGTTGGATCAAGGATAT |
| | | GTTTGGTAGTAATTAACATCTAAGCGTGAAATACCGGATGGCGAGTTGCCATC |
| | | CGGTAAAATAACATCCCATCTAAGATATTAACCCTTTCTTTTCATCTGGTTGT |
| | | TTATTAACCCTTCAGGAACGCTCAGATTGCGTACCGCTTGCGAACCCGCCAGC |
| | | GTTTCGAATATTATCTTATCTTTATAATAATCATTCTCGTTTACGTTATCATT |
| | | CACTTTACATCAGAGATATACCA |
| SEQ ID NO: 7 | Gene insert 1 | ATGCGTAAAGGCGAAGAACTGTTCACGGGCGTAGTTCCGATTCTGGTCGAGCT |
| | | GGACGGCGATGTGAACGGTCATAAGTTTAGCGTTCGCGGTGAAGGTGAGGGCG |
| | | ACGCGACCAACGGCAAACTGACCCTGAAGTTCATCTGCACCACCGGTAAACTG |
| | | CCGGTGCCTTGGCCGACCTTGGTGACGACGTTGACGTATGGCGTGCAGTGTTT |
| | | TGCGCGTTATCCGGACCACATGAAACAACACGATTTCTTCAAATCTGCGATGC |
| | | CGGAGGGTTACGTCCAGGAGCGTACCATTTCCTTCAAGGATGATGGCTACTAC |
| | | AAAACTCGCGCAGAGGTTAAGTTTGAAGGTGACACGCTGGTCAATCGTATCGA |
| | | ATTGAAGGGTATCGACTTTAAAGAGGATGGTAACATTCTGGGCCATAAACTGG |
| | | AGTATAACTTCAACAGCCATAATGTTTACATTACGGCAGACAAGCAAAAGAAC |
| | | GGCATCAAGGCCAATTTCAAGATTCGCCACAATGTTGAGGACGGTAGCGTCCA |
| | | ACTGGCCGACCATTACCAGCAGAACACCCCAATTGGTGACGGTCCGGTTTTGC |
| | | TGCCGGATAATCACTATCTGAGCACCCAAAGCGTGCTGAGCAAAGATCCGAAC |
| | | GAAAAACGTGATCACATGGTCCTGCTGGAATTTGTGACCGCTGCGGGCATCAC |
| | | CCACGGTATGGACGAGCTGTATAAGTAATGA |
| SEQ ID NO: 8 | Gene insert 2 | ATGATTGAACAAGATGGATTGCACGCAGGTTCTCCGGCCGCTTGGGTGGAGAG |
| | | GCTATTCGGCTATGACTGGGCACAACAGACAATCGGCTGCTCTGATGCCGCCG |
| | | TGTTCCGGCTGTCAGCGCAGGGGCGCCCGGTTCTTTTTGTCAAGACCGACCTG |
| | | TCCGGTGCCCTGAATGAACTGCAGGACGAGGCAGCGCGGCTATCGTGGCTGGC |
| | | CACGACGGGCGTTCCTTGCGCAGCTGTGCTCGACGTTGTCACTGAAGCGGGAA |
| | | GGGACTGGCTGCTATTGGGCGAAGTGCCGGGGCAGGATCTCCTGTCATCTCAC |
| | | CTTGCTCCTGCCGAGAAAGTATCCATCATGGCTGATGCAATGCGGCGGCTGCA |
| | | TACGCTTGATCCGGCTACCTGCCCATTCGACCACCAAGCGAAACATCGCATCG |
| | | AGCGAGCACGTACTCGGATGGAAGCCGGTCTTGTCGATCAGGATGATCTGGAC |
| | | GAAGAGCATCAGGGGCTCGCGCCAGCCGAACTGTTCGCCAGGCTCAAGGCGCG |
| | | CATGCCCGACGGCGAGGATCTCGTCGTGACCCATGGCGATGCCTGCTTGCCGA |
| | | ATATCATGGTGGAAAATGGCCGCTTTTCTGGATTCATCGACTGTGGCCGGCTG |
| | | GGTGTGGCGGACCGCTATCAGGACATAGCGTTGGCTACCCGTGATATTGCTGA |
| | | AGAGCTTGGCGGCGAATGGGCTGACCGCTTCCTCGTGCTTTACGGTATCGCCG |
| | | CTCCCGATTCGCAGCGCATCGCCTTCTATCGCCTTCTTGACGAGTTCTTCTGA |
| SEQ ID NO: 9 | tonA sgRNA | AGACATGCCGCTAACCGCTGGTTTTAGAGCTAGAAATAGCAAGTTAAAATAAG |
| | #1 | GCTAGTCCGTTATCAACTTGAAAAAGTGGCACCGAGTCGGTGCTTTTTT |
| SEQ ID NO: 10 | p15A | TTTCCATAGGCTCCGCCCCCCTGACAAGCATCACGAAATCTGACGCTCAAATC |
| | | AGTGGTGGCGAAACCCGACAGGACTATAAAGATACCAGGCGTTTCCCCCTGGC |
| | | GGCTCCCTCGTGCGCTCTCCTGTTCCTGCCTTTCGGTTTACCGGTGTCATTCC |
| | | GCTGTTATGGCCGCGTTTGTCTCATTCCACGCCTGACACTCAGTTCCGGGTAG |
| | | GCAGTTCGCTCCAAGCTGGACTGTATGCACGAACCCCCCGTTCAGTCCGACCG |
| | | CTGCGCCTTATCCGGTAACTATCGTCTTGAGTCCAACCCGGAAAGACATGCAA |
| | | AAGCACCACTGGCAGCAGCCACTGGTAATTGATTTAGAGGAGTTAGTCTTGAA |
| | | GTCATGCGCCGGTTAAGGCTAAACTGAAAGGACAAGTTTTGGTGACTGCGCTC |
| | | CTCCAAGCCAGTTACCTCGGTTCAAAGAGTTGGTAGCTCAGAGAACCTTCGAA |
| | | AAACCGCCCTGCAAGGCGGTTTTTTCGTTTTCAGAGCAAGAGATTACGCGCAG |
| | | ACCAAAACGATCTCAA |
| SEQ ID NO: 11 | AcrIIA4 | ATGAACATCAACGATTTAATCCGTGAGATTAAGAACAAAGATTACACAGTCAA |
| | | GTTATCAGGGACAGACTCCAACTCCATCACTCAATTAATTATCCGTGTGAACA |
| | | ATGATGGTAACGAATATGTTATTAGCGAATCGGAAAATGAGAGTATTGTCGAG |
| | | AAGTTCATTAGTGCGTTTAAGAATGGCTGGAACCAAGAATACGAAGATGAAGA |
| | | AGAATTTTATAACGACATGCAAACTATCACTCTGAAGAGCGAGTTGAACTAAA |
| SEQ ID NO: 12 | RBS for | TTTTGCTACTAG |
| | AcrIIA4 | |
| SEQ ID NO: 13 | promoter for | TAGGTACTATGCTAGC |
| | AcrIIA4 | |
| SEQ ID NO: 14 | Chloramphenicol | TGATCGGCACGTAAGAGGTTCCAACTTTCACCATAATGAAATAAGATCACTAC |
| | resistance | CGGGCGTATTTTTTGAGTTATCGAGATTTTCAGGAGCTAAGGAAGCTAAAATG |
| | | GAGAAAAAAATCACTGGATATACCACCGTTGATATATCCCAATGGCATCGTAA |
| | | AGAACATTTTGAGGCATTTCAGTCAGTTGCTCAATGTACCTATAACCAGACCG |
| | | TTCAGCTGGATATTACGGCCTTTTTAAAGACCGTAAAGAAAAATAAGCACAAG |
| | | TTTTATCCGGCCTTTATTCACATTCTTGCCCGCCTGATGAATGCTCATCCGGA |
| | | ATTTCGTATGGCAATGAAAGACGGTGAGCTGGTGATATGGGATAGTGTTCACC |
| | | CTTGTTACACCGTTTTCCATGAGCAAACTGAAACGTTTTCATCGCTCTGGAGT |
| | | GAATACCACGACGATTTCCGGCAGTTTCTACACATATATTCGCAAGATGTGGC |
| | | GTGTTACGGTGAAAACCTGGCCTATTTCCCTAAAGGGTTTATTGAGAATATGT |
| | | TTTTCGTCTCAGCCAATCCCTGGGTGAGTTTCACCAGTTTTGATTTAAACGTG |
| | | GCCAATATGGACAACTTCTTCGCCCCCGTTTTCACCATGGGCAAATATTATAC |
| | | GCAAGGCGACAAGGTGCTGATGCCGCTGGCGATTCAGGTTCATCATGCCGTTT |
| | | GTGATGGCTTCCATGTCGGCAGAATGCTTAATGAATTACAACAGTACTGCGAT |
| | | GAGTGGCAGGGCGGGGCGTAA |
| SEQ ID NO: 15 | Primer 1 | TTCGTCGAGCAACAGACAAC |
| SEQ ID NO: 16 | Primer 2 | GACGCTACCGTCCTCAACATTGTG |
| SEQ ID NO: 17 | Primer 3 | AACCAGCCGACCAAACTGTATGG |
| SEQ ID NO: 18 | Primer 4 | ATGATTGAACAAGATGGATTGCACGCAG |
| SEQ ID NO: 19 | Cas9 #2 | ATGGACAAAAAATATTCTATCGGCTTAGATATCGGTACCAATTCTGTTGGTTG |
| | | GGCAGTCATTACTGACGAGTATAAAGTTCCGAGCAAAAAGTTCAAAGTCCTGG |
| | | GTAATACCGATCGCCACTCTATTAAAAAGAATCTGATCGGCGCGTTGCTGTTT |
| | | GATTCGGGTGAAACCGCAGAGGCAACCCGTCTGAAACGTACGGCTCGCCGTCG |
| | | TTATACCCGTCGTAAAAACCGCATTTGCTACTTGCAAGAAATCTTCAGCAACG |
| | | AAATGGCGAAAGTCGATGATAGCTTCTTTCACCGCCTGGAAGAGAGCTTCCTG |
| | | GTTGAGGAAGATAAGAAACACGAACGTCACCCGATCTTCGGTAACATCGTTGA |
| | | CGAAGTGGCATACCATGAAAAGTACCCAACGATTTATCATCTGCGTAAGAAAC |
| | | TGGTTGACAGCACCGATAAAGCTGATCTGCGCCTGATCTACCTGGCGCTGGCG |
| | | CACATGATTAAATTTCGCGGCCATTTCCTGATTGAGGGTGACCTGAATCCGGA |
| | | TAACTCCGATGTTGACAAGCTGTTTATCCAATTGGTTCAAACCTATAATCAGC |
| | | TGTTTGAAGAGAACCCGATCAATGCCAGCGGCGTCGACGCGAAAGCGATTCTT |
| | | AGCGCCCGTCTGTCTAAGAGCCGTCGTTTAGAAAATCTGATTGCGCAACTGCC |
| | | GGGCGAGAAGAAAAACGGCCTGTTTGGTAATTTGATTGCCCTGAGCCTGGGTC |
| | | TGACCCCAAATTTTAAAAGCAATTTCGACTTGGCAGAGGATGCTAAGCTGCAA |
| | | CTGTCCAAAGACACGTACGATGACGATCTGGACAATTTGTTGGCACAGATTGG |
| | | CGATCAGTACGCTGACCTGTTCCTGGCGGCAAAAAACTTGAGCGATGCGATTC |
| | | TGTTGAGCGACATCCTGCGCGTTAACACGGAGATCACCAAAGCCCCGCTGTCT |
| | | GCTAGCATGATCAAGCGTTATGACGAGCACCACCAGGATCTGACCCTGCTGAA |
| | | GGCATTGGTCCGTCAACAGCTGCCGGAGAAATACAAAGAAATTTTCTTCGACC |
| | | AATCCAAAAATGGTTACGCGGGCTATATTGACGGTGGCGCGAGCCAAGAGGAA |
| | | TTTTATAAGTTCATTAAACCGATTTTGGAGAAAATGGACGGTACCGAAGAACT |
| | | GCTGGTTAAACTGAACCGCGAGGATCTGCTGCGCAAGCAACGCACGTTCGATA |
| | | ACGGCTCTATCCCGCACCAGATTCACCTGGGCGAGCTGCACGCTATCCTGCGT |
| | | CGCCAAGAAGATTTCTATCCGTTCCTGAAAGACAACCGTGAAAAGATCGAGAA |
| | | AATTCTGACGTTCCGCATTCCGTACTACGTGGGTCCATTGGCACGTGGCAACA |
| | | GCCGCTTCGCTTGGATGACCCGCAAATCCGAGGAAACCATCACGCCTTGGAAC |
| | | TTTGAGGAAGTTGTGGATAAGGGTGCGTCCGCACAGAGCTTCATCGAGCGTAT |
| | | GACCAACTTCGATAAGAATCTGCCGAATGAAAAAGTGCTGCCGAAACATAGCC |
| | | TGCTGTATGAGTATTTCACCGTTTATAATGAACTGACCAAAGTTAAATACGTT |
| | | ACCGAGGGTATGCGTAAGCCAGCGTTTCTGAGCGGCGAGCAAAAAAAAGCAAT |
| | | TGTGGATCTGTTGTTCAAAACCAACCGCAAGGTAACCGTGAAACAGCTGAAAG |
| | | AAGATTACTTCAAAAAGATTGAATGTTTCGACAGCGTTGAAATCTCGGGCGTT |
| | | GAGGACCGTTTTAACGCTTCCCTGGGTACCTATCATGATTTGCTGAAAATCAT |
| | | CAAAGATAAGGACTTCTTGGATAACGAAGAAAACGAAGATATTCTGGAAGATA |
| | | TTGTCTTGACGCTGACGCTGTTTGAAGATCGCGAGATGATTGAGGAGCGTTTG |
| | | AAAACCTACGCGCATTTGTTTGACGATAAAGTGATGAAGCAACTGAAACGCCG |
| | | TCGTTACACCGGTTGGGGTCGCTTATCGCGCAAGTTGATTAACGGTATTCGCG |
| | | ACAAACAGAGCGGCAAAACTATTTTGGATTTTCTGAAATCGGACGGCTTTGCG |
| | | AACCGTAATTTCATGCAGCTGATTCATGATGATAGCTTGACCTTCAAAGAGGA |
| | | CATTCAAAAAGCCCAGGTCAGCGGTCAGGGCGACAGCCTCCACGAGCATATTG |
| | | CGAACCTGGCTGGTTCTCCGGCGATCAAGAAAGGCATCCTGCAGACCGTGAAA |
| | | GTTGTTGATGAACTGGTCAAAGTTATGGGCCGTCACAAGCCGGAAAACATTGT |
| | | GATTGAGATGGCGCGCGAGAACCAGACCACCCAAAAGGGTCAGAAGAATAGCC |
| | | GTGAGAGAATGAAACGTATCGAAGAAGGTATTAAAGAATTGGGCAGCCAGATT |
| | | CTGAAAGAGCATCCGGTCGAAAATACCCAGCTGCAAAACGAAAAACTGTACCT |
| | | GTACTACTTGCAAAATGGTCGTGATATGTACGTGGATCAGGAACTGGACATCA |
| | | ACCGCCTGAGCGACTATGATGTTGATCACATCGTGCCGCAATCCTTTCTGAAG |
| | | GATGACAGCATCGACAATAAAGTTTTGACTCGCTCGGATAAGAATCGTGGTAA |
| | | GTCCGACAACGTGCCGAGCGAAGAAGTCGTGAAGAAAATGAAGAATTATTGGC |
| | | GTCAACTGCTTAATGCCAAACTGATCACCCAACGTAAGTTTGACAATCTGACG |
| | | AAAGCCGAGCGCGGTGGTCTGAGCGAGCTGGATAAGGCCGGTTTTATCAAGCG |
| | | TCAGCTCGTCGAAACGCGTCAGATCACCAAACATGTCGCACAAATCTTAGATT |
| | | CCCGCATGAATACGAAATACGACGAGAACGACAAGCTGATCCGTGAAGTTAAA |
| | | GTGATTACCCTGAAATCTAAACTGGTGAGCGATTTCCGTAAAGACTTCCAGTT |
| | | TTACAAGGTTCGCGAGATCAACAATTATCACCATGCCCACGACGCTTACCTGA |
| | | ATGCAGTGGTTGGTACCGCACTGATCAAGAAATATCCGAAGCTGGAGAGCGAG |
| | | TTTGTGTACGGTGATTACAAAGTCTACGATGTCCGTAAGATGATCGCAAAATC |
| | | TGAACAAGAGATCGGTAAAGCGACGGCGAAGTACTTTTTCTATAGCAACATTA |
| | | TGAACTTTTTTAAAACCGAAATCACCCTGGCCAACGGCGAGATCCGCAAGCGT |
| | | CCGCTGATCGAAACGAACGGCGAAACGGGCGAGATTGTGTGGGACAAGGGTCG |
| | | CGACTTCGCTACTGTCCGTAAAGTGCTGAGCATGCCTCAGGTGAATATCGTCA |
| | | AAAAGACCGAAGTTCAGACCGGTGGTTTCAGCAAAGAGAGCATCTTGCCGAAG |
| | | CGTAACAGCGACAAACTGATTGCCCGTAAAAAAGATTGGGACCCGAAGAAATA |
| | | CGGCGGCTTCGATTCGCCGACCGTTGCATATTCAGTTCTGGTCGTGGCAAAAG |
| | | TTGAAAAGGGCAAGTCCAAAAAGTTGAAGTCCGTTAAAGAGCTGCTGGGTATT |
| | | ACTATTATGGAACGCAGCTCCTTCGAGAAGAATCCGATTGACTTCCTGGAAGC |
| | | GAAGGGCTATAAAGAGGTTAAGAAAGATCTGATCATCAAGCTGCCGAAGTACA |
| | | GCCTGTTTGAGCTGGAAAATGGCCGTAAACGTATGCTGGCGTCTGCAGGCGAA |
| | | CTGCAAAAGGGTAACGAACTGGCGCTGCCGAGCAAATATGTCAATTTTCTCTA |
| | | TCTGGCCAGCCACTACGAGAAACTGAAGGGTTCTCCTGAAGATAACGAACAGA |
| | | AACAGCTGTTTGTCGAGCAGCATAAACACTATCTGGACGAAATCATCGAACAG |
| | | ATCAGCGAGTTCTCTAAGCGTGTCATCCTGGCTGACGCGAATCTGGACAAAGT |
| | | GCTGTCCGCATATAACAAGCACCGTGACAAGCCGATCCGTGAACAGGCTGAAA |
| | | ACATCATCCACCTGTTTACCCTGACGAACTTGGGTGCCCCGGCGGCGTTCAAA |
| | | TACTTTGACACGACCATCGATCGTAAACGTTACACGAGCACTAAAGAGGTCCT |
| | | GGACGCGACGCTGATTCACCAAAGCATCACGGGCCTGTACGAAACTCGTATCG |
| | | ACCTGTCCCAACTGGGTGGCGACTAA |
| SEQ ID NO: 20 | nCas9 | ATGGACAAAAAATATTCTATCGGCTTAGCCATCGGTACCAATTCTGTTGGTTG |
| | | GGCAGTCATTACTGACGAGTATAAAGTTCCGAGCAAAAAGTTCAAAGTCCTGG |
| | | GTAATACCGATCGCCACTCTATTAAAAAGAATCTGATCGGCGCGTTGCTGTTT |
| | | GATTCGGGTGAAACCGCAGAGGCAACCCGTCTGAAACGTACGGCTCGCCGTCG |
| | | TTATACCCGTCGTAAAAACCGCATTTGCTACTTGCAAGAAATCTTCAGCAACG |
| | | AAATGGCGAAAGTCGATGATAGCTTCTTTCACCGCCTGGAAGAGAGCTTCCTG |
| | | GTTGAGGAAGATAAGAAACACGAACGTCACCCGATCTTCGGTAACATCGTTGA |
| | | CGAAGTGGCATACCATGAAAAGTACCCAACGATTTATCATCTGCGTAAGAAAC |
| | | TGGTTGACAGCACCGATAAAGCTGATCTGCGCCTGATCTACCTGGCGCTGGCG |
| | | CACATGATTAAATTTCGCGGCCATTTCCTGATTGAGGGTGACCTGAATCCGGA |
| | | TAACTCCGATGTTGACAAGCTGTTTATCCAATTGGTTCAAACCTATAATCAGC |
| | | TGTTTGAAGAGAACCCGATCAATGCCAGCGGCGTCGACGCGAAAGCGATTCTT |
| | | AGCGCCCGTCTGTCTAAGAGCCGTCGTTTAGAAAATCTGATTGCGCAACTGCC |
| | | GGGCGAGAAGAAAAACGGCCTGTTTGGTAATTTGATTGCCCTGAGCCTGGGTC |
| | | TGACCCCAAATTTTAAAAGCAATTTCGACTTGGCAGAGGATGCTAAGCTGCAA |
| | | CTGTCCAAAGACACGTACGATGACGATCTGGACAATTTGTTGGCACAGATTGG |
| | | CGATCAGTACGCTGACCTGTTCCTGGCGGCAAAAAACTTGAGCGATGCGATTC |
| | | TGTTGAGCGACATCCTGCGCGTTAACACGGAGATCACCAAAGCCCCGCTGTCT |
| | | GCTAGCATGATCAAGCGTTATGACGAGCACCACCAGGATCTGACCCTGCTGAA |
| | | GGCATTGGTCCGTCAACAGCTGCCGGAGAAATACAAAGAAATTTTCTTCGACC |
| | | AATCCAAAAATGGTTACGCGGGCTATATTGACGGTGGCGCGAGCCAAGAGGAA |
| | | TTTTATAAGTTCATTAAACCGATTTTGGAGAAAATGGACGGTACCGAAGAACT |
| | | GCTGGTTAAACTGAACCGCGAGGATCTGCTGCGCAAGCAACGCACGTTCGATA |
| | | ACGGCTCTATCCCGCACCAGATTCACCTGGGCGAGCTGCACGCTATCCTGCGT |
| | | CGCCAAGAAGATTTCTATCCGTTCCTGAAAGACAACCGTGAAAAGATCGAGAA |
| | | AATTCTGACGTTCCGCATTCCGTACTACGTGGGTCCATTGGCACGTGGCAACA |
| | | GCCGCTTCGCTTGGATGACCCGCAAATCCGAGGAAACCATCACGCCTTGGAAC |
| | | TTTGAGGAAGTTGTGGATAAGGGTGCGTCCGCACAGAGCTTCATCGAGCGTAT |
| | | GACCAACTTCGATAAGAATCTGCCGAATGAAAAAGTGCTGCCGAAACATAGCC |
| | | TGCTGTATGAGTATTTCACCGTTTATAATGAACTGACCAAAGTTAAATACGTT |
| | | ACCGAGGGTATGCGTAAGCCAGCGTTTCTGAGCGGCGAGCAAAAAAAAGCAAT |
| | | TGTGGATCTGTTGTTCAAAACCAACCGCAAGGTAACCGTGAAACAGCTGAAAG |
| | | AAGATTACTTCAAAAAGATTGAATGTTTCGACAGCGTTGAAATCTCGGGCGTT |
| | | GAGGACCGTTTTAACGCTTCCCTGGGTACCTATCATGATTTGCTGAAAATCAT |
| | | CAAAGATAAGGACTTCTTGGATAACGAAGAAAACGAAGATATTCTGGAAGATA |
| | | TTGTCTTGACGCTGACGCTGTTTGAAGATCGCGAGATGATTGAGGAGCGTTTG |
| | | AAAACCTACGCGCATTTGTTTGACGATAAAGTGATGAAGCAACTGAAACGCCG |
| | | TCGTTACACCGGTTGGGGTCGCTTATCGCGCAAGTTGATTAACGGTATTCGCG |
| | | ACAAACAGAGCGGCAAAACTATTTTGGATTTTCTGAAATCGGACGGCTTTGCG |
| | | AACCGTAATTTCATGCAGCTGATTCATGATGATAGCTTGACCTTCAAAGAGGA |
| | | CATTCAAAAAGCCCAGGTCAGCGGTCAGGGCGACAGCCTCCACGAGCATATTG |
| | | CGAACCTGGCTGGTTCTCCGGCGATCAAGAAAGGCATCCTGCAGACCGTGAAA |
| | | GTTGTTGATGAACTGGTCAAAGTTATGGGCCGTCACAAGCCGGAAAACATTGT |
| | | GATTGAGATGGCGCGCGAGAACCAGACCACCCAAAAGGGTCAGAAGAATAGCC |
| | | GTGAGAGAATGAAACGTATCGAAGAAGGTATTAAAGAATTGGGCAGCCAGATT |
| | | CTGAAAGAGCATCCGGTCGAAAATACCCAGCTGCAAAACGAAAAACTGTACCT |
| | | GTACTACTTGCAAAATGGTCGTGATATGTACGTGGATCAGGAACTGGACATCA |
| | | ACCGCCTGAGCGACTATGATGTTGATCACATCGTGCCGCAATCCTTTCTGAAG |
| | | GATGACAGCATCGACAATAAAGTTTTGACTCGCTCGGATAAGAATCGTGGTAA |
| | | GTCCGACAACGTGCCGAGCGAAGAAGTCGTGAAGAAAATGAAGAATTATTGGC |
| | | GTCAACTGCTTAATGCCAAACTGATCACCCAACGTAAGTTTGACAATCTGACG |
| | | AAAGCCGAGCGCGGTGGTCTGAGCGAGCTGGATAAGGCCGGTTTTATCAAGCG |
| | | TCAGCTCGTCGAAACGCGTCAGATCACCAAACATGTCGCACAAATCTTAGATT |
| | | CCCGCATGAATACGAAATACGACGAGAACGACAAGCTGATCCGTGAAGTTAAA |
| | | GTGATTACCCTGAAATCTAAACTGGTGAGCGATTTCCGTAAAGACTTCCAGTT |
| | | TTACAAGGTTCGCGAGATCAACAATTATCACCATGCCCACGACGCTTACCTGA |
| | | ATGCAGTGGTTGGTACCGCACTGATCAAGAAATATCCGAAGCTGGAGAGCGAG |
| | | TTTGTGTACGGTGATTACAAAGTCTACGATGTCCGTAAGATGATCGCAAAATC |
| | | TGAACAAGAGATCGGTAAAGCGACGGCGAAGTACTTTTTCTATAGCAACATTA |
| | | TGAACTTTTTTAAAACCGAAATCACCCTGGCCAACGGCGAGATCCGCAAGCGT |
| | | CCGCTGATCGAAACGAACGGCGAAACGGGCGAGATTGTGTGGGACAAGGGTCG |
| | | CGACTTCGCTACTGTCCGTAAAGTGCTGAGCATGCCTCAGGTGAATATCGTCA |
| | | AAAAGACCGAAGTTCAGACCGGTGGTTTCAGCAAAGAGAGCATCTTGCCGAAG |
| | | CGTAACAGCGACAAACTGATTGCCCGTAAAAAAGATTGGGACCCGAAGAAATA |
| | | CGGCGGCTTCGATTCGCCGACCGTTGCATATTCAGTTCTGGTCGTGGCAAAAG |
| | | TTGAAAAGGGCAAGTCCAAAAAGTTGAAGTCCGTTAAAGAGCTGCTGGGTATT |
| | | ACTATTATGGAACGCAGCTCCTTCGAGAAGAATCCGATTGACTTCCTGGAAGC |
| | | GAAGGGCTATAAAGAGGTTAAGAAAGATCTGATCATCAAGCTGCCGAAGTACA |
| | | GCCTGTTTGAGCTGGAAAATGGCCGTAAACGTATGCTGGCGTCTGCAGGCGAA |
| | | CTGCAAAAGGGTAACGAACTGGCGCTGCCGAGCAAATATGTCAATTTTCTCTA |
| | | TCTGGCCAGCCACTACGAGAAACTGAAGGGTTCTCCTGAAGATAACGAACAGA |
| | | AACAGCTGTTTGTCGAGCAGCATAAACACTATCTGGACGAAATCATCGAACAG |
| | | ATCAGCGAGTTCTCTAAGCGTGTCATCCTGGCTGACGCGAATCTGGACAAAGT |
| | | GCTGTCCGCATATAACAAGCACCGTGACAAGCCGATCCGTGAACAGGCTGAAA |
| | | ACATCATCCACCTGTTTACCCTGACGAACTTGGGTGCCCCGGCGGCGTTCAAA |
| | | TACTTTGACACGACCATCGATCGTAAACGTTACACGAGCACTAAAGAGGTCCT |
| | | GGACGCGACGCTGATTCACCAAAGCATCACGGGCCTGTACGAAACTCGTATCG |
| | | ACCTGTCCCAACTGGGTGGCGACTAA |
| SEQ ID NO: 21 | tonA sgRNA | AGGTAGATTATCAGCTTTCGCTAAGGATGATTTCTGGAATTCTTCCCTATCAG |
| | pair #1 | TGATAGAGATTGACATCCCTATCAGTGATAGAGATACTGAGCACCGCAGTTGT |
| | | AGTAGCCACAGGTTTTAGAGCTAGAAATAGCAAGTTAAAATAAGGCTAGTCCG |
| | | TTATCAACTTGAAAAAGTGGCACCGAGTCGGTGCTTTTTTCCACAGATTATCA |
| | | GCTTTCGCTAAGGATGATTTCTGGAATTCTTCCCTATCAGTGATAGAGATTGA |
| | | CATCCCTATCAGTGATAGAGATACTGAGCACTTCTTGCGGCGCAGGTGCAGGT |
| | | TTTAGAGCTAGAAATAGCAAGTTAAAATAAGGCTAGTCCGTTATCAACTTGAA |
| | | AAAGTGGCACCGAGTCGGTGCTTTTTT |
| SEQ ID NO: 22 | tonA sgRNA | AGGTAGATTATCAGCTTTCGCTAAGGATGATTTCTGGAATTCTTCCCTATCAG |
| | pair #2 | TGATAGAGATTGACATCCCTATCAGTGATAGAGATACTGAGCACCGTTATGAT |
| | | CTGGCGCGAGTGTTTTAGAGCTAGAAATAGCAAGTTAAAATAAGGCTAGTCCG |
| | | TTATCAACTTGAAAAAGTGGCACCGAGTCGGTGCTTTTTTCCACAGATTATCA |
| | | GCTTTCGCTAAGGATGATTTCTGGAATTCTTCCCTATCAGTGATAGAGATTGA |
| | | CATCCCTATCAGTGATAGAGATACTGAGCACGCCATAAGTGTTAAAGCAGCGT |
| | | TTTAGAGCTAGAAATAGCAAGTTAAAATAAGGCTAGTCCGTTATCAACTTGAA |
| | | AAAGTGGCACCGAGTCGGTGCTTTTTT |
| SEQ ID NO: 23 | tonA sgRNA | AGGTAGATTATCAGCTTTCGCTAAGGATGATTTCTGGAATTCTTCCCTATCAG |
| | pair #3 | TGATAGAGATTGACATCCCTATCAGTGATAGAGATACTGAGCACAGACATGCC |
| | | GCTAACCGCTGGTTTTAGAGCTAGAAATAGCAAGTTAAAATAAGGCTAGTCCG |
| | | TTATCAACTTGAAAAAGTGGCACCGAGTCGGTGCTTTTTTCCACAGATTATCA |
| | | GCTTTCGCTAAGGATGATTTCTGGAATTCTTCCCTATCAGTGATAGAGATTGA |
| | | CATCCCTATCAGTGATAGAGATACTGAGCACCGTTATGATCTGGCGCGAGTGT |
| | | TTTAGAGCTAGAAATAGCAAGTTAAAATAAGGCTAGTCCGTTATCAACTTGAA |
| | | AAAGTGGCACCGAGTCGGTGCTTTTTT |
| SEQ ID NO: 24 | Insert DNA | CATGGTCCTGCTGGAATTTGTG |
| | probe | |
| SEQ ID NO: 25 | Target DNA | CGGGACGACCGATAAACGTGA |
| | probe | |
| SEQ ID NO: 26 | dCas9 | ATGGACAAAAAATATTCTATCGGCTTAGCCATCGGTACCAATTCTGTTGGTTG |
| | | GGCAGTCATTACTGACGAGTATAAAGTTCCGAGCAAAAAGTTCAAAGTCCTGG |
| | | GTAATACCGATCGCCACTCTATTAAAAAGAATCTGATCGGCGCGTTGCTGTTT |
| | | GATTCGGGTGAAACCGCAGAGGCAACCCGTCTGAAACGTACGGCTCGCCGTCG |
| | | TTATACCCGTCGTAAAAACCGCATTTGCTACTTGCAAGAAATCTTCAGCAACG |
| | | AAATGGCGAAAGTCGATGATAGCTTCTTTCACCGCCTGGAAGAGAGCTTCCTG |
| | | GTTGAGGAAGATAAGAAACACGAACGTCACCCGATCTTCGGTAACATCGTTGA |
| | | CGAAGTGGCATACCATGAAAAGTACCCAACGATTTATCATCTGCGTAAGAAAC |
| | | TGGTTGACAGCACCGATAAAGCTGATCTGCGCCTGATCTACCTGGCGCTGGCG |
| | | CACATGATTAAATTTCGCGGCCATTTCCTGATTGAGGGTGACCTGAATCCGGA |
| | | TAACTCCGATGTTGACAAGCTGTTTATCCAATTGGTTCAAACCTATAATCAGC |
| | | TGTTTGAAGAGAACCCGATCAATGCCAGCGGCGTCGACGCGAAAGCGATTCTT |
| | | AGCGCCCGTCTGTCTAAGAGCCGTCGTTTAGAAAATCTGATTGCGCAACTGCC |
| | | GGGCGAGAAGAAAAACGGCCTGTTTGGTAATTTGATTGCCCTGAGCCTGGGTC |
| | | TGACCCCAAATTTTAAAAGCAATTTCGACTTGGCAGAGGATGCTAAGCTGCAA |
| | | CTGTCCAAAGACACGTACGATGACGATCTGGACAATTTGTTGGCACAGATTGG |
| | | CGATCAGTACGCTGACCTGTTCCTGGCGGCAAAAAACTTGAGCGATGCGATTC |
| | | TGTTGAGCGACATCCTGCGCGTTAACACGGAGATCACCAAAGCCCCGCTGTCT |
| | | GCTAGCATGATCAAGCGTTATGACGAGCACCACCAGGATCTGACCCTGCTGAA |
| | | GGCATTGGTCCGTCAACAGCTGCCGGAGAAATACAAAGAAATTTTCTTCGACC |
| | | AATCCAAAAATGGTTACGCGGGCTATATTGACGGTGGCGCGAGCCAAGAGGAA |
| | | TTTTATAAGTTCATTAAACCGATTTTGGAGAAAATGGACGGTACCGAAGAACT |
| | | GCTGGTTAAACTGAACCGCGAGGATCTGCTGCGCAAGCAACGCACGTTCGATA |
| | | ACGGCTCTATCCCGCACCAGATTCACCTGGGCGAGCTGCACGCTATCCTGCGT |
| | | CGCCAAGAAGATTTCTATCCGTTCCTGAAAGACAACCGTGAAAAGATCGAGAA |
| | | AATTCTGACGTTCCGCATTCCGTACTACGTGGGTCCATTGGCACGTGGCAACA |
| | | GCCGCTTCGCTTGGATGACCCGCAAATCCGAGGAAACCATCACGCCTTGGAAC |
| | | TTTGAGGAAGTTGTGGATAAGGGTGCGTCCGCACAGAGCTTCATCGAGCGTAT |
| | | GACCAACTTCGATAAGAATCTGCCGAATGAAAAAGTGCTGCCGAAACATAGCC |
| | | TGCTGTATGAGTATTTCACCGTTTATAATGAACTGACCAAAGTTAAATACGTT |
| | | ACCGAGGGTATGCGTAAGCCAGCGTTTCTGAGCGGCGAGCAAAAAAAAGCAAT |
| | | TGTGGATCTGTTGTTCAAAACCAACCGCAAGGTAACCGTGAAACAGCTGAAAG |
| | | AAGATTACTTCAAAAAGATTGAATGTTTCGACAGCGTTGAAATCTCGGGCGTT |
| | | GAGGACCGTTTTAACGCTTCCCTGGGTACCTATCATGATTTGCTGAAAATCAT |
| | | CAAAGATAAGGACTTCTTGGATAACGAAGAAAACGAAGATATTCTGGAAGATA |
| | | TTGTCTTGACGCTGACGCTGTTTGAAGATCGCGAGATGATTGAGGAGCGTTTG |
| | | AAAACCTACGCGCATTTGTTTGACGATAAAGTGATGAAGCAACTGAAACGCCG |
| | | TCGTTACACCGGTTGGGGTCGCTTATCGCGCAAGTTGATTAACGGTATTCGCG |
| | | ACAAACAGAGCGGCAAAACTATTTTGGATTTTCTGAAATCGGACGGCTTTGCG |
| | | AACCGTAATTTCATGCAGCTGATTCATGATGATAGCTTGACCTTCAAAGAGGA |
| | | CATTCAAAAAGCCCAGGTCAGCGGTCAGGGCGACAGCCTCCACGAGCATATTG |
| | | CGAACCTGGCTGGTTCTCCGGCGATCAAGAAAGGCATCCTGCAGACCGTGAAA |
| | | GTTGTTGATGAACTGGTCAAAGTTATGGGCCGTCACAAGCCGGAAAACATTGT |
| | | GATTGAGATGGCGCGCGAGAACCAGACCACCCAAAAGGGTCAGAAGAATAGCC |
| | | GTGAGAGAATGAAACGTATCGAAGAAGGTATTAAAGAATTGGGCAGCCAGATT |
| | | CTGAAAGAGCATCCGGTCGAAAATACCCAGCTGCAAAACGAAAAACTGTACCT |
| | | GTACTACTTGCAAAATGGTCGTGATATGTACGTGGATCAGGAACTGGACATCA |
| | | ACCGCCTGAGCGACTATGATGTTGATGCCATCGTGCCGCAATCCTTTCTGAAG |
| | | GATGACAGCATCGACAATAAAGTTTTGACTCGCTCGGATAAGAATCGTGGTAA |
| | | GTCCGACAACGTGCCGAGCGAAGAAGTCGTGAAGAAAATGAAGAATTATTGGC |
| | | GTCAACTGCTTAATGCCAAACTGATCACCCAACGTAAGTTTGACAATCTGACG |
| | | AAAGCCGAGCGCGGTGGTCTGAGCGAGCTGGATAAGGCCGGTTTTATCAAGCG |
| | | TCAGCTCGTCGAAACGCGTCAGATCACCAAACATGTCGCACAAATCTTAGATT |
| | | CCCGCATGAATACGAAATACGACGAGAACGACAAGCTGATCCGTGAAGTTAAA |
| | | GTGATTACCCTGAAATCTAAACTGGTGAGCGATTTCCGTAAAGACTTCCAGTT |
| | | TTACAAGGTTCGCGAGATCAACAATTATCACCATGCCCACGACGCTTACCTGA |
| | | ATGCAGTGGTTGGTACCGCACTGATCAAGAAATATCCGAAGCTGGAGAGCGAG |
| | | TTTGTGTACGGTGATTACAAAGTCTACGATGTCCGTAAGATGATCGCAAAATC |
| | | TGAACAAGAGATCGGTAAAGCGACGGCGAAGTACTTTTTCTATAGCAACATTA |
| | | TGAACTTTTTTAAAACCGAAATCACCCTGGCCAACGGCGAGATCCGCAAGCGT |
| | | CCGCTGATCGAAACGAACGGCGAAACGGGCGAGATTGTGTGGGACAAGGGTCG |
| | | CGACTTCGCTACTGTCCGTAAAGTGCTGAGCATGCCTCAGGTGAATATCGTCA |
| | | AAAAGACCGAAGTTCAGACCGGTGGTTTCAGCAAAGAGAGCATCTTGCCGAAG |
| | | CGTAACAGCGACAAACTGATTGCCCGTAAAAAAGATTGGGACCCGAAGAAATA |
| | | CGGCGGCTTCGATTCGCCGACCGTTGCATATTCAGTTCTGGTCGTGGCAAAAG |
| | | TTGAAAAGGGCAAGTCCAAAAAGTTGAAGTCCGTTAAAGAGCTGCTGGGTATT |
| | | ACTATTATGGAACGCAGCTCCTTCGAGAAGAATCCGATTGACTTCCTGGAAGC |
| | | GAAGGGCTATAAAGAGGTTAAGAAAGATCTGATCATCAAGCTGCCGAAGTACA |
| | | GCCTGTTTGAGCTGGAAAATGGCCGTAAACGTATGCTGGCGTCTGCAGGCGAA |
| | | CTGCAAAAGGGTAACGAACTGGCGCTGCCGAGCAAATATGTCAATTTTCTCTA |
| | | TCTGGCCAGCCACTACGAGAAACTGAAGGGTTCTCCTGAAGATAACGAACAGA |
| | | AACAGCTGTTTGTCGAGCAGCATAAACACTATCTGGACGAAATCATCGAACAG |
| | | ATCAGCGAGTTCTCTAAGCGTGTCATCCTGGCTGACGCGAATCTGGACAAAGT |
| | | GCTGTCCGCATATAACAAGCACCGTGACAAGCCGATCCGTGAACAGGCTGAAA |
| | | ACATCATCCACCTGTTTACCCTGACGAACTTGGGTGCCCCGGCGGCGTTCAAA |
| | | TACTTTGACACGACCATCGATCGTAAACGTTACACGAGCACTAAAGAGGTCCT |
| | | GGACGCGACGCTGATTCACCAAAGCATCACGGGCCTGTACGAAACTCGTATCG |
| | | ACCTGTCCCAACTGGGTGGCGACTAA |
| SEQ ID NO: 27 | gfp, lacZ, | AGGTAGATTATCAGCTTTCGCTAAGGATGATTTCTGGAATTCTTCCCTATCAG |
| | gusA, flhC | TGATAGAGATTGACATCCCTATCAGTGATAGAGATACTGAGCACAAAGGCGAA |
| | sgRNA | GAACTGTTCACGTTTTAGAGCTAGAAATAGCAAGTTAAAATAAGGCTAGTCCG |
| | | TTATCAACTTGAAAAAGTGGCACCGAGTCGGTGCTTTTTTCCACAGATTATCA |
| | | GCTTTCGCTAAGGATGATTTCTGGAATTCTTCCCTATCAGTGATAGAGATTGA |
| | | CATCCCTATCAGTGATAGAGATACTGAGCACAGCGGATAACAATTTCACACGT |
| | | TTTAGAGCTAGAAATAGCAAGTTAAAATAAGGCTAGTCCGTTATCAACTTGAA |
| | | AAAGTGGCACCGAGTCGGTGCTTTTTTGGTAAGATTATCAGCTTTCGCTAAGG |
| | | ATGATTTCTGGAATTCTTCCCTATCAGTGATAGAGATTGACATCCCTATCAGT |
| | | GATAGAGATACTGAGCACCGGCCTGTGGGCATTCAGTCGTTTTAGAGCTAGAA |
| | | ATAGCAAGTTAAAATAAGGCTAGTCCGTTATCAACTTGAAAAAGTGGCACCGA |
| | | GTCGGTGCTTTTTTCCGAAGATTATCAGCTTTCGCTAAGGATGATTTCTGGAA |
| | | TTCTTCCCTATCAGTGATAGAGATTGACATCCCTATCAGTGATAGAGATACTG |
| | | AGCACGCATCTGCAAACGAGCGCCCGTTTTAGAGCTAGAAATAGCAAGTTAAA |
| | | ATAAGGCTAGTCCGTTATCAACTTGAAAAAGTGGCACCGAGTCGGTGCTTTTT |
| | | T |
| SEQ ID NO: 28 | tetR #2 | ATGTCGCGCCTGGACAAATCCAAGGTGATCAACAGCGCACTGGAACTGCTGAA |
| | | CGAAGTGGGCATCGAAGGACTGACCACGCGCAAACTGGCCCAGAAGCTGGGCG |
| | | TGGAACAGCCGACACTGTACTGGCATGTGAAAAACAAGCGCGCACTGCTGGAC |
| | | GCCCTGGCAATCGAAATGCTGGACCGCCATCACACCCACTTCTGCCCGCTGGA |
| | | AGGAGAATCCTGGCAGGACTTCCTGCGCAACAACGCAAAATCGTTCCGCTGCG |
| | | CCCTGCTGTCCCATAGAGACGGCGCAAAAGTGCACCTGGGAACGCGCCCGACA |
| | | GAAAAGCAGTACGAAACGCTGGAAAACCAGCTGGCCTTCCTGTGCCAGCAGGG |
| | | CTTCAGCCTGGAAAACGCCCTGTATGCACTGTCGGCCGTGGGCCATTTCACCC |
| | | TGGGATGCGTGCTGGAAGACCAGGAACACCAGGTGGCAAAGGAAGAACGCGAA |
| | | ACGCCGACGACGGACAGCATGCCGCCGCTGCTGAGACAGGCAATCGAACTGTT |
| | | CGACCATCAGGGCGCAGAACCGGCCTTCCTGTTCGGCCTGGAACTGATCATCT |
| | | GCGGACTGGAAAAACAGCTGAAGTGCGAATCCGGAAGCTAA |
| SEQ ID NO: 29 | RBS for tet | CGCATTTTAAAATAAAATAAATTATTTATGATATTAAACGAAT |
| SEQ ID NO: 30 | P2 promoter | AAGAAAAGGCGTTTTGTTTTTCTTCTTTACCTTCTTTCCCTTTCGCTAAGAGA |
| | for tet | GTCTGAGAAACGATAGAAAAAGAAAAGCGAAAAAACTTCCGAAAACATTTGGT |
| | | AGTTAAAATAAAACCTCTTACCTTTGCACCCG |
| SEQ ID NO: 31 | P1TDP- | TTTTGCACCCGCTTTCCAAGAGAAGAAAGCCTTGTTAAATTGACTTAGTGTAA |
| | GH023 | AAGCGCAGTACTGCTTGACCATAAGAACAAAAAAATCTCTATCACTGATAGGG |
| | promoter for | ATAAAGTTTGGAAGATAAAGCTAAAAGTTCTTATCTTTGCAGTCTCCCTATCA |
| | Cas9 #3 | GTGATAGAGACGAAATAAAGACATATAAAAGAAAAGACACC |
| SEQ ID NO: 32 | Cas9 #3 | ATGGACAAAAAGTATTCGATCGGCCTGGACATCGGAACAAACTCCGTGGGCTG |
| | | GGCCGTGATCACCGACGAATACAAGGTGCCGTCGAAAAAGTTCAAAGTGCTGG |
| | | GAAACACGGACCGCCATTCCATCAAAAAGAACCTGATCGGCGCCCTGCTGTTC |
| | | GACAGCGGAGAAACGGCCGAAGCAACGAGACTGAAACGCACCGCACGCCGCCG |
| | | CTACACACGTCGCAAAAACCGCATCTGCTACCTGCAGGAAATCTTCTCCAACG |
| | | AAATGGCAAAAGTGGACGACAGCTTCTTCCACCGCCTGGAAGAATCGTTCCTG |
| | | GTGGAAGAAGACAAAAAGCATGAACGCCACCCGATCTTCGGCAACATCGTGGA |
| | | CGAAGTGGCCTATCATGAAAAGTACCCGACGATCTATCATCTGCGCAAAAAAC |
| | | TGGTGGACTCCACAGACAAGGCAGACCTGCGCCTGATCTATCTGGCCCTGGCA |
| | | CACATGATCAAATTCCGCGGCCACTTCCTGATCGAAGGAGACCTGAACCCGGA |
| | | CAACAGCGACGTGGACAAACTGTTCATCCAGCTGGTGCAGACATACAACCAGC |
| | | TGTTCGAAGAAAACCCGATCAACGCCAGCGGCGTGGACGCCAAGGCAATCCTG |
| | | TCCGCAAGACTGTCGAAATCCCGCCGCCTGGAAAACCTGATCGCCCAGCTGCC |
| | | GGGCGAAAAGAAAAACGGCCTGTTCGGAAACCTGATCGCACTGTCCCTGGGAC |
| | | TGACCCCGAACTTCAAAAGCAACTTCGACCTGGCCGAAGACGCAAAGCTGCAG |
| | | CTGTCCAAAGACACGTATGACGACGACCTGGACAACCTGCTGGCCCAGATCGG |
| | | AGACCAGTACGCAGACCTGTTCCTGGCCGCAAAGAACCTGAGCGACGCCATCC |
| | | TGCTGTCGGACATCCTGCGCGTGAACACCGAAATCACGAAGGCCCCGCTGAGC |
| | | GCCTCCATGATCAAACGCTATGACGAACATCACCAGGACCTGACCCTGCTGAA |
| | | AGCACTGGTGCGCCAGCAGCTGCCGGAAAAATACAAGGAAATCTTCTTCGACC |
| | | AGTCGAAGAACGGCTACGCCGGATATATCGACGGCGGAGCATCCCAGGAAGAA |
| | | TTTTACAAATTCATCAAGCCGATCCTGGAAAAAATGGACGGCACAGAAGAACT |
| | | GCTGGTGAAGCTGAACCGCGAAGACCTGCTGCGCAAACAGCGCACCTTCGACA |
| | | ACGGCAGCATCCCGCATCAGATCCACCTGGGAGAACTGCATGCCATCCTGCGT |
| | | CGCCAGGAAGACTTCTACCCGTTCCTGAAGGACAACCGCGAAAAAATCGAAAA |
| | | GATCCTGACATTCCGCATCCCGTACTATGTGGGACCGCTGGCCCGCGGAAACT |
| | | CGCGCTTCGCATGGATGACCCGCAAGTCCGAAGAAACGATCACACCGTGGAAC |
| | | TTCGAAGAAGTGGTGGACAAAGGAGCCTCCGCACAGAGCTTCATCGAACGCAT |
| | | GACCAACTTCGACAAGAACCTGCCGAACGAAAAGGTGCTGCCGAAACACTCGC |
| | | TGCTGTACGAATATTTCACCGTGTATAACGAACTGACGAAAGTGAAGTACGTG |
| | | ACAGAAGGCATGCGCAAACCGGCCTTCCTGTCCGGAGAACAGAAAAAGGCAAT |
| | | CGTGGACCTGCTGTTCAAGACCAACCGCAAAGTGACGGTGAAACAGCTGAAGG |
| | | AAGACTATTTCAAAAAGATCGAATGCTTCGACTCCGTGGAAATCAGCGGCGTG |
| | | GAAGACCGCTTCAACGCCTCCCTGGGAACGTACCATGACCTGCTGAAAATCAT |
| | | CAAAGACAAGGACTTCCTGGACAACGAAGAAAACGAAGACATCCTGGAAGACA |
| | | TCGTGCTGACCCTGACGCTGTTCGAAGACCGCGAAATGATCGAAGAACGCCTG |
| | | AAGACGTATGCCCACCTGTTCGACGACAAAGTGATGAAGCAGCTGAAACGCCG |
| | | CCGCTACACAGGATGGGGACGCCTGAGCCGCAAACTGATCAACGGCATCCGCG |
| | | ACAAGCAGAGCGGAAAAACGATCCTGGACTTCCTGAAGTCGGACGGCTTCGCA |
| | | AACCGCAACTTCATGCAGCTGATCCATGACGACAGCCTGACATTCAAGGAAGA |
| | | CATCCAGAAAGCACAGGTGTCGGGACAGGGAGACTCCCTGCATGAACACATCG |
| | | CCAACCTGGCAGGCTCGCCGGCAATCAAAAAGGGAATCCTGCAGACCGTGAAA |
| | | GTGGTGGACGAACTGGTGAAGGTGATGGGCCGCCACAAACCGGAAAACATCGT |
| | | GATCGAAATGGCCCGCGAAAACCAGACCACGCAGAAGGGACAGAAAAACTCCC |
| | | GCGAACGCATGAAGCGCATCGAAGAAGGCATCAAAGAACTGGGAAGCCAGATC |
| | | CTGAAGGAACATCCGGTGGAAAACACGCAGCTGCAGAACGAAAAACTGTATCT |
| | | GTACTATCTGCAGAACGGCCGCGACATGTACGTGGACCAGGAACTGGACATCA |
| | | ACCGCCTGTCGGACTATGACGTGGACCACATCGTGCCGCAGTCCTTCCTGAAG |
| | | GACGACAGCATCGACAACAAAGTGCTGACACGCTCCGACAAGAACCGCGGAAA |
| | | ATCCGACAACGTGCCGAGCGAAGAAGTGGTGAAAAAGATGAAAAACTACTGGC |
| | | GCCAGCTGCTGAACGCCAAGCTGATCACCCAGCGCAAATTCGACAACCTGACG |
| | | AAGGCCGAACGCGGCGGACTGAGCGAACTGGACAAGGCAGGCTTCATCAAACG |
| | | CCAGCTGGTAGAAACCCGCCAGATCACGAAGCATGTGGCACAGATCCTGGACT |
| | | CGCGCATGAACACCAAATACGACGAAAACGACAAGCTGATCCGCGAAGTGAAA |
| | | GTGATCACGCTGAAATCGAAGCTGGTGTCCGACTTCCGCAAGGACTTCCAGTT |
| | | CTATAAAGTGCGCGAAATCAACAACTATCATCACGCCCACGACGCATACCTGA |
| | | ACGCCGTGGTGGGCACAGCACTGATCAAAAAGTACCCGAAGCTGGAAAGCGAA |
| | | TTTGTGTACGGAGACTATAAAGTGTACGACGTGCGCAAGATGATCGCCAAAAG |
| | | CGAACAGGAAATCGGAAAGGCCACCGCAAAGTATTTCTTCTACTCGAACATCA |
| | | TGAACTTCTTCAAGACAGAAATCACCCTGGCCAACGGCGAAATCCGCAAACGC |
| | | CCGCTGATCGAAACAAACGGCGAAACCGGAGAAATCGTGTGGGACAAGGGACG |
| | | CGACTTCGCAACGGTGCGCAAAGTGCTGTCCATGCCGCAGGTGAACATCGTGA |
| | | AAAAGACGGAAGTGCAGACAGGCGGATTCTCGAAAGAATCCATCCTGCCGAAG |
| | | CGCAACAGCGACAAACTGATCGCCCGCAAAAAGGACTGGGACCCGAAAAAGTA |
| | | TGGCGGATTCGACTCGCCGACCGTGGCCTACTCCGTGCTGGTAGTGGCAAAAG |
| | | TGGAAAAGGGCAAGTCGAAAAAGCTGAAGTCCGTGAAAGAACTGCTGGGAATC |
| | | ACGATCATGGAACGCTCCAGCTTCGAAAAGAACCCGATCGACTTCCTGGAAGC |
| | | AAAGGGCTATAAAGAAGTGAAAAAGGACCTGATCATCAAGCTGCCGAAATACA |
| | | GCCTGTTCGAACTGGAAAACGGACGCAAACGCATGCTGGCCTCGGCAGGCGAA |
| | | CTGCAGAAGGGAAACGAACTGGCCCTGCCGTCCAAATACGTGAACTTCCTGTA |
| | | TCTGGCATCCCATTACGAAAAACTGAAGGGAAGCCCGGAAGACAACGAACAGA |
| | | AGCAGCTGTTCGTGGAACAGCATAAACACTATCTGGACGAAATCATCGAACAG |
| | | ATCAGCGAATTTTCGAAGCGCGTGATCCTGGCCGACGCAAACCTGGACAAAGT |
| | | GCTGTCCGCCTACAACAAGCATCGCGACAAACCGATCCGCGAACAGGCAGAAA |
| | | ACATCATCCACCTGTTCACCCTGACGAACCTGGGAGCACCTGCAGCATTCAAA |
| | | TATTTCGACACAACCATCGACCGCAAGCGCTACACGAGCACAAAAGAAGTGCT |
| | | GGACGCAACCCTGATCCACCAGTCCATCACAGGACTGTACGAAACGAGAATCG |
| | | ACCTGAGCCAGCTGGGCGGAGACTAA |
| SEQ ID NO: 33 | oriT | CCGGCCAGCCTCGCAGAGCAGGATTCCCGTTGAGCACCGCCAGGTGCGAATAA |
| | | GGGACAGTGAAGAAGGAACACCCGCTCGCGGGTGGGCCTACTTCACCTATCCT |
| | | GCCC |
| SEQ ID NO: 34 | R6K origin | GATCTGAAGATCAGCAGTTCAACCTGTTGATAGTACGTACTAAGCTCTCATGT |
| | | TTCACGTACTAAGCTCTCATGTTTAACGTACTAAGCTCTCATGTTTAACGAAC |
| | | TAAACCCTCATGGCTAACGTACTAAGCTCTCATGGCTAACGTACTAAGCTCTC |
| | | ATGTTTCACGTACTAAGCTCTCATGTTTGAACAATAAAATTAATATAAATCAG |
| | | CAACTTAAATAGCCTCTAAGGTTTTAAGTTTTATAAGAAAAAAAAGAATATAT |
| | | AAGGCTTTTAAAGCTTTTAAGGTTTAACGGTTGTGGACAACAAGCCAGGGATG |
| | | TAACGCACTGAGAAGCCCTTAGAGCCTCTCAAAGCAATTTTGAGTGACACAGG |
| | | AACACTTAACGGCTGACA |
| SEQ ID NO: 35 | Upstream | TCCTGCTAATACTATCACAATCTCGCCCCGGGGCTCGGTAGCGGTAAAGTGTT |
| | homology #2 | CTATCAATTCCGCCAAACTTCCCCGAACGGTTTCTTCGTGGAGTTTGGAAATT |
| | | TCGCGAGACACGGTTGCCTGACGTTCAGTGCCGAAATATTCGGCAAACTGTGT |
| | | CAACGTTTTCAACAAGCGGTGAGGCGATTCATAAAACACCATAGTACGATGTT |
| | | CTTCTGCCAATGCCTTCAGTCGTGTCTGTCGTCCTTTCTTCTGAGGGAGGAAC |
| | | CCTTCGAAGCAGAACTTTTCATTCGGCAGTCCCGATGCTACCAATGCCGGAAC |
| | | AAAAGCTGTCGCTCCCGGCAAACATTGCACTTCGATACCGTTGCGCACACATT |
| | | CGCGGACTACCAAAAATCCCGGATCAGAAATCCCGGGTGTTCCGGCATCCGAA |
| | | ATCAATGCCACGGTTTCACCTGCCTTTATTCTATTAACAACACTTTCCACCGT |
| | | TTTATGTTCATTAAATTTGTGATGAGATTGCATTGCATTCTTTATTTCAAAGT |
| | | GTTTCAGCAAAATACCGGAAGTACGTGTATCCTCTGCCAAAATCAGATCGACC |
| | | TCTTTCAGCACCCTGATTGCCCGAAAGGTCATGTCCTCTAGATTTCCTACCGG |
| | | CGTAGGTACTACATATAACTTTCCCATAAACTTTTATGCTGATTGATTAAAAT |
| | | ACTTTTTTAGCAACTCCAAAAAGTCGATCATCGCCTCATTCTCTTCGTTTACG |
| | | CTGACTTTCGAGGCAAGAAATGCCACGGCAGTCTTATAAGAACTCATGACTGA |
| | | TATCTGCTCGATAACACGGTTCATCAGTTCGGGGTCTCCGCCGAATATCTCAC |
| | | GCGAGAAACGGAAAGAATCATTCAGGCTGATGGAATGACGTAATCCGGCAGCC |
| | | ATCTTTATGCTTTCGCCCAATACGGCAGTTTTCGGCTCCTCTATCAGAAGCGA |
| | | TTCGTCGTCTTCCGACTCATCTTCCGCTTCTTTCTCTTCCATTTCGTCTTCGA |
| | | CAACAGGTTCCTCAATTACTGCTTCTTCCACAACAGTCTGCGGTTCCTGTACT |
| | | ATCACAGGTTCATCTTCTCCCGGTGCTGTCGCTTCATTCTCTTCCACGACCTT |
| | | CTCTTCTATCACCGGACATTCAACTTCCTCTATTACAGGGGCTTGTTCTTCAA |
| | | CAATGGGGGCTTCACTTTCCGCTTCCGCTACAGGAGAAGGCGAGGCTTCCACC |
| | | GGCACAGCACTTATCTCTTCCGACAACTGTTCCAAACGCTCCTGCATACGTAG |
| | | GATGCTCCGCTTCAACAGTTCAGACAAAGTCTGAGTCGGCTCTTTAGAAAACG |
| | | TATTCATGAGTAGCTTCAGCTCATGAACATCCAGCTCAATATCTGTTAATAGC |
| | | TTCTGTTTCATTTCCATCCATTGTTATTATGGTGCGAATGTAGGAATAATTTA |
| | | TGCAATAACTTAAAATAATGGATAATTTAACAGTTCTTGTACATCGAATTGTT |
| | | TACCTTTGTCCACTCATTTATTTAATCAAGAAAGCACAAAAATCACATGGTA |
| SEQ ID NO: 36 | Downstream | AGGACGGACAGAAGATATAAACTCTATTTAAAACATATATGGAAAGAAAGAAG |
| | homology #2 | ATAACATTCGACAGTTTTATACGTGGTTCCATCGGATGTGTACTGGTGGTAGG |
| | | GATACTAATGCTTGTGGAACGGCTCAGCGGAGTGTTATTACCTTTCTTTATAG |
| | | CCTGGCTGATCGCCTACATGGTTTATCCGTTAGTCAAGTTTTTCCAGTATAAG |
| | | CTACGGTTAAAGAGCCGTATTGTTTCTATCTTCTGCTCCTTGTTTCTGATCAC |
| | | TCTTGTCGGAGTATCCTTATTTTATCTGTTGGTACCTCCCATGATTTCGGAAA |
| | | TAGGCAGGATGAATGACTTACTGGTAACCTACCTCACCAATGGAGCCGGGAAT |
| | | AATGTGCCCAAGAATCTTTCCGAATTCATTCATGAGAATATTGATCTTCAGGC |
| | | GCTCAACCGTATATTAAGCGAAGAGAATATTCTTGCAGCCATCAAAGATACAG |
| | | TGCCCAGAGTTTGGGCCCTGCTTGCGGAGTCGCTCAATATCCTGTTCAGCATT |
| | | CTCGCCTCCTTTATCATATTACTATATGTAATCTTTATATTGCTGGATTATGA |
| | | AGTCATAGCCGAAGGATGGCTGCATCTGCTGCCCAACAAGTATCGTACCTTCG |
| | | CATCCAACCTCGTACATGATGTACAGGACGGCATGAATCGGTATTTCCGTGGT |
| | | CAGGCACTGGTTGCCTTTTGTGTGGGGATTCTGTTCAGTATAGGCTTCCTTAT |
| | | TATCGATTTTCCGATGGCTATCGCTCTAGGGCTTTTCATTGGAGCGCTTAATA |
| | | TGGTTCCTTACCTGCAAATCATCGGTTTCCTCCCTACGGTCCTATTGGCGATC |
| | | CTTAAAGCTGCCGATACGGGAGAGAATTTCTGGATTATCATTGCATGCGCACT |
| | | GGCAGTCTTCGCCATCGTCCAAATTATACAGGATACTTTCCTCGTCCCCAAAA |
| | | TCATGGGAAAGATTACGGGGCTGAATCCTGCCATCATCCTTTTATCCCTTTCT |
| | | ATATGGGGTTCATTAATGGGAATGCTGGGCATGATCATTGCCTTGCCTCTGAC |
| | | TACTTTGATGCTTTCCTACTATCAACGCTTTATTATCAACAAAGAAAGAATAA |
| | | AATATGATGAAGTAGAAACTACTGATAATCAAGAAACAAGCGATAAAGAGGAA |
| | | AAATAATTGGAACTTTTTTCTACGAAACACTTGCAAATTAAATAAAAGTAACT |
| | | ACCTTTGCACCCGCAATCAGGAAATAACTGATTCGCAAATAAGGATTGATTCA |
| | | GTAGCTCAGCAGGTAGAGCACAACACTTTTAATGTTGGGGTCCTGGGTTCGAG |
| | | CCCCAGCTGGATCACTTAGAAGAAATGAAAATACTCCGACAAACTAAGACAAA |
| | | GCTCTTTAAACCAGTGGTTTAAGGAGCTTTTTTCTTACCCGCCTACGACAGAA |
| | | AAAAGACCTCAAAAAGCCACCCTGTGAAGATTAATCGTTACTAATTCGTTACG |
| | | GTTTTCCGTTACGATAAAAACCGTAACGAATTTATAGGTAAGTCGCTCATTTG |
| | | TCGAATATTGGCAGTC |
| SEQ ID NO: 37 | Bacteroides | CGAAATAAGCGTCAATATCCTCACTGCTTTTGAGGGTAACACACTTTCCGGAT |
| | origin of | TTGATTTCTTCCCTTGCCTTGTCAATCCTTGCTTGCAGCTCCGGGGTTATCAT |
| | replication | CAAATCTTCACGACCAACTTTTACCAAAGCGTAAATCTCGTTTTTCCTGCGAA |
| | | TTAGTACCTGCTCGCCATTGTCAGCTTTGGTGAAAGACGCTGCGAGGTTGTTA |
| | | CGGTATTCTCTTACTGATAATGCTTCCATATCTGTTTGCTTTTAAATTCAGCA |
| | | CAAAGATAGCTATATTTCAATAAAATACAAACATTTTGTACACAAACGTGTAC |
| | | ACGCCATAAAAACCCGTTTCCAATCCTACCGCCCGTTGGTTGGTTTTGCTTTG |
| | | CTCTTTTTCCCTATCGTTTTTCTTTTTCCGACAGTAAATCAACCACTTAGCTC |
| | | TATAAATTCCCCTTGTCTGTTATAAGCATCGTCTGTAATATGCTCTATCACTT |
| | | CTTCGCAAACGCCCATAAATAAAGGGATTAGAACATGTGTTTCCCCACACTTT |
| | | GGGAGTTTAGTCCCCACACTTTGGGAGTTTAAAAGTAAATTAGTCCCCACACT |
| | | TTGGGAGTTTTACCCCTCTTTTTTTGTTTTAATGTGGGGATTGTGTATATTTG |
| | | CAGCAAATCAATCGTTTATGAAAAAGAAACTACCTATTACCAAGAATAAAGAT |
| | | GTTGTTGTTTCATGGGTATATACATGGTCAAAACAGCAAGACATGTCCATACA |
| | | CGAACAAAGAATAGTTCTTCGCATACTGGAAGCGTGCCAAGCTGAACTAAAGG |
| | | GAGTAAAACTGAAAGATTATGCAGGCACGAAACGCAAGTTTGAGCATGGACTT |
| | | TGGGATGTTGATGCACAAATGCATGTATCTGACGTTATTTTTTCCGGACGTGA |
| | | TTACAATGAAATCATAGCCGCACTCGATTCTTTGGCAGGACGTTTTTTTACTT |
| | | ACGAAGATGACGAGGAATGGTGGAAGTGCGGATTTATATCTAACCCAAAATAT |
| | | AAAAAACGCACTGGCATTATAACCTTTCGTGTGTCTAACGACCTTTGGGATGT |
| | | GTTTACCAAGTTCGCCAAGGGGTACAGAGAATTTGAGTTAAACAAGGCTCTTG |
| | | CGCTACCTACTGGGTATTCCCTTAGGTTTTACATGTTAATGAGTGGGCAGGTG |
| | | TACCCTTTGGATATATCCCTTGAAAACTTGAAAGACCGTCTTGGCATACCTGC |
| | | GGACAAATACAAGGACAAGAACGGAAAAGACAGAATAGATCATTTTGAGGAAA |
| | | GAGTTTTGAAACCAGCAAAGGCTGCGCTTGATGAGAGCTGCCCATACACTTTC |
| | | AACTACGTGAAAGTGCGTGAAAACCCAAACAACAAACGTAGCAAGGTGACCGG |
| | | GTTTAGGTTCTACCCGGTCTATCAACCACAGTTCAGAGATGAAGAACTGGAAG |
| | | GGAAAGAATTGCAAGCAAAGGTTACAGCACGATATCAGATAGACAGCCATGTG |
| | | TACGAGTACCTCCGTTATTCCTGCGGCTTCACATCGGAAGAGATAAACCGGAA |
| | | CAAAGAGACATTCATAACGGCACAAGAAAAGATAACCGACCTTATCGGAGAAC |
| | | TGGCTCTCCTAAACGGAAAATCACGAGAAAAGAACAATCCGAAAGGGTGGATT |
| | | ATAAACGCCCTTAAAGGGAAAATCAAGGATAAGTAACAAGATTACCCGGCTGA |
| | | AACACTCCGGGTA |
| SEQ ID NO: 38 | tdk sgRNA | AAGATTTGTATCATTCGGATTTGGTAGACGATATTCTGAGTATTAAGACTTAT |
| | | TATGAACAACAGTGGCTCGATCGTGGTTTAAATATCAAATATATCAAGTTCCG |
| | | TCTTCCGCAAGAGGGAGTTTTGCAGGAACCGGATGTGGAAATTGAACTTGATC |
| | | CGTACCGTAGCTACAACCGTAGCAAGCGGAGCGGACTGCAAACGAGCAAATGA |
| | | TAAGTAATAAGTGACAAGTAATAAGTAACAAGTAATAATAGTAATTGGGTGGA |
| | | ACTATCAATTTTCCACTCTCAATTCTGAACTAATTATACTAGCCGATGAATCG |
| | | ATGTTTTAGAGCTAGAAATAGCAAGTTAAAATAAGGCTAGTCCGTTATCAACT |
| | | TGAAAAAGTGGCACCGAGTCGGTGCTTTTTT |
| SEQ ID NO: 39 | ErmG | ATGAACAAAGTAAATATAAAAGATAGTCAAAATTTTATTACTTCAAAATATCA |
| | resistance | CATAGAAAAAATAATGAATTGCATAAGTTTAGATGAAAAAGATAACATCTTTG |
| | gene | AAATAGGTGCAGGGAAAGGTCATTTTACTGCTGGATTGGTAAAGAGATGTAAT |
| | | TTTGTAACGGCGATAGAAATTGATTCTAAATTATGTGAGGTAACTCGTAATAA |
| | | GCTCTTAAATTATCCTAACTATCAAATAGTAAATGATGATATACTGAAATTTA |
| | | CATTTCCTAGCCACAATCCATATAAAATATTTGGCAGCATACCTTACAACATA |
| | | AGCACAAATATAATTCGAAAAATTGTTTTTGAAAGTTCAGCCACAATAAGTTA |
| | | TTTAATAGTGGAATATGGTTTTGCTAAAATGTTATTAGATACAAACAGATCAC |
| | | TAGCATTGCTGTTAATGGCAGAGGTAGATATTTCTATATTAGCAAAAATTCCT |
| | | AGGTATTATTTCCATCCAAAACCTAAAGTGGATAGCACATTAATTGTATTAAA |
| | | AAGAAAGCCAGCAAAAATGGCATTTAAAGAGAGAAAAAAATATGAAACTTTTG |
| | | TAATGAAATGGGTTAACAAAGAGTACGAAAAACTGTTTACAAAAAATCAATTT |
| | | AATAAAGCTTTAAAACATGCGAGAATATATGATATAAACAATATTAGTTTCGA |
| | | ACAATTTGTATCGCTATTTAATAGTTATAAAATATTTAACGGCTAA |
| SEQ ID NO: 40 | Carb | ATGAGTATTCAACATTTCCGTGTCGCCCTTATTCCCTTTTTTGCGGCATTTTG |
| | resistance | CCTTCCTGTTTTTGCTCACCCAGAAACGCTGGTGAAAGTAAAAGATGCTGAAG |
| | gene | ATCAGTTGGGTGCACGAGTGGGTTACATCGAACTGGATCTCAACAGCGGTAAG |
| | | ATCCTTGAGAGTTTTCGCCCCGAAGAACGTTTTCCAATGATGAGCACTTTTAA |
| | | AGTTCTGCTATGTGGCGCGGTATTATCCCGTATTGACGCCGGGCAAGAGCAAC |
| | | TCGGTCGCCGCATACACTATTCTCAGAATGACTTGGTTGAGTACTCACCAGTC |
| | | ACAGAAAAGCATCTTACGGATGGCATGACAGTAAGAGAATTATGCAGTGCTGC |
| | | CATAACCATGAGTGATAACACTGCGGCCAACTTACTTCTGACAACGATCGGAG |
| | | GACCGAAGGAGCTAACCGCTTTTTTGCACAACATGGGGGATCATGTAACTCGC |
| | | CTTGATCGTTGGGAACCGGAGCTGAATGAAGCCATACCAAACGACGAGCGTGA |
| | | CACCACGATGCCTGTAGCAATGGCAACAACGTTGCGCAAACTATTAACTGGCG |
| | | AACTACTTACTCTAGCTTCCCGGCAACAATTAATAGACTGGATGGAGGCGGAT |
| | | AAAGTTGCAGGACCACTTCTGCGCTCGGCCCTTCCGGCTGGCTGGTTTATTGC |
| | | TGATAAATCTGGAGCCGGTGAGCGTGGGTCTCGCGGTATCATTGCAGCACTGG |
| | | GGCCAGATGGTAAGCCCTCCCGTATCGTAGTTATCTACACGACGGGGAGTCAG |
| | | GCAACTATGGATGAACGAAATAGACAGATCGCTGAGATAGGTGCCTCACTGAT |
| | | TAAGCATTGGTAA |
| SEQ ID NO: 41 | AcrIIA2 | ATGACGCTGACCCGCGCTCAGAAAAAATACGCCGAGGCGATGCATGAGTTTAT |
| | | CAATATGGTTGATGACTTTGAAGAATCAACGCCTGACTTTGCAAAAGAGGTTC |
| | | TGCACGACTCCGACTATGTGGTCATTACAAAAAACGAGAAATATGCCGTGGCA |
| | | CTCTGTAGTCTCTCCACAGATGAATGTGAGTACGATACTAACTTGTATTTGGA |
| | | TGAAAAGCTCGTCGATTACAGCACAGTTGATGTCAACGGAGTGACATATTACA |
| | | TCAATATAGTGGAAACAAATGACATAGATGATCTTGAAATTGCGACCGACGAG |
| | | GACGAGATGAAGTCTGGAAACCAAGAGATTATTCTTAAGTCCGAACTGAAGTA |
| | | A |
| SEQ ID NO: 42 | Primer 5 | GAAGCCAACATGGCTGCGCACATAA |
| SEQ ID NO: 43 | Primer 6 | GATCACCGTCCACCGTGATACGTAC |
| SEQ ID NO: 44 | Cas9 | ATGGATAAGAAATACTCAATAGGCTTAGatATCGGCACAAATAGCGTCGGATG |
| | | GGCGGTGATCACTGATGAATATAAGGTTCCGTCTAAAAAGTTCAAGGTTCTGG |
| | | GAAATACAGACCGCCACAGTATCAAAAAAAATCTTATAGGGGCTCTTTTATTT |
| | | GACAGTGGAGAGACAGCGGAAGCGACTCGTCTCAAACGGACAGCTCGTAGAAG |
| | | GTATACACGTCGGAAGAATCGTATTTGTTATCTACAGGAGATTTTTTCAAATG |
| | | AGATGGCGAAAGTAGATGATAGTTTCTTTCATCGACTTGAAGAGTCTTTTTTG |
| | | GTGGAAGAAGACAAGAAGCATGAACGTCATCCTATTTTTGGAAATATAGTAGA |
| | | TGAAGTTGCTTATCATGAGAAATATCCAACTATCTATCATCTGCGAAAAAAAT |
| | | TGGTAGATTCTACTGATAAAGCGGATTTGCGCTTAATCTATTTGGCCTTAGCG |
| | | CATATGATTAAGTTTCGTGGTCATTTTTTGATTGAGGGAGATTTAAATCCTGA |
| | | TAATAGTGATGTGGACAAACTATTTATCCAGTTGGTACAAACCTACAATCAAT |
| | | TATTTGAAGAAAACCCTATTAACGCAAGTGGAGTAGATGCTAAAGCGATTCTT |
| | | TCTGCACGATTGAGTAAATCAAGACGATTAGAAAATCTCATTGCTCAGCTCCC |
| | | CGGTGAGAAGAAAAATGGCTTATTTGGGAATCTCATTGCTTTGTCATTGGGTT |
| | | TGACCCCTAATTTTAAATCAAATTTTGATTTGGCAGAAGATGCTAAATTACAG |
| | | CTTTCAAAAGATACTTACGATGATGATTTAGATAATTTATTGGCGCAAATTGG |
| | | AGATCAATATGCTGATTTGTTTTTGGCAGCTAAGAATTTATCAGATGCTATTT |
| | | TACTTTCAGATATCCTAAGAGTAAATACTGAAATAACTAAGGCTCCCCTATCA |
| | | GCTTCAATGATTAAACGCTACGATGAACATCATCAAGACTTGACTCTTTTAAA |
| | | AGCTTTAGTTCGACAACAACTTCCAGAAAAGTATAAAGAAATCTTTTTTGATC |
| | | AATCAAAAAACGGATATGCAGGTTATATTGATGGGGGAGCTAGCCAAGAAGAA |
| | | TTTTATAAATTTATCAAACCAATTTTAGAAAAAATGGATGGTACTGAGGAATT |
| | | ATTGGTGAAACTAAATCGTGAAGATTTGCTGCGCAAGCAACGGACCTTTGACA |
| | | ACGGCTCTATTCCCCATCAAATTCACTTGGGTGAGCTGCATGCTATTTTGAGA |
| | | AGACAAGAAGACTTTTATCCATTTTTAAAAGACAATCGTGAGAAGATTGAAAA |
| | | AATCTTGACTTTTCGAATTCCTTATTATGTTGGTCCATTGGCGCGTGGCAATA |
| | | GTCGTTTTGCATGGATGACTCGGAAGTCTGAAGAAACAATTACCCCATGGAAT |
| | | TTTGAAGAAGTTGTCGATAAAGGTGCTTCAGCTCAATCATTTATTGAACGCAT |
| | | GACAAACTTTGATAAAAATCTTCCAAATGAAAAAGTACTACCAAAACATAGTT |
| | | TGCTTTATGAGTATTTTACGGTTTATAACGAATTGACAAAGGTCAAATATGTT |
| | | ACTGAAGGAATGCGAAAACCAGCATTTCTTTCAGGTGAACAGAAGAAAGCCAT |
| | | TGTTGATTTACTCTTCAAAACAAATCGAAAAGTAACCGTTAAGCAATTAAAAG |
| | | AAGATTATTTCAAAAAAATAGAATGTTTTGATAGTGTTGAAATTTCAGGAGTT |
| | | GAAGATAGATTTAATGCTTCATTAGGTACCTACCATGATTTGCTAAAAATTAT |
| | | TAAAGATAAAGATTTTTTGGATAATGAAGAAAATGAAGATATCTTAGAGGATA |
| | | TTGTTTTAACATTGACCTTATTTGAAGATAGGGAGATGATTGAGGAAAGACTT |
| | | AAAACATATGCTCACCTCTTTGATGATAAGGTGATGAAACAGCTTAAACGTCG |
| | | CCGTTATACTGGTTGGGGACGTTTGTCTCGAAAATTGATTAATGGTATTAGGG |
| | | ATAAGCAATCTGGCAAAACAATATTAGATTTTTTGAAATCAGATGGTTTTGCC |
| | | AATCGCAATTTTATGCAGCTGATCCATGATGATAGTTTGACATTTAAAGAAGA |
| | | CATTCAAAAAGCACAAGTGTCTGGACAAGGCGATAGTTTACATGAACATATTG |
| | | CAAATTTAGCTGGTAGCCCTGCTATTAAAAAAGGTATTTTACAGACTGTAAAA |
| | | GTTGTTGATGAATTGGTCAAAGTAATGGGGCGGCATAAGCCAGAAAATATCGT |
| | | TATTGAAATGGCACGTGAAAATCAGACAACTCAAAAGGGCCAGAAAAATTCGC |
| | | GAGAGCGTATGAAACGAATCGAAGAAGGTATCAAAGAATTAGGAAGTCAGATT |
| | | CTTAAAGAGCATCCTGTTGAAAATACTCAATTGCAAAATGAAAAGCTCTATCT |
| | | CTATTATCTCCAAAATGGAAGAGACATGTATGTGGACCAAGAATTAGATATTA |
| | | ATCGTTTAAGTGATTATGATGTCGATCACATTGTTCCACAAAGTTTCCTTAAA |
| | | GACGATTCAATAGACAATAAGGTCTTAACGCGTTCTGATAAAAATCGTGGTAA |
| | | ATCGGATAACGTTCCAAGTGAAGAAGTAGTCAAAAAGATGAAAAACTATTGGA |
| | | GACAACTTCTAAACGCCAAGTTAATCACTCAACGTAAGTTTGATAATTTAACG |
| | | AAAGCTGAACGTGGAGGTTTGAGTGAACTTGATAAAGCTGGTTTTATCAAACG |
| | | CCAATTGGTTGAAACTCGCCAAATCACTAAGCATGTGGCACAAATTTTGGATA |
| | | GTCGCATGAATACTAAATACGATGAAAATGATAAACTTATTCGAGAGGTTAAA |
| | | GTGATTACCTTAAAATCTAAATTAGTTTCTGACTTCCGAAAAGATTTCCAATT |
| | | CTATAAAGTACGTGAGATTAACAATTACCATCATGCCCATGATGCGTATCTAA |
| | | ATGCCGTCGTTGGAACTGCTTTGATTAAGAAATATCCAAAACTTGAATCGGAG |
| | | TTTGTCTATGGTGATTATAAAGTTTATGATGTTCGTAAAATGATTGCTAAGTC |
| | | TGAGCAAGAAATAGGCAAAGCAACCGCAAAATATTTCTTTTACTCTAATATCA |
| | | TGAACTTCTTCAAAACAGAAATTACACTTGCAAATGGAGAGATTCGCAAACGC |
| | | CCTCTAATCGAAACTAATGGGGAAACTGGAGAAATTGTCTGGGATAAAGGGCG |
| | | AGATTTTGCCACAGTGCGCAAAGTATTGTCCATGCCCCAAGTCAATATTGTCA |
| | | AGAAAACAGAAGTACAGACAGGCGGATTCTCCAAGGAGTCAATTTTACCAAAA |
| | | AGAAATTCGGACAAGCTTATTGCTCGTAAAAAAGACTGGGATCCAAAAAAATA |
| | | TGGTGGTTTTGATAGTCCAACGGTAGCTTATTCAGTCCTAGTGGTTGCTAAGG |
| | | TGGAAAAAGGGAAATCGAAGAAGTTAAAATCCGTTAAAGAGTTACTAGGGATC |
| | | ACAATTATGGAAAGAAGTTCCTTTGAAAAAAATCCGATTGACTTTTTAGAAGC |
| | | TAAAGGATATAAGGAAGTTAAAAAAGACTTAATCATTAAACTACCTAAATATA |
| | | GTCTTTTTGAGTTAGAAAACGGTCGTAAACGGATGCTGGCTAGTGCCGGAGAA |
| | | TTACAAAAAGGAAATGAGCTGGCTCTGCCAAGCAAATATGTGAATTTTTTATA |
| | | TTTAGCTAGTCATTATGAAAAGTTGAAGGGTAGTCCAGAAGATAACGAACAAA |
| | | AACAATTGTTTGTGGAGCAGCATAAGCATTATTTAGATGAGATTATTGAGCAA |
| | | ATCAGTGAATTTTCTAAGCGTGTTATTTTAGCAGATGCCAATTTAGATAAAGT |
| | | TCTTAGTGCATATAACAAACATAGAGACAAACCAATACGTGAACAAGCAGAAA |
| | | ATATTATTCATTTATTTACGTTGACGAATCTTGGAGCTCCCGCTGCTTTTAAA |
| | | TATTTTGATACAACAATTGATCGTAAACGATATACGTCTACAAAAGAAGTTTT |
| | | AGATGCCACTCTTATCCATCAATCCATCACTGGTCTTTATGAAACACGCATTG |
| | | ATTTGAGTCAGCTAGGAGGTGACTGA |
| SEQ ID NO: 45 | LSEI_2368 | ATGCCAGATTTAGCACAGTCAACGTTTATTTTGCCGCAAGATGCGACACTGAC |
| | (example | CTCAGAACAGTCAGCATTGGAACAGCGAATTTGGGCCTTTATCAACGACAATC |
| | protein | ACAACACAACGTCAACACATCTATTGATCATTTCGGGTGATGCCGGTGCTGGT |
| | target) | AAAAGTGTGGTGTTGGATGCTGCTTTTGCGCAGTTGCAAAAAGCTGCCCGTGC |
| | | GACAAGCGGCGAGTTGGCTGGCACAGACAACAAGTTGTTGGTAAATCACAATG |
| | | AAATGCTGAAGATTTACAAAGAAATTGCTGGTACAAAGTCTTATTTTCGAAAG |
| | | AAAGACTTTATGAAACCAACACCATTTATTAACGCGTACCGCAAAGCCGGTAA |
| | | GCGGGCAGATGTCGTGCTGATCGATGAAGGCCATTTACTACTGACGATGCCTG |
| | | ATCCGTATAATAAATTCCGTGGGCATAATCAGCTTGCTGATATTCTTCAGTTG |
| | | GCTCGAGTGGTGGTGCTCGTGTTCGATTTCCACCAGCTAGTCAAGCTAAAGAG |
| | | TTTTTGGACACCTGCTTTATTGAAAAGAGTGACGCGCGATTACGCAGTGACAC |
| | | ATTATCATCTGACCGAACAGATGCGGGTAGGCGATGCGGCGGTGAATGATTGG |
| | | ATTGATCATTTTGTTCGTGGAAAAATGTTGCCTTTGCCACATCCGCAACATTT |
| | | TGATTTTCAAGTTTTTTCTGATGGTCAGCCGATGTATGACCTCATTCAGCGGC |
| | | GCGATGCCGAAACCGGCCTTTCGCGCATGGTCGCGACAGCTGACTATCCATTC |
| | | ACAGTGCTCGGTGGTAAAACCTGGTATGTCCAAGCTGGCTCTCTGCGGTTGCC |
| | | TTGGGACAAGATCAACTTCACCGATCGCCCGTGGGCACAACGTCCTGAGACAT |
| | | TGCATGAAGTTGGTTCCATTTATACTATTCAAGGCTTTGACTTGAATTACGCA |
| | | GGTGTGATTCTAGGGCCGAGTCTAGGCTATGATCCCTCAAAAGACCGACTTAC |
| | | GGTTGATCTGGCGCAGTACCAAGACAAGGAAGCATTCAAAAAGCGCCCGGATT |
| | | TGGCCGACACCACCGACGCTAAAGCCGCGATTATCATGAATGCCATTAATATT |
| | | CTGCTGAAGAGGGCGAAGCATGGGCTTTATCTTTACGCAGCTGATCCTGCTTT |
| | | GCGCCAACGGTTATTGCAATTGGCCAAATAA |
| SEQ ID NO: 46 | Upstream | CATACGAACACCTCCGTTAACAACAATACAGCATTCTGGCGTGCAAACGCTGC |
| | homology | TGTGACTTGATTTTGTCGGTACTTTTAAAACATTTTAAAAAAGAACATTAAAT |
| | arm 3.1 | ATACACAACAATTAACAGCATATTACATACTTTAACCCAGTAAACCGGTTACA |
| | | CTAGAGATAGTCAATTTCTCGAAAAAGTTATTCAAGAACTAGGAGTATTGAAA |
| | | TGCCTTCAATTGCTGATTACCCTCAACGCCATAGCTTGGCGTCATTTCAACAA |
| | | ACACCGCTCACTGAACTTGATGCGGGTTTATTTGCGCAACTCGGTTATTTGAA |
| | | CTTCAATTACCTGATCGGCCAGCCCTATGCGCGTTTTGCCGATTTAAATGACA |
| | | GTACCCGCCTAAACAGGGCTACCTTGACAACTTGGGCGATTCCAACTCATCAG |
| | | ATCATGTTAGACGCCATGCGTCACAGTGAACGTTTTGCCCGGGTTACTTGGGA |
| | | AAACTGGCTGGAAACTTGCAGCCATCGCAACGAAGAAGACTTCGCGGCCATCA |
| | | CATTCACATTAGCCCCCGGGGTTTATTGTGTCAGTTTTCGGGGGACAACCAAT |
| | | AAACTGGTTGGTTGGAAAGAAGATCTCAATATGAGCTTCATGCCAACGATTCC |
| | | AGCGCAGCGTCGGGAATTAAGTTACTTGATTAAACAAATCAGTCAGCATCCCG |
| | | GCACTTATTACCTGACTGGTCATTCCAAAGGCGGCAGTATCGCCACCTATGCG |
| | | TTTGACCACTTGCCACAACCATTAGCCAGTCAAGTTGCTCATGTCTATAGCTT |
| | | CGATGGGCCGAGCGGTGTCCCGCTTGATCCAAGCCATCGTGACCGTGTCACCA |
| | | AGCTCGTTCCACAAAGCTCCTTAATTGGCGTGAGTCTCGATCCAGCGATGAAT |
| | | TTTGAGGTTGTCAAAAGTCGCGTGAAGTTGTTCGGCCAACACGATGTTCTTAC |
| | | TTGGAACATTGCCGACACGACCTTCGCTCATTTGCCAACCACA |
| SEQ ID NO: 47 | Downstream | ACAACGGCGCTGCCGACCTGGGTCAGCATCAACATCGCCAACATGACGATATG |
| | homology | GATCAACTTCTTAGGCATCTGTGCGCCTCCTTCCATTGTTTCCGGCGTCTCTC |
| | arm 3.2 | TTCTTCCAACACCAGCTTAACAAGTTGACCCCGCGCAAAGGAATGATAATGTT |
| | | CGTTATTTAACAAGACATTTTTAACAGTGCATACTTATTGACATTATAGGTAT |
| | | TAATCTGTACATATAATGTAAAAAGCTCTATTTATAGGTAAATAAGGTCTTCA |
| | | AACCCAAAAGTAGCCTTGAAACAGCCGGCGTATTGCCAGTAAGTTTCAAGACT |
| | | ACTTATAGGTTATTTATTCAACTAATTAGTTTAAGCTTTGATGATTTTCCCAA |
| | | CTGCAATTGGTAAAGTAACGTGGGCATCGTAGTTCATGACAGTTCCTTGAACA |
| | | GTAGCCGGTAGCTTCAAGGTATCCGGCACACCATGAATATAAATGACCCTTTT |
| | | ATCGAGCTGCAAATCGTCACTGCCAAAAGCGGCTTTAAACGCTGTCTGCAATT |
| | | TCTTCAGATCAACGAGTTTGTCATAAGCATAGTGACCTTCTGCATCAGCCACT |
| | | GGGTAACTGTCATTTGGCACATGCATCTTGGCTGGTTCAGCGTCAAATGGCAC |
| | | CATCGTTGTACCGGAAACCGATTCTGTTTCTGGCAGATCAACGAAACCGTCAC |
| | | CATTAGCATCTTGTGCCGCTGTGGCGATTTCTGCAGGCTTGCCATCCGGGAAT |
| | | CCGTGGAAATGTTCCCAATGCTGCACATTAGCAGGTGTATCAAACATATCGAT |
| | | ATGAATCTTCATTTGCGCACCATCAATCGTGAACGTCGCGGCACCGTGCGCTG |
| | | CGCTGCCAATCTTCTCAGCGTTCAATGGCACAATTTCTGCTGTATACTTTTCA |
| | | GCCATGCAAACCACTCCTTTTAGATTTAGACTCAGTTTCAGCATAGTGCTAAC |
| | | TGGAACGACTCACAATCTATTGCATTTCAAAAGTGAGGTTGCAAGATTA |
| SEQ ID NO: 48 | sgRNA | CAGCATTGGAACAGCGAATTGTTTTAGAGCTAGAAATAGCAAGTTAAAATAAG |
| | | GCTAGTCCGTTATCAACTTGAAAAAGTGGCACCGAGTCGGTGCTTTTTT |
| SEQ ID NO: 49 | Rep origin #1 | CTATCGATAAGCTTAAGTGATTAGTCAAAGAATGGTGATGACAATTGTAAATT |
| | | CTATTTAATCACTTTGACTAGCAAATACTAACAACAAGACACACACACCAAAA |
| | | ATCAAAAATTCACTACTTTTAGTTAAAAACCACGTAACCACAAGAACTAATCC |
| | | AATCCATGTAATCGGGTTCTTCAAATATTTCTCCAAGATTTTCCTCCTCTAAT |
| | | ATGCTCAACTTAAATGACCTATTCAATAAATCTATTATGCTGCTAAATAGTTT |
| | | ATAGGACAAATAAGTATACTCTAATGACCTATAAAAGATAGAAAATTAAAAAA |
| | | TCAAGTGTTCGCTTCGCTCTCACTGCCCCTCGACGTTTTAGTAGCCTTTCCCT |
| | | CACTTCGTTCAGTCCAAGCCAACTAAAAGTTTTCGGGCTACTCTCTCCTTCTC |
| | | CCCCTAATAATTAATTAAAATCTTACTCTGTATATTTCTGCTAATCATTCGCT |
| | | AAACAGCAAAGAAAAAACAAACACGTATCATAGATATAAATGTAATGGCATAG |
| | | TGCGGGTTTTATTTTCAGCCTGTATCATAGCTAAACAAATCGAGTTGTGTGTC |
| | | CGTTTTAGGGCGTTCTGCTAGCTTGTTTAAAGTCTCTTGAATGAATGTATGCT |
| | | CTAAGTCAAAAGAATTTGTCAGCGCCTTTATATAGCTTTCTTTTTCTTCTTTT |
| | | TTTACTTTAATGATCGATAGCAACAATGATTTAACACTAGCAAGTTGAATGCC |
| | | ACCATTTCTTCCTGGTTTAATCTTAAAGAAAATTTCCTGATTCGCCTTCAGTA |
| | | CCTTCAGCAATTTATCTAATGTCCGTTCAGGAATGCCTAGCACTTCTCTAATC |
| | | TCTTTTTTGGTCGTCACTAAATAAGGCTTGTATACATCGCTTTTTTCGCTAAT |
| | | ATAAGCCATTAAATCTTCTTTCCATTCTGACAAATGAACACGTTGACGTTCGC |
| | | TTCTTTTTTTCTTGAATTTAAACCACCCTTGACGGACAAATAAATCTTTACTG |
| | | GTTAAATCACTTGATACCCAAGCTTTGCAAAGAATGGTAATGTATTCCCTATT |
| | | AGCCCCTTGATAGTTTTCTGAATAGGCACTTCTAACAATTTTGATTACTTCTT |
| | | TTTCTTCTAAGGGTTGATCTAATCGATTATTAAACTCAAACATATTATATTCG |
| | | CACGTTTCGATTGAATAGCCTGAACTAAAGTAGGCTAAAGAGAGGGTAAACAT |
| | | GACGTTATTACGCCCTATTAAACCCTTTTCTCCTGAAAATTTCGTTTCGTGCA |
| | | ATAAGAGATTAAACCAGGGTTCATCTACTTGTTTTTTGCCTTCTGTACCGCTT |
| | | AAAACCGTTAGACTTGAACGAGTAAAGCCCTTATTATCTGTTTGTTTGAAAGA |
| | | CCAATCTTGCCATTCTTTGAAAGAATAACGGTAATTAGGATCAAAAAATTCTA |
| | | CATTGTCCGTTCTTGGTATGCGAGCAATACCAAAATGATTACACGTTAGATCA |
| | | ACTGGCAAAGACTTTCCAAAATATTCTCGGATATTTTGCGAAATTATTTTGGC |
| | | TGCTTTGACAGATTTAAATTCTGATTTTGAAGTCACATAGACTGGCGTTTCTA |
| | | AAACAAAATATGCTTGATAACCTTTATCAGATTTGATAATCATAGTAGGCATA |
| | | AAACCTAAATCAATAGCGGTTGTTAAAATATCGCTTGCTGAAATAGTTTCTTT |
| | | TGCCGTGTGAATATCAAAATCAATAAAGAAGGTATTGATTTGTCTTAAATTGT |
| | | TTTCAGAATGTCCTTTCGTGTATGAACGGTTTTCGTCTGCATACGTTCCATAA |
| | | CGATAAACGTTGGGTGTCCAATGTGTAAATGTATCTTGATTTTCTTGAATCGC |
| | | TTCCTCGGAAGTCAGAACAACACCACGACCGCCAATCATGCTTGATTTTGAGC |
| | | GATACGCAAAAATAGCCCCTTTGCTTTTACCTGGCTTGGTAGTGATTGAGCGA |
| | | ATTTTACTATTTTTAAATTTGTACTTTAACAAGCCGTCATGAAGCACAGTTTC |
| | | TACAACAAAAGGGATATTCATTCAGCTGTTCTCCTTTCCTATAAATCCTATAA |
| | | AATAGGTTGTTTAATTAACTTGGTTTGCTTTTTCATTCAACTGTTTCAATATT |
| | | GCATGTTTTGAAAAAGATTTTTTTCCTTTATAAGTCAATTTTTTTCCACTAAT |
| | | CGAATAAATTATTTTGTTATTTTCTATTAACTTATATATATAATCTTCCCCCT |
| | | CCGAAGAAAAATACTTATCTGATTTTGTTTCTAAGTAGATATTTCTCTTTTCT |
| | | AACTCTTTCTTAAACGTTTCTAGTGTATAGATATTTGCTAATTTTCTTATCTC |
| | | CAATAAACTATTTTTTATATAAGTTTTACATTCATCATGATTCATACAAACTC |
| | | CACCTTCTATAAATGAATACAAAAAAAGCAATCAAACGATTTCCGATTGATTG |
| | | CTTAACAATTCTTAAATTCAGTAGCTTAGATACTTGAAAACTCTCTGATTTCC |
| | | CTATATAATGATAGTACGGTTATATACCGTCTTCAAACAAAGTTAATTAAATA |
| | | ACTTCTTACGAGGGAAGAGTTCATCTGACTAACTGATAAGCGTTGGTTTGGCA |
| | | ATCTTATCGGGCTATGCATTTATAAAATGTCGTCAAACATTTTATAAATGTGT |
| | | CATGGCTCTTTTTTCGTTTCTATTCAGTTCGTTGTTTCGTTATATCTAGTATA |
| | | CCGCTTTTAAAAAAAATAAGCAACGATTTCGTGCATTATTCACACGAAGTCAT |
| | | TGCTTTTTTCTTCTTCCATTTCTAAATCCAATGTTACTTGTTCTGATTCTGTT |
| | | TCTGGTTCTGGTTCTGTTGGCTCATTTGGGATTAAATCCACTACTAGCGTTGA |
| | | GTTAGTTAACTTTGCAATTTGTTCTAGTGTTTTTATGGTTGGATCTGATTTTC |
| | | CTGATTCTATTCGTGAATAATTTGATCTACTCATTTCTAATTCTTGGGGTACC |
| | | GCCAGCATTTCGGAAAAAAACCACGCTAAGGATTTTTTCTATAAAAAGAGCCG |
| | | TTATATTAAGAATAAAACGGCTCTTTTATACGTAAAGGACGTAAATTCATTTG |
| | | CCCAGTGTCATGTAATCCTTCAAATTTGTATTCTCCAAGAAAATTGATATGTT |
| | | CCCATCCTAACGGCCACGCATATGGCATTAAATCTTCTCTAAATTCTCCTCTT |
| | | GCTTTTAATTCTTCTACGGCTTTTTCCATATATACAGTGTTCCACACACTTAT |
| | | AGCGTTAATAATTATGTTTAGTGCACTAGCTCTTTGTAACTGGTCTTGGAGAG |
| | | CACGTTCTCTAAATTCTCCACGTTGTCCAAAAAATATAATTCTAGCTAATGCA |
| | | TTGATTGCTTCTCCTTTATTTAAACCTTTTTGAACCCGTCTCCTTACGGCTTT |
| | | ATTAGATATGTAATCCAGCGTAAAGAGGGTTTTCTCGATTCGTCCCATTTCTC |
| | | CAAGTGCTGTTGCGAGTTTATTTTGTCTTGCATATGATCCGAGCTTCCCCATG |
| | | ATAA |
| SEQ ID NO: 50 | Rep origin #2 | TCAGATCCTTCCGTATTTAGCCAGTATGTTCTCTAGTGTGGTTCGTTGTTTTT |
| | | GCGTGAGCCATGAGAACGAACCATTGAGATCATACTTACTTTGCATGTCACTC |
| | | AAAAATTTTGCCTCAAAACTGGTGAGCTGAATTTTTGCAGTTAAAGCATCGTG |
| | | TAGTGTTTTTCTTAGTCCGTTATGTAGGTAGGAATCTGATGTAATGGTTGTTG |
| | | GTATTTTGTCACCATTCATTTTTATCTGGTTGTTCTCAAGTTCGGTTACGAGA |
| | | TCCATTTGTCTATCTAGTTCAACTTGGAAAATCAACGTATCAGTCGGGCGGCC |
| | | TCGCTTATCAACCACCAATTTCATATTGCTGTAAGTGTTTAAATCTTTACTTA |
| | | TTGGTTTCAAAACCCATTGGTTAAGCCTTTTAAACTCATGGTAGTTATTTTCA |
| | | AGCATTAACATGAACTTAAATTCATCAAGGCTAATCTCTATATTTGCCTTGTG |
| | | AGTTTTCTTTTGTGTTAGTTCTTTTAATAACCACTCATAAATCCTCATAGAGT |
| | | ATTTGTTTTCAAAAGACTTAACATGTTCCAGATTATATTTTATGAATTTTTTT |
| | | AACTGGAAAAGATAAGGCAATATCTCTTCACTAAAAACTAATTCTAATTTTTC |
| | | GCTTGAGAACTTGGCATAGTTTGTCCACTGGAAAATCTCAAAGCCTTTAACCA |
| | | AAGGATTCCTGATTTCCACAGTTCTCGTCATCAGCTCTCTGGTTGCTTTAGCT |
| | | AATACACCATAAGCATTTTCCCTACTGATGTTCATCATCTGAACGTATTGGTT |
| | | ATAAGTGAACGATACCGTCCGTTCTTTCCTTGTAGGGTTTTCAATCGTGGGGT |
| | | TGAGTAGTGCCACACAGCATAAAATTAGCTTGGTTTCATGCTCCGTTAAGTCA |
| | | TAGCGACTAATCGCTAGTTCATTTGCTTTGAAAACAACTAATTCAGACATACA |
| | | TCTCAATTGGTCTAGGTGATTTTAATCACTATACCAATTGAGATGGGCTAGTC |
| | | AATGATAATTACTAGTCCTTTTCCTTTGAGTTGTGGGTATCTGTAAATTCTGC |
| | | TAGACCTTTGCTGGAAAACTTGTAAATTCTGCTAGACCCTCTGTAAATTCCGC |
| | | TAGACCTTTGTGTGTTTTTTTTGTTTATATTCAAGTGGTTATAATTTATAGAA |
| | | TAAAGAAAGAATAAAAAAAGATAAAAAGAATAGATCCCAGCCCTGTGTATAAC |
| | | TCACTACTTTAGTCAGTTCCGCAGTATTACAAAAGGATGTCGCAAACGCTGTT |
| | | TGCTCCTCTACAAAACAGACCTTAAAACCCTAAAGGCTTAAGTAGCACCCTCG |
| | | CAAGCTCGGTTGCGGCCGCAATCGGGCAAATCGCTGAATATTCCTTTTGTCTC |
| | | CGACCATCAGGCACCTGAGTCGCTGTCTTTTTCGTGACATTCAGTTCGCTGCG |
| | | CTCACGGCTCTGGCAGTGAATGGGGGTAAATGGCACTACAGGCGCCTTTTATG |
| | | GATTCATGCAAGGAAACTACCCATAATACAAGAAAAGCCCGTCACGGGCTTCT |
| | | CAGGGCGTTTTATGGCGGGTCTGCTATGTGGTGCTATCTGACTTTTTGCTGTT |
| | | CAGCAGTTCCTGCCCTCTGATTTTCCAGTCTGACCACTTCGGATTATCCCGTG |
| | | ACAGGTCATTCAGACTGGCTAATGCACCCAGTAAGGCAGCGGTATCATCAAC |
| SEQ ID NO: 51 | acrIIA4 | ATGACGGTAACGAGTATGTAATTAGCGAATCTGAAAACGAGAGCATTGTTGAG |
| | | AAATTCATTTCAGCTTTCAAAAACGGTTGGAATCAAGAGTACGAGGACGAAGA |
| | | AGAGTTCTACAACGATATGCAAACCATCACTTTGAAAAGTGAATTGAACTAA |
| SEQ ID NO: 52 | AbR#1 | ATGAACAAAAATATAAAATATTCTCAAAACTTTTTAACGAGTGAAAAAGTACT |
| | | CAACCAAATAATAAAACAATTGAATTTAAAAGAAACCGATACCGTTTACGAAA |
| | | TTGGAACAGGTAAAGGGCATTTAACGACGAAACTGGCTAAAATAAGTAAACAG |
| | | GTAACGTCTATTGAATTAGACAGTCATCTATTCAACTTATCGTCAGAAAAATT |
| | | AAAACTGAATACTCGTGTCACTTTAATTCACCAAGATATTCTACAGTTTCAAT |
| | | TCCCTAACAAACAGAGGTATAAAATTGTTGGGAGTATTCCTTACCATTTAAGC |
| | | ACACAAATTATTAAAAAAGTGGTTTTTGAAAGCCATGCGTCTGACATCTATCT |
| | | GATTGTTGAAGAAGGATTCTACAAGCGTACCTTGGATATTCACCGAACACTAG |
| | | GGTTGCTCTTGCACACTCAAGTCTCGATTCAGCAATTGCTTAAGCTGCCAGCG |
| | | GAATGCTTTCATCCTAAACCAAAAGTAAACAGTGTCTTAATAAAACTTACCCG |
| | | CCATACCACAGATGTTCCAGATAAATATTGGAAGCTATATACGTACTTTGTTT |
| | | CAAAATGGGTCAATCGAGAATATCGTCAACTGTTTACTAAAAATCAGTTTCAT |
| | | CAAGCAATGAAACACGCCAAAGTAAACAATTTAAGTACCGTTACTTATGAGCA |
| | | AGTATTGTCTATTTTTAATAGTTATCTATTATTTAACGGGAGGAAATAA |
| SEQ ID NO: 53 | AbR#2 | ATGAGCCATATTCAACGGGAAACGTCTTGCTCGAGGCCGCGATTAAATTCCAA |
| | | CATGGATGCTGATTTATATGGGTATAAATGGGCTCGCGATAATGTCGGGCAAT |
| | | CAGGTGCGACAATCTATCGATTGTATGGGAAGCCCGATGCGCCAGAGTTGTTT |
| | | CTGAAACATGGCAAAGGTAGCGTTGCCAATGATGTTACAGATGAGATGGTCAG |
| | | ACTAAACTGGCTGACGGAATTTATGCCTCTTCCGACCATCAAGCATTTTATCC |
| | | GTACTCCTGATGATGCATGGTTACTCACCACTGCGATCCCCGGGAAAACAGCA |
| | | TTCCAGGTATTAGAAGAATATCCTGATTCAGGTGAAAATATTGTTGATGCGCT |
| | | GGCAGTGTTCCTGCGCCGGTTGCATTCGATTCCTGTTTGTAATTGTCCTTTTA |
| | | ACAGCGATCGCGTATTTCGTCTCGCTCAGGCGCAATCACGAATGAATAACGGT |
| | | TTGGTTGATGCGAGTGATTTTGATGACGAGCGTAATGGCTGGCCTGTTGAACA |
| | | AGTCTGGAAAGAAATGCATAAGCTTTTGCCATTCTCACCGGATTCAGTCGTCA |
| | | CTCATGGTGATTTCTCACTTGATAACCTTATTTTTGACGAGGGGAAATTAATA |
| | | GGTTGTATTGATGTTGGACGAGTCGGAATCGCAGACCGATACCAGGATCTTGC |
| | | CATCCTATGGAACTGCCTCGGTGAGTTTTCTCCTTCATTACAGAAACGGCTTT |
| | | TTCAAAAATATGGTATTGATAATCCTGATATGAATAAATTGCAGTTTCATTTG |
| | | ATGCTCGATGAGTTTTTCTAA |
| SEQ ID NO: 54 | Inducible | CTTCAATAGAGTTCTTAACGTTAATCCGAAAAAAACTAACGTTAATATTAAAA |
| | promoter | AATAAGATCCGCTTGTGAATTATGTATAATTTGATTAGACTAAAGAATAGGAG |
| | system #1 | AAAGTATGATGATATTTAAAAAACTTTCTCGTTAAGATAGGTTGTTGGTGAGC |
| | | ATGTTATATACGGATGTATCGGTTTCCTTAATGCAAAATTTTGTTGCTATCTT |
| | | AGCTCACCAACAACCTATCTTAATTTTTCTATTATATAGATATATTCAAAGAA |
| | | AGATAACATTTAAACGGATCATATTAGATATTTTAATAGCGATTATTTTTTCA |
| | | ATATTATATCTGTTTATTTCAGATGCGTCATTACTTGTAATGGTATTAATGCG |
| | | ATTAGGGTGGCATTTTCATCAACAAAAAGAAAATAAGATAAAAACGACTGATA |
| | | CAGCTAATTTAATTCTAATTATCGTGATCCAGTTATTGTTAGTTGCGGTTGGG |
| | | ACTATTATTAGTCAGTTTACCATATCGATTATCAAAAGTGATTTCAGCCAAAA |
| | | TATATTGAACAATAGTGCAACAGATATAACTTTATTAGGTATTTTCTTTGCTG |
| | | TTTTATTTGACGGCTTGTTCTTTATATTATTGAAGAATAAGCGGACTGAATTA |
| | | CAACATTTAAATCAAGAAATCATTGAATTTTCGTTAGAAAAACAATATTTTAT |
| | | ATTTATATTTATTTTATTTATAGTAATAGAAATTATTTTAGCAGTTGGGAATC |
| | | TTCAAGGAGTAACAGCCACGATATTATTAACCATTATCATTATTTTTTGTGTC |
| | | CTTATCGGGATGACTTTTTGGCAAGTGATGCTTTTTTTGAAGGCTTATTCGAT |
| | | TCGCCAAGAAGCCAATGACCAATTGGTCCGGAATCAACAACTTCAAGATTATC |
| | | TAGTCAATATCGAACAGCAGTACACCGAATTACGGCGATTTAAGCATGATTAT |
| | | CAAAACATCTTATTATCGTTGGAGAGTTTTGCCGAAAAGGGCGATCAGCAACA |
| | | GTTTAAGGCGTATTACCAAGAATTATTAGCACAACGGCCAATTCAAAGTGAAA |
| | | TCCAAGGGGCAGTCATTGCACAACTCGACTACTTGAAAAATGATCCTATTCGA |
| | | GGATTAGTCATTCAAAAGTTTTTGGCAGCCAAACAGGCTGGTGTTACTTTAAA |
| | | ATTCGAAATGACCGAACCAATCGAATTAGCAACCGCTAATCTATTAACGGTTA |
| | | TTCGGATTATCGGTATTTTATTAGACAATGCGATTGAACAAGCCGTTCAAGAA |
| | | ACCGATCAATTGGTGAGTTGTGCTTTCTTACAATCTGATGGTTTAATCGAAAT |
| | | TACGATTGAAAATACGGCCAGTCAAGTTAAGAATCTCCAAGCATTTTCAGAGT |
| | | TAGGCTATTCAACGAAAGGCGCTGGTCGGGGGACTGGTTTAGCTAATGTGCAG |
| | | GATTTGATTGCCAAACAAACCAATTTATTCTTAGAAACACAGATTGAAAATAG |
| | | AAAGTTACGACAGACATTGATGATTACGGAGGAAACTTAATTTGTATCCCGTT |
| | | TATTTATTAGAGGATGATTTACAGCAACAAGCGATTTATCAGCAAATTATCGC |
| | | GAATACGATTATGATTAACGAATTTGCAATGACTTTAACATGCGCTGCCAGTG |
| | | ATACTGAGACATTGTTGGCGGCAATTAAGGATCAGCAACGAGGTTTATTCTTT |
| | | TTGGATATGGAAATTGAGGATAACCGCCAAGCCGGTTTAGAAGTGGCAACTAA |
| | | GATTCGGCAGATGATGCCGTTTGCGCAAATTGTCTTCATTACAACCCACGAGG |
| | | AACTGACATTATTAACGTTAGAACGAAAAATAGCGCCTTTAGATTACATTCTC |
| | | AAGGACCAAACAATGGCTGAAATCAAAAGGCAATTGATTGATGATCTATTGTT |
| | | AGCTGAGAAGCAAAACGAGGCGGCAGCGTATCACCGAGAAAATTTATTTAGTT |
| | | ATAAAATAGGTCCTCGCTTTTTCTCATTACCATTAAAGGAAGTTGTTTATTTA |
| | | TATACTGAAAAAGAAAATCCGGGTCATATTAATTTGTTAGCCGTTACCAGAAA |
| | | GGTTACTTTTCCAGGAAATTTAAATGCGCTGGAAGCCCAATATCCAATGCTCT |
| | | TTCGGTGTGATAAAAGTTACTTAGTTAACCTATCTAATATTGCCAATTATGAC |
| | | AGTAAAACACGGAGTTTAAAATTTGTAGATGGCAGTGAGGCAAAAGTCTCGTT |
| | | CCGGAAATCACGGGAACTAGTGGCCAAATTAAAACAAATGATGTAG |
| SEQ ID NO: 55 | P_{orfX} | ACGCCAAATGATCCCAGTAAAAAGCCACCCGCATGGCGGGTGGCTTTTTATTA |
| | promoter | GCCCTAGAAGGGCTTCCCACACGCATTTCAGCGCCTTAGTGCCTTAGTTTGTG |
| | | AATCATAGGTGGTATAGTCCCGAAATACCCGTCTAAGGAATTGTCAGATAGGC |
| | | CTAATGACTGGCTTTTATAATATGAGATAATGCCGACTGTACTTTTTACAGTC |
| | | GGTTTTCTAATGTCACTAACCTGCCCCGTTAGTTGAAGAAGGTTTTTATATTA |
| | | CAGCTCCAGATCTACCGGTGGGCCCATATTAACGTTTAACCGATAAAGTTGAA |
| | | CGTTAATATTTTTTTTGCGCAGAAATGGTAAATTGAAGCATAATAGTCTTGTA |
| | | AGGTATTTAGCTGGCTGGCGTAAAGTATGCTTTATAAAATAATAT |
| SEQ ID NO: 56 | RBS | AGGAG |
| SEQ ID NO: 57 | Inducible | GAATTCCCCGGCTTTAGGTATAGTGTGTATCTCAATCCTTGGTATATTGAAAA |
| | promoter | GAAAGACTAAAAATTGATAGATTATATTTCTTCAGAATGAATGGTATAATGAA |
| | system #2 | GTAATGAGTACTAAACAATCGGAGGTAAAGTGGTGTATAAAATTTTAATAGTT |
| | | GATGATGATCAGGAAATTTTAAAATTAATGAAGACAGCATTAGAAATGAGAAA |
| | | CTATGAAGTTGCGACGCATCAAAACATTTCACTTCCCTTGGATATTACTGATT |
| | | TTCAGGGATTTGATTTGATTTTGTTAGATATCATGATGTCAAATATTGAAGGG |
| | | ACAGAAATTTGTAAAAGGATTCGCAGAGAAATATCAACTCCAATTATCTTTGT |
| | | TAGTGCGAAAGATACAGAAGAGGATATTATAAACGGCTTAGGTATTGGTGGGG |
| | | ATGACTATATTACTAAGCCTTTTAGCCTTAAACAGTTGGTTGCAAAAGTGGAA |
| | | GCAAATATAAAGCGAGAGGAACGCAATAAACATGCAGTTCATGTTTTTTCAGA |
| | | GATTCGTAGAGATTTAGGACCAATTACATTTTATTTAGAAGAAAGGCGAGTCT |
| | | GTGTCAATGGTCAAACAATTCCACTGACTTGTCGTGAATACGATATTCTTGAA |
| | | TTACTATCACAACGAACTTCTAAAGTTTATACGAGAGAGGATATTTATGATGA |
| | | CGTATATGATGAATATTCTAATGCACTTTTTCGGTCAATCTCGGAGTATATTT |
| | | ATCAGATTAGGAGTAAGTTTGCACCATACGATATTAATCCGATAAAAACGGTT |
| | | CGGGGACTTGGGTATCAGTGGCATGGGTAAAAAATATTCAATGCGTCGACGGA |
| | | TATGGCAAGCTGTCATTGAAATTATCATAGGTACTTGTCTACTTATCCTGTTG |
| | | TTACTGGGCTTGACTTTCTTTCTACGACAAATTGGACAAATCAGTGGTTCAGA |
| | | AACTATTCGTTTATCTTTAGATTCAGATAATTTAACTATTTCTGATATCGAAC |
| | | GTGATATGAAACACTACCCATATGATTATATTATTTTTGACAATGATACAAGT |
| | | AAAATTTTGGGAGGACATTATGTCAAGTCGGATGTACCTAGTTTTGTAGCTTC |
| | | AAAACAGTCTTCACATAATATTACAGAAGGAGAAATTACTTATACTTATTCAA |
| | | GCAATAAGCATTTTTCAGTTGTTTTAAGACAAAACAGTATGCCTGAATTTACA |
| | | AATCATACGCTTCGTTCAATTTCTTATAATCAATTTACTTACCTTTTCTTTTT |
| | | TCTTGGTGAAATAATACTCATTATTTTTTCTGTCTATCATCTCATTAGAGAAT |
| | | TTTCTAAGAATTTTCAAGCCGTTCAAAAGATTGCATTGAAGATGGGGGAAATA |
| | | ACTACTTTTCCTGAACAAGAGGAATCAAAAATTATTGAATTTGATCAGGTTCT |
| | | GAATAACTTATATTCGAAAAGTAAGGAGTTAGCTTTCCTTATTGAAGCGGAGC |
| | | GTCATGAAAAACATGATTTATCCTTCCAGGTTGCTGCACTTTCACATGATGTT |
| | | AAGACACCTTTAACAGTATTAAAAGGAAATATTGAACTGCTAGAGATGACTGA |
| | | AGTAAATGAACAACAAGCTGATTTTATTGAGTCAATGAAAAATAGTTTGACTG |
| | | TTTTTGACAAGTATTTTAACACAATGATTAGTTATACAAAACTTTTGAATGAT |
| | | GAAAATGATTACAAAGCGACAATCTCCCTGGAGGATTTTTTGATAGATTTATC |
| | | AGTTGAGTTGGAAGAGTTGTCAACAACTTATCAAGTGGATTATCAGCTAGTTA |
| | | AAAAAACAGATTTAACCACTTTTTACGGAAATACATTAGCTTTAAGTCGAGCA |
| | | CTTATCAATATCTTTGTTAATGCCTGTCAGTATGCTAAAGAGGGTGAAAAAAT |
| | | AGTCAGTTTGAGTATTTATGATGATGAAAAATATCTCTATTTTGAAATCTGGA |
| | | ATAATGGTCATCCTTTTTCTGAACAAGCAAAAAAAAATGCTGGAAAACTATTT |
| | | TTCACAGAAGATACTGGACGTAGTGGGAAACACTATGGGATTGGACTATCTTT |
| | | TGCTCAAGGTGTAGCTTTAAAACATCAAGGAAACTTAATTCTCAGTAATCCTC |
| | | AAAAAGGTGGGGCAGAAGTTATCCTAAAAATAAAAAAGTAA |
| SEQ ID NO: 58 | P_{nisA} promoter | CTCCTGTTTTACAACCGGGTGTACATAGCGAAATACTTGTAATGCGTGGTGAT |
| | | GCACCTGAATCTTTCTTCGAAACAGATACCAAATCCAAGCTAAAATCTTTTGT |
| | | ACTCATTTTGAGTGCCTCCTTATAATTTATTTTGTAGTTCCTTCGAACGAAAT |
| | | CATTGTATCTAACAAACTTCAGAATTTAATCAGAGCCGTTTATTATGCTCGCG |
| | | TTATCGACAATAATATTATTACCAATACTTTCTCAAGATAGAATTAAGACTGT |
| | | TTTAGATTTGTTAATGTTTCTATTGTCAGTATAGTTATAAGACT |
| SEQ ID NO: 59 | Recombinase | ATGACCATGCTTGATTACAACACAGCGGTTCTGAATGAATATCAACGGCGAGA |
| | | ACGACTTGAAGATAAAGCCATTGCCGATTGGGAGTCCTATCACGGTACCGTCT |
| | | TGCCCAAAGATATGGATATCGAACAAGCGGAGGAGTTCTTGGCCACCGCCGAT |
| | | GAATATGAAGTTGATACAAAGAAACCTTGGCTCTATCAAAGCTGTGCTGCATC |
| | | GCGTTATGAGGGCGCCTTTAACAAAGACAAAGCGAAGGAATACTTGAAAGATT |
| | | GGATCAACATTCACGGCCCTGAGCGATTCTTAAAAGACGCTGCTAGTTCTACG |
| | | TATCCGAAAACAGAACTGGTTGAGATTTTCTTCGGCGGTGACAGCTTAGACGT |
| | | TATTGATTTCATGAAGAATCAAGGATTTCAGGAATGGAAATAGGAGGAGTAGC |
| | | ATATGACGACACAATATGACCTAAAAAAAATGCCAGTTAAGAAACTGATTGAG |
| | | ACGCAGACGATTAAGAATAAGTTTGCAGCGCTTCTGGACAAACGGGCACCACA |
| | | GTTTCTTTCATCAATTGCCAGCGCGGTAAGCCTTAATCCAAGCTTAGCCAGAG |
| | | TTGATCAGTTAAGTGTTATCAACTCGGCCCTGGTAGCAGCAACGCTCGATCTT |
| | | CCGGTTAACCCGAGCTTGGGTTTTGTCTACATCGTTCCATACAAGAACCAGGC |
| | | GCAGCCACAGATTGGTTATAAAGGCTATATCCAATTAGCTCAACGATCAGGAC |
| | | GGTATCAGCGCCTGACTGCTTTACCAATTTATGAAGATGAGTTCAAGAGCTGG |
| | | AACCCACTAACGGAGGAACTTGAGTACACGCCGAACTTCCACGATCGCAAAGC |
| | | AAGCGAAAAACCGGTTGGCTATGCCGCATCGTTCAAACTGACTAACGGTTTTG |
| | | AAAAGATGGTCTATTGGACTTATCAGCAAGTCGATGATCATCGCAAGCGTTTC |
| | | AGCAAATCTGGTGGTAGCGCGGAGCCCAAGGGCGTTTGGAAAGACAACTACGA |
| | | GGCTATGGCCCTGAAGACGGTAATCAAATCGCTGCTGACTAAGTGGGGTCCAA |
| | | TGACAACCGACATGCAAAGTGCGGTCAGTGCCGATGAAAAACCAGTCGAAGCT |
| | | GATCCAGAACTGAAGGATGTTACCCCCGAAGATCCTAACTCGATCGAGGATGC |
| | | ACTTAACGCTCCCGCTGAACCCGTCACAAAATCGGAGGTGAAGCCAGATGCTC |
| | | TTAAGCCAGACATTACCCACGACCCAAATGCAGGAAAACAACCAGAAATCTTT |
| | | GACGGTCAACAAGGATAATTATTACTCGCTGGATACCAGTTTCAAATATCAGT |
| | | CTGCTACCTGGTTTAAGAAGTTTCTGACATGCGAAGCGGAAGCGATGGCCGAG |
| | | TTGCAAGGTAAATGGATACCAAGAGGTGATCCGACTGCCTTGCTGGTTGGAAA |
| | | CTATCTACACAGCTATTTTGAATCCAAGCAAGCTCATGAGTCTTTTATCAAAG |
| | | GACACCCAGAGATGTTCTCAACTCGTGGATCATCAAAAGGACAACTGAAAGCC |
| | | ACGTATAAACAAGCTGATGCGATGATTGCCACGCTTGAAGCTGATGAGAATGT |
| | | TCAACGACTTTATCAGGGCGAAAAAGAAGAGATCCTGACCGGTGATCTGTTTG |
| | | GGGTCGAGTGGATGGGCAAGCTGGACTGCTTCGACTCCACAAAGTCATTCTTT |
| | | TTGGATCTAAAGACCACACAGTCGCTTCACAAGAAGTATTGGAAACCAGGAGA |
| | | ACGTCAACCAACCAGTTTCGTTGATGCCTATAACTATCAGCTTCAGATGGCGG |
| | | TTTATCAGGAGCTGGTTTACCAAAATTACGGAACGCGACCACGAGCCTTCATC |
| | | ATTGCCGTGACTAAGGAAGACGTGCCCGACCATGCCGTCATCGAAGTACCACA |
| | | GTACCGTATGGACGAGGCACTGGAAGAGATCCATGACAGCACCGAACACGTTG |
| | | AGGCGGTTAAATCCGGTCAGGTGCGTCCTCATCGCTGTGAGGCCTGTGATTAC |
| | | TGCAAGGCAACTAAACGAGTCGCCACAATTATCAGCATGGATGAGCTAGTCGA |
| | | GTAG |

TABLE 3
Strain Table

| Strain | | Strain | | |
|---|---|---|---|---|
| No.: | Organism | Name | Genotype | Source |
| Strain | E. coli | MACH1 | F- φ80(lacZ)ΔM15 ΔlacX74 hsdR(rK- | Thermo |
| No. 1 | | | mK+) ΔrecA1398 endA1 tonA | Fisher |
| | | | | Scientific, |
| | | | | Waltham, |
| | | | | MA |
| Strain | E. coli | MG1655 | F- lambda- ilvG- rfb-50 rph-1 | ATCC, |
| No. 2 | | | | Old Town |
| | | | | Manassas, |
| | | | | VA |
| Strain | E. coli | — | F- lambda- ilvG- rfb-50 rph-1 ΔtonA | * |
| No. 3 | | | | |
| Strain | E. coli | NEB | F′ proA + B + lacIq Δ(lacZ)M15 | New |
| No. 4 | | Stable | zzf::Tn10 (TetR) Δ(ara-leu) 7697 | England |
| | | | araD139 fhuA ΔlacX74 galK16 | Biolabs, |
| | | | galE15 e14- Φ80dlacZΔM15 recA1 | Ipswich, |
| | | | relA1 endA1 nupG rpsL (StrR) rph | MA |
| | | | spoT1 Δ(mrr-hsdRMS-mcrBC) | |
| Strain | E. coli | — | F- lambda- ilvG- rfb-50 rph-1 | * |
| No. 5 | | | ΔtonA::sfGfp-KanR | |
| Strain | E. coli | OneShot | F- Δlac169 rpoS(Am) robA1 creC510 | Thermo |
| No. 6 | | Pir2 | hsdR514 endA recA1 | Fisher |
| | | | uidA(ΔMluI)::pir | Scientific, |
| | | | | Waltham, |
| | | | | MA |
| Strain | E. coli | MFDpir | F- lambda- ilvG- rfb-50 rph-1 RP4-2- | Ferrières, |
| No. 7 | | | Tc::Mu1::aac(3)IV-aphA-nic35- | et al., J. |
| | | | Mu2::zeo dapA::(erm-pir) recA | Bacteriol. |
| | | | | (2010) |
| | | | | 192: 6418- |
| | | | | 6427 |
| Strain | B. | VPI-5482 | From source | ATCC, |
| No. 8 | thetaiotaomicron | | | Old Town |
| | | | | Manassas, |
| | | | | VA |
| Strain | Lactobacillus | ATCC | From source | ATCC, |
| No. 9 | paracasei | 334 | | Old Town |
| | | | | Manassas, |
| | | | | VA |
| Strain | Lactoccoccus | MG1363 | From source | Intact |
| No. 10 | lactis | | | Genomics |
| | | | | St. Louis, |
| | | | | MO |

*Made in-house at Caribou Biosciences, Inc., Berkeley CA

Although preferred embodiments of the subject methods have been described in some detail, it is understood that obvious variations can be made without departing from the spirit and the scope of the methods as defined by the appended claims.

1. A plasmid comprising:
a sequence encoding a programmable CRISPR-associated (Cas) protein operably linked to an inducible promoter;
a guide polynucleotide capable of forming a complex with the Cas protein upon expression of the Cas protein, wherein the complex is capable of targeting a selected target site;
a first polynucleotide sequence homologous to a 3′ region adjacent to the selected target site;
a second polynucleotide sequence homologous to a 5′ region adjacent to the selected target site;
a sequence for a selectable marker; and
control elements that provide for expression of the plasmid sequences in a selected host cell.

2. The plasmid of claim 1, wherein the first polynucleotide sequence and second polynucleotide sequence are operably linked 5′ and 3′, respectively, to a donor polynucleotide.

3. The plasmid of claim 1, wherein the Cas protein comprises a catalytically active Cas endonuclease capable of producing a double-strand break at the selected target site.

4. The plasmid of claim 3, wherein the Cas endonuclease comprises a Cas9.

5. The plasmid of claim 1, wherein the programmable Cas protein comprises a nickase capable of producing a single-strand break at the selected target site.

6. The plasmid of claim 5, wherein the nickase comprises a Cas9 nickase (nCas9).

7. The plasmid of claim 1, wherein the programmable Cas protein comprises a catalytically inactive Cas protein (dCas) capable of binding to the selected target site but incapable of producing a double-strand or single-strand break at the selected target site.

8. The plasmid of claim 7, wherein the dCas comprises dCas9.

9. The plasmid of claim 1 further comprising a sequence encoding an anti-CRISPR molecule operably linked to a promoter, wherein the anti-CRISPR molecule is capable of inhibiting the function of the programmable Cas protein.

10. The plasmid of claim 9, wherein the anti-CRISPR molecule is selected from the group consisting of an AcrIIA1, an AcrIIA1-2, an AcrILAZ, an AcrIIA4, and an AcrIIA5.

11. The plasmid of claim 9, wherein a constitutive promoter is operably linked to the sequence encoding the anti-CRISPR molecule.

12. The plasmid of claim 1, wherein the inducible promoter operably linked to the sequence encoding the programmable Cas protein comprises an inducible tetracycline promoter.

13. The plasmid of claim 1, wherein the sequence for the selectable marker is capable of imparting antibiotic resistance to the host cell transformed with the plasmid.

14.-20. (canceled)

21. The plasmid of claim 1, wherein the control elements comprise two or more origins of replication.

22. A prokaryotic host cell transformed with the plasmid of claim 1.

23. The prokaryotic host cell of claim 22, wherein the prokaryotic cell comprises a Proteobacteria cell.

24. The prokaryotic host cell of claim 23, wherein the prokaryotic cell comprises an Escherichia coli cell.

25. The prokaryotic host cell of claim 22, wherein the prokaryotic cell comprises a Bacteroidetes cell.

26. The prokaryotic host cell of claim 25, wherein the prokaryotic cell comprises a Bacteroides spp.

27-30. (canceled)

31. A method for editing a prokaryotic genome comprising:
transforming a selected prokaryotic cell with the plasmid of claim 1; and
culturing the cell under conditions whereby the components of the plasmid are expressed such that homologous recombination at the selected target site occurs,
thereby editing the prokaryotic genome.

32.-39. (canceled)

40. The method of claim 31, wherein the prokaryotic cell is transformed by a method selected from the group consisting of electroporation, chemical transformation, and conjugation.
